SEQUENCE LISTING (196 sequences)

SEQ ID NO: 1 - protein, 24 aa, synthetic construct
AEEAGIGDTPSLEDEAAGHVTQAR

SEQ ID NO: 2 - protein, 39 aa, synthetic construct
QAAAQPHTEIPEGTTAEEAGIGDTPSLEDEAAGHVTQAR

SEQ ID NO: 3 - protein, 30 aa, synthetic construct
STPTAEAEEAGIGDTPSLEDEAAGHVTQAR

SEQ ID NO: 4 - protein, 12 aa, synthetic construct
VQIVYKPVDLSK

SEQ ID NO: 5 - protein, 22 aa, synthetic construct
EAGIGDTPSLEDEAAGHVTQAR

SEQ ID NO: 6 - protein, 14 aa, synthetic construct
QPSGTGPEDTEGGR

SEQ ID NO: 7 - protein, 14 aa, synthetic construct
DVDESSPQDSPPSK

SEQ ID NO: 8 - protein, 15 aa, synthetic construct
ASPAQDGRPPQTAAR

SEQ ID NO: 9 - protein, 23 aa, synthetic construct
EATSIPGFPAEGAIPLPVDFLSK

SEQ ID NO: 10 - protein, 17 aa, synthetic construct
VSTEIPASEPDGPSVGR

SEQ ID NO: 11 - protein, 20 aa, synthetic construct
GQDAPLEFTFHVEITPNVQK

SEQ ID NO: 12 - protein, 9 aa, synthetic construct
GPSLGEDTK

SEQ ID NO: 13 - protein, 10 aa, synthetic construct
EADLPEPSEK

SEQ ID NO: 14 - protein, 12 aa, synthetic construct
LQTAPVPMPDLK

SEQ ID NO: 15 - protein, 10 aa, synthetic construct
TDHGAEIVYK

SEQ ID NO: 16 - protein, 8 aa, synthetic construct
VVQEGFLR

SEQ ID NO: 17 - protein, 7 aa, synthetic construct
HAPELLK

SEQ ID NO: 18 - protein, 15 aa, synthetic construct
HQLLGDLHQEGPPLK

SEQ ID NO: 19 - protein, 11 aa, synthetic construct
EQAHSEEHLGR

SEQ ID NO: 20 - protein, 14 aa, synthetic construct
AAFPGAPGEGPEAR

SEQ ID NO: 21 - protein, 8 aa, synthetic construct
GAAPPGQK

SEQ ID NO: 22 - protein, 7 aa, synthetic construct
GQANATR

SEQ ID NO: 23 - protein, 7 aa, synthetic construct
HQPGGGK

SEQ ID NO: 24 - protein, 19 aa, synthetic construct
HVPGGGSVQIVYKPVDLSK

SEQ ID NO: 25 - protein, 11 aa, synthetic construct
DEAAGHVTQAR

SEQ ID NO: 26 - protein, 8 aa, synthetic construct
VQIVYKPV

SEQ ID NO: 27 - protein, 15 aa, synthetic construct
DEAAGHVTQEPESGK

SEQ ID NO: 28 - protein, 6 aa, synthetic construct
DTEGGR

SEQ ID NO: 29 - protein, 10 aa, synthetic construct
DLHQEGPPLK

SEQ ID NO: 30 - protein, 10 aa, synthetic construct
DGRPPQTAAR

SEQ ID NO: 31 - protein, 7 aa, synthetic construct
DGPSVGR

SEQ ID NO: 32 - protein, 18 aa, synthetic construct
DAPLEFTFHVEITPNVQK

SEQ ID NO: 33 - protein, 8 aa, synthetic construct
DLPEPSEK

SEQ ID NO: 34 - protein, 12 aa, synthetic construct
DNITHVPGGGNK

SEQ ID NO: 35 - protein, 9 aa, synthetic construct
DHGAEIVYK

SEQ ID NO: 36 - protein, 9 aa, synthetic construct
DEVSASLAK

SEQ ID NO: 37 - protein, 12 aa, synthetic construct
TVMENFVAFVDK

SEQ ID NO: 38 - protein, 11 aa, synthetic construct
LVAASQAALGL

SEQ ID NO: 39 - protein, 12 aa, synthetic construct
AVMDDFAAFVEK

SEQ ID NO: 40 - protein, 13 aa, synthetic construct
VNVDEVGGEALGR

SEQ ID NO: 41 - protein, 10 aa, synthetic construct
TPSLPTPPTR

SEQ ID NO: 42 - protein, 8 aa, synthetic construct
GAAPPGQK

SEQ ID NO: 43 - protein, 24 aa, synthetic construct
  SITE 24: note = 13C6-15N4-Arginine
AEEAGIGDTPSLEDEAAGHVTQAR

SEQ ID NO: 44 - protein, 39 aa, synthetic construct; identical to SEQ ID NO: 2
  SITE 39: note = 13C6-15N4-Arginine

SEQ ID NO: 45 - protein, 30 aa, synthetic construct; identical to SEQ ID NO: 3
  SITE 30: note = 13C6-15N4-Arginine

SEQ ID NO: 46 - protein, 12 aa, synthetic construct
  SITE 12: note = 13C6-15N2-Lysine
VQIVYKPVDLSK

SEQ ID NO: 47 - protein, 22 aa, synthetic construct
  SITE 22: note = 13C6-15N4-Arginine
EAGIGDTPSLEDEAAGHVTQAR

SEQ ID NO: 48 - protein, 11 aa, synthetic construct
  SITE 11: note = 13C6-15N4-Arginine
DEAAGHVTQAR

SEQ ID NO: 49 - protein, 8 aa, synthetic construct
  SITE 6: note = 13C6-15N2-Lysine
VQIVYKPV

SEQ ID NO: 50 - protein, 14 aa, synthetic construct
  SITE 14: note = 13C6-15N4-Arginine
QPSGTGPEDTEGGR

SEQ ID NO: 51 - protein, 14 aa, synthetic construct
  SITE 14: note = 13C6-15N2-Lysine
DVDESSPQDSPPSK

SEQ ID NO: 52 - protein, 15 aa, synthetic construct
  SITE 15: note = 13C6-15N4-Arginine
ASPAQDGRPPQTAAR

SEQ ID NO: 53 - protein, 23 aa, synthetic construct
  SITE 23: note = 13C6-15N2-Lysine
EATSIPGFPAEGAIPLPVDFLSK

SEQ ID NO: 54 - protein, 17 aa, synthetic construct
  SITE 17: note = 13C6-15N4-Arginine
VSTEIPASEPDGPSVGR

SEQ ID NO: 55 - protein, 20 aa, synthetic construct
  SITE 20: note = 13C6-15N2-Lysine
GQDAPLEFTFHVEITPNVQK

SEQ ID NO: 56 - protein, 9 aa, synthetic construct
  SITE 9: note = 13C6-15N2-Lysine
GPSLGEDTK

SEQ ID NO: 57 - protein, 10 aa, synthetic construct
  SITE 10: note = 13C6-15N2-Lysine
EADLPEPSEK

SEQ ID NO: 58 - protein, 8 aa, synthetic construct
  SITE 8: note = 13C6-15N4-Arginine
VVQEGFLR

SEQ ID NO: 59 - protein, 7 aa, synthetic construct
  SITE 7: note = 13C6-15N2-Lysine
HAPELLK

SEQ ID NO: 60 - protein, 15 aa, synthetic construct
  SITE 15: note = 13C6-15N2-Lysine
HQLLGDLHQEGPPLK

SEQ ID NO: 61 - protein, 11 aa, synthetic construct
  SITE 11: note = 13C6-15N4-Arginine
EQAHSEEHLGR

SEQ ID NO: 62 - protein, 14 aa, synthetic construct
  SITE 14: note = 13C6-15N4-Arginine
AAFPGAPGEGPEAR

SEQ ID NO: 63 - protein, 15 aa, synthetic construct
  SITE 15: note = 13C6-15N2-Lysine
DEAAGHVTQEPESGK

SEQ ID NO: 64 - protein, 6 aa, synthetic construct
  SITE 6: note = 13C6-15N4-Arginine
DTEGGR

SEQ ID NO: 65 - protein, 10 aa, synthetic construct
  SITE 10: note = 13C6-15N2-Lysine
DLHQEGPPLK

SEQ ID NO: 66 - protein, 10 aa, synthetic construct
  SITE 10: note = 13C6-15N4-Arginine
DGRPPQTAAR

SEQ ID NO: 67 - protein, 7 aa, synthetic construct
  SITE 7: note = 13C6-15N4-Arginine
DGPSVGR

SEQ ID NO: 68 - protein, 18 aa, synthetic construct
  SITE 18: note = 13C6-15N2-Lysine
DAPLEFTFHVEITPNVQK

SEQ ID NO: 69 - protein, 8 aa, synthetic construct
  SITE 8: note = 13C6-15N2-Lysine
DLPEPSEK

SEQ ID NO: 70 - protein, 12 aa, synthetic construct
  SITE 12: note = 13C6-15N2-Lysine
LQTAPVPMPDLK

SEQ ID NO: 71 - protein, 10 aa, synthetic construct
  SITE 10: note = 13C6-15N2-Lysine
TDHGAEIVYK

SEQ ID NO: 72 - protein, 8 aa, synthetic construct
  SITE 8: note = 13C6-15N2-Lysine
GAAPPGQK

SEQ ID NO: 73 - protein, 7 aa, synthetic construct
  SITE 7: note = 13C6-15N4-Arginine
GQANATR

SEQ ID NO: 74 - protein, 7 aa, synthetic construct
  SITE 7: note = 13C6-15N2-Lysine
HQPGGGK

SEQ ID NO: 75 - protein, 19 aa, synthetic construct
  SITE 19: note = 13C6-15N2-Lysine
HVPGGGSVQIVYKPVDLSK

SEQ ID NO: 76 - protein, 12 aa, synthetic construct
  SITE 12: note = 13C6-15N2-Lysine
DNITHVPGGGNK

SEQ ID NO: 77 - protein, 9 aa, synthetic construct
  SITE 9: note = 13C6-15N2-Lysine
DHGAEIVYK

SEQ ID NO: 78 - protein, 9 aa, synthetic construct
  SITE 9: note = 13C6-15N2-Lysine
DEVSASLAK

SEQ ID NO: 79 - protein, 29 aa, synthetic construct
  SITE 29: note = 13C6-15N4-Arginine
DAGLKAEEAGIGDTPSLEDEAAGHVTQAR

SEQ ID NO: 80 - protein, 34 aa, synthetic construct
  SITE 29: note = 13C6-15N4-Arginine
DAGLKAEEAGIGDTPSLEDEAAGHVTQARMVSKS

SEQ ID NO: 81 - protein, 29 aa, synthetic construct
  SITE 24: note = 13C6-15N4-Arginine
AEEAGIGDTPSLEDEAAGHVTQARMVSKS

SEQ ID NO: 82 - protein, 44 aa, synthetic construct
  SITE 44: note = 13C6-15N4-Arginine
GAPGKQAAAQPHTEIPEGTTAEEAGIGDTPSLEDEAAGHVTQAR

SEQ ID NO: 83 - protein, 49 aa, synthetic construct
  SITE 44: note = 13C6-15N4-Arginine
GAPGKQAAAQPHTEIPEGTTAEEAGIGDTPSLEDEAAGHVTQARMVSKS

SEQ ID NO: 84 - protein, 44 aa, synthetic construct
  SITE 39: note = 13C6-15N4-Arginine
QAAAQPHTEIPEGTTAEEAGIGDTPSLEDEAAGHVTQARMVSKS

SEQ ID NO: 85 - protein, 35 aa, synthetic construct
  SITE 35: note = 13C6-15N4-Arginine
TSDAKSTPTAEAEEAGIGDTPSLEDEAAGHVTQAR

SEQ ID NO: 86 - protein, 40 aa, synthetic construct
  SITE 35: note = 13C6-15N4-Arginine
TSDAKSTPTAEAEEAGIGDTPSLEDEAAGHVTQARMVSKS

SEQ ID NO: 87 - protein, 35 aa, synthetic construct
  SITE 30: note = 13C6-15N4-Arginine
STPTAEAEEAGIGDTPSLEDEAAGHVTQARMVSKS

SEQ ID NO: 88 - protein, 17 aa, synthetic construct
  SITE 17: note = 13C6-15N2-Lysine
PGGGKVQIVYKPVDLSK

SEQ ID NO: 89 - protein, 22 aa, synthetic construct
  SITE 17: note = 13C6-15N2-Lysine
PGGGKVQIVYKPVDLSKVTSKC

SEQ ID NO: 90 - protein, 17 aa, synthetic construct
  SITE 12: note = 13C6-15N2-Lysine
VQIVYKPVDLSKVTSKC

SEQ ID NO: 91 - protein, 27 aa, synthetic construct
  SITE 27: note = 13C6-15N4-Arginine
QQRKREAGIGDTPSLEDEAAGHVTQAR

SEQ ID NO: 92 - protein, 32 aa, synthetic construct
  SITE 27: note = 13C6-15N4-Arginine
QQRKREAGIGDTPSLEDEAAGHVTQARMVSKS

SEQ ID NO: 93 - protein, 27 aa, synthetic construct
  SITE 22: note = 13C6-15N4-Arginine
EAGIGDTPSLEDEAAGHVTQARMVSKS

SEQ ID NO: 94 - protein, 16 aa, synthetic construct
  SITE 16: note = 13C6-15N4-Arginine
TPSLEDEAAGHVTQAR

SEQ ID NO: 95 - protein, 21 aa, synthetic construct
  SITE 16: note = 13C6-15N4-Arginine
TPSLEDEAAGHVTQARMVSKS

SEQ ID NO: 96 - protein, 16 aa, synthetic construct
  SITE 11: note = 13C6-15N4-Arginine
DEAAGHVTQARMVSKS

SEQ ID NO: 97 - protein, 13 aa, synthetic construct
  SITE 11: note = 13C6-15N4-Arginine
PGGGKVQIVYKPV

SEQ ID NO: 98 - protein, 18 aa, synthetic construct
  SITE 11: note = 13C6-15N2-Lysine
PGGGKVQIVYKPVDLSKV

SEQ ID NO: 99 - protein, 13 aa, synthetic construct
  SITE 6: note = 13C6-15N2-Lysine
VQIVYKPVDLSKV

SEQ ID NO: 100 - protein, 19 aa, synthetic construct
  SITE 19: note = 13C6-15N4-Arginine
REATRQPSGTGPEDTEGGR

SEQ ID NO: 101 - protein, 24 aa, synthetic construct
  SITE 19: note = 13C6-15N4-Arginine
REATRQPSGTGPEDTEGGRHAPEL

SEQ ID NO: 102 - protein, 19 aa, synthetic construct
  SITE 14: note = 13C6-15N4-Arginine
QPSGTGPEDTEGGRHAPEL

SEQ ID NO: 103 - protein, 19 aa, synthetic construct
  SITE 19: note = 13C6-15N2-Lysine
VDEDRDVDESSPQDSPPSK

SEQ ID NO: 104 - protein, 24 aa, synthetic construct
  SITE 19: note = 13C6-15N2-Lysine
VDEDRDVDESSPQDSPPSKASPAQ

SEQ ID NO: 105 - protein, 19 aa, synthetic construct
  SITE 14: note = 13C6-15N2-Lysine
DVDESSPQDSPPSKASPAQ

SEQ ID NO: 106 - protein, 20 aa, synthetic construct
  SITE 20: note = 13C6-15N4-Arginine
SPPSKASPAQDGRPPQTAAR

SEQ ID NO: 107 - protein, 25 aa, synthetic construct
  SITE 20: note = 13C6-15N4-Arginine
SPPSKASPAQDGRPPQTAAREATSI

SEQ ID NO: 108 - protein, 20 aa, synthetic construct
  SITE 15: note = 13C6-15N4-Arginine
ASPAQDGRPPQTAAREATSI

SEQ ID NO: 109 - protein, 28 aa, synthetic construct
  SITE 28: note = 13C6-15N2-Lysine
QTAAREATSIPGFPAEGAIPLPVDFLSK

SEQ ID NO: 110 - protein, 33 aa, synthetic construct
  MOD_RES 28: note = 13C6-15N2-Lysine
QTAAREATSIPGFPAEGAIPLPVDFLSKVSTEI

SEQ ID NO: 111 - protein, 28 aa, synthetic construct
  SITE 23: note = 13C6-15N2-Lysine
EATSIPGFPAEGAIPLPVDFLSKVSTEI

SEQ ID NO: 112 - protein, 22 aa, synthetic construct
  SITE 22: note = 13C6-15N4-Arginine
DFLSKVSTEIPASEPDGPSVGR

SEQ ID NO: 113 - protein, 27 aa, synthetic construct
  SITE 22: note = 13C6-15N4-Arginine
DFLSKVSTEIPASEPDGPSVGRAKGQD

SEQ ID NO: 114 - protein, 22 aa, synthetic construct
  SITE 17: note = 13C6-15N4-Arginine
VSTEIPASEPDGPSVGRAKGQD

SEQ ID NO: 115 - protein, 25 aa, synthetic construct
  SITE 25: note = 13C6-15N2-Lysine
VGRAKGQDAPLEFTFHVEITPNVQK

SEQ ID NO: 116 - protein, 30 aa, synthetic construct
  SITE 25: note = 13C6-15N2-Lysine
VGRAKGQDAPLEFTFHVEITPNVQKEQAHS

SEQ ID NO: 117 - protein, 25 aa, synthetic construct
  SITE 20: note = 13C6-15N2-Lysine
GQDAPLEFTFHVEITPNVQKEQAHS

SEQ ID NO: 118 - protein, 14 aa, synthetic construct
  SITE 14: note = 13C6-15N2-Lysine
GPEARGPSLGEDTK

SEQ ID NO: 119 - protein, 19 aa, synthetic construct
  SITE 14: note = 13C6-15N2-Lysine
GPEARGPSLGEDTKEADLP

SEQ ID NO: 120 - protein, 14 aa, synthetic construct
  SITE 9: note = 13C6-15N2-Lysine
GPSLGEDTKEADLP

SEQ ID NO: 121 - protein, 15 aa, synthetic construct
  SITE 15: note = 13C6-15N2-Lysine
GEDTKEADLPEPSEK

SEQ ID NO: 122 - protein, 20 aa, synthetic construct
  SITE 15: note = 13C6-15N2-Lysine
GEDTKEADLPEPSEKQPAAA

SEQ ID NO: 123 - protein, 15 aa, synthetic construct
  SITE 10: note = 13C6-15N2-Lysine
EADLPEPSEKQPAAA

SEQ ID NO: 124 - protein, 13 aa, synthetic construct
  SITE 13: note = 13C6-15N4-Arginine
PESGKVVQEGFLR

SEQ ID NO: 125 - protein, 18 aa, synthetic construct
  SITE 13: note = 13C6-15N4-Arginine
PESGKVVQEGFLREPGPP

SEQ ID NO: 126 - protein, 13 aa, synthetic construct
  SITE 8: note = 13C6-15N4-Arginine
VVQEGFLREPGPP

SEQ ID NO: 127 - protein, 12 aa, synthetic construct
  SITE 12: note = 13C6-15N2-Lysine
TEGGRHAPELLK

SEQ ID NO: 128 - protein, 17 aa, synthetic construct
  SITE 12: note = 13C6-15N2-Lysine
TEGGRHAPELLKHQLLG

SEQ ID NO: 129 - protein, 12 aa, synthetic construct
  SITE 7: note = 13C6-15N2-Lysine
HAPELLKHQLLG

SEQ ID NO: 130 - protein, 20 aa, synthetic construct
  SITE 20: note = 13C6-15N2-Lysine
PELLKHQLLGDLHQEGPPLK

SEQ ID NO: 131 - protein, 25 aa, synthetic construct
  SITE 20: note = 13C6-15N2-Lysine
PELLKHQLLGDLHQEGPPLKGAGGK

SEQ ID NO: 132 - protein, 20 aa, synthetic construct
  SITE 15: note = 13C6-15N2-Lysine
HQLLGDLHQEGPPLKGAGGK

SEQ ID NO: 133 - protein, 16 aa, synthetic construct
  SITE 16: note = 13C6-15N4-Arginine
PNVQKEQAHSEEHLGR

SEQ ID NO: 134 - protein, 21 aa, synthetic construct
  SITE 16: note = 13C6-15N4-Arginine
PNVQKEQAHSEEHLGRAAFPG

SEQ ID NO: 135 - protein, 16 aa, synthetic construct
  SITE 11: note = 13C6-15N4-Arginine
EQAHSEEHLGRAAFPG

SEQ ID NO: 136 - protein, 19 aa, synthetic construct
  SITE 19: note = 13C6-15N4-Arginine
EHLGRAAFPGAPGEGPEAR

SEQ ID NO: 137 - protein, 24 aa, synthetic construct
  SITE 19: note = 13C6-15N4-Arginine
EHLGRAAFPGAPGEGPEARGPSLG

SEQ ID NO: 138 - protein, 19 aa, synthetic construct
  SITE 14: note = 13C6-15N4-Arginine
AAFPGAPGEGPEARGPSLG

SEQ ID NO: 139 - protein, 20 aa, synthetic construct
  SITE 20: note = 13C6-15N2-Lysine
TPSLEDEAAGHVTQEPESGK

SEQ ID NO: 140 - protein, 25 aa, synthetic construct
  SITE 20: note = 13C6-15N2-Lysine
TPSLEDEAAGHVTQEPESGKVVQEG

SEQ ID NO: 141 - protein, 20 aa, synthetic construct
  SITE 15: note = 13C6-15N2-Lysine
DEAAGHVTQEPESGKVVQEG

SEQ ID NO: 142 - protein, 11 aa, synthetic construct
  SITE 11: note = 13C6-15N4-Arginine
GTGPEDTEGGR

SEQ ID NO: 143 - protein, 16 aa, synthetic construct
  SITE 11: note = 13C6-15N4-Arginine
GTGPEDTEGGRHAPEL

SEQ ID NO: 144 - protein, 11 aa, synthetic construct
  SITE 6: note = 13C6-15N4-Arginine
DTEGGRHAPEL

SEQ ID NO: 145 - protein, 15 aa, synthetic construct
  SITE 15: note = 13C6-15N2-Lysine
HQLLGDLHQEGPPLK

SEQ ID NO: 146 - protein, 20 aa, synthetic construct
  SITE 15: note = 13C6-15N2-Lysine
HQLLGDLHQEGPPLKGAGGK

SEQ ID NO: 147 - protein, 15 aa, synthetic construct
  SITE 10: note = 13C6-15N2-Lysine
DLHQEGPPLKGAGGK

SEQ ID NO: 148 - protein, 15 aa, synthetic construct
  SITE 15: note = 13C6-15N4-Arginine
ASPAQDGRPPQTAAR

SEQ ID NO: 149 - protein, 20 aa, synthetic construct
  SITE 15: note = 13C6-15N4-Arginine
ASPAQDGRPPQTAAREATSI

SEQ ID NO: 150 - protein, 15 aa, synthetic construct
  SITE 10: note = 13C6-15N4-Arginine
DGRPPQTAAREATSI

SEQ ID NO: 151 - protein, 12 aa, synthetic construct
  SITE 12: note = 13C6-15N4-Arginine
PASEPDGPSVGR

SEQ ID NO: 152 - protein, 17 aa, synthetic construct
  SITE 12: note = 13C6-15N4-Arginine
PASEPDGPSVGRAKGQD

SEQ ID NO: 153 - protein, 12 aa, synthetic construct
  SITE 7: note = 13C6-15N4-Arginine
DGPSVGRAKGQD

SEQ ID NO: 154 - protein, 23 aa, synthetic construct
  SITE 23: note = 13C6-15N2-Lysine
RAKGQDAPLEFTFHVEITPNVQK

SEQ ID NO: 155 - protein, 28 aa, synthetic construct
  SITE 23: note = 13C6-15N2-Lysine
RAKGQDAPLEFTFHVEITPNVQKEQAHS

SEQ ID NO: 156 - protein, 23 aa, synthetic construct
  SITE 18: note = 13C6-15N2-Lysine
DAPLEFTFHVEITPNVQKEQAHS

SEQ ID NO: 157 - protein, 13 aa, synthetic construct
  SITE 13: note = 13C6-15N2-Lysine
DTKEADLPEPSEK

SEQ ID NO: 158 - protein, 18 aa, synthetic construct
  SITE 13: note = 13C6-15N2-Lysine
DTKEADLPEPSEKQPAAA

SEQ ID NO: 159 - protein, 13 aa, synthetic construct
  SITE 8: note = 13C6-15N2-Lysine
DLPEPSEKQPAAA

SEQ ID NO: 160 - protein, 17 aa, synthetic construct
  SITE 17: note = 13C6-15N2-Lysine
SAKSRLQTAPVPMPDLK

SEQ ID NO: 161 - protein, 22 aa, synthetic construct
  SITE 17: note = 13C6-15N2-Lysine
SAKSRLQTAPVPMPDLKNVKSK

SEQ ID NO: 162 - protein, 17 aa, synthetic construct
  SITE 12: note = 13C6-15N2-Lysine
LQTAPVPMPDLKNVKSK

SEQ ID NO: 163 - protein, 15 aa, synthetic construct
  SITE 15: note = 13C6-15N2-Lysine
NAKAKTDHGAEIVYK

SEQ ID NO: 164 - protein, 20 aa, synthetic construct
  SITE 15: note = 13C6-15N2-Lysine
NAKAKTDHGAEIVYKSPVVS

SEQ ID NO: 165 - protein, 15 aa, synthetic construct
  SITE 10: note = 13C6-15N2-Lysine
TDHGAEIVYKSPVVS

SEQ ID NO: 166 - protein, 13 aa, synthetic construct
  SITE 13: note = 13C6-15N2-Lysine
IATPRGAAPPGQK

SEQ ID NO: 167 - protein, 18 aa, synthetic construct
  SITE 13: note = 13C6-15N2-Lysine
IATPRGAAPPGQKGQANA

SEQ ID NO: 168 - protein, 13 aa, synthetic construct
  SITE 8: note = 13C6-15N2-Lysine
GAAPPGQKGQANA

SEQ ID NO: 169 - protein, 12 aa, synthetic construct
  SITE 12: note = 13C6-15N4-Arginine
PPGQKGQANATR

SEQ ID NO: 170 - protein, 17 aa, synthetic construct
  SITE 12: note = 13C6-15N4-Arginine
PPGQKGQANATRIPAKT

SEQ ID NO: 171 - protein, 12 aa, synthetic construct
  SITE 7: note = 13C6-15N4-Arginine
GQANATRIPAKT

SEQ ID NO: 172 - protein, 12 aa, synthetic construct
  SITE 12: note = 13C6-15N2-Lysine
TENLKHQPGGGK

SEQ ID NO: 173 - protein, 17 aa, synthetic construct
  SITE 12: note = 13C6-15N2-Lysine
TENLKHQPGGGKVQIIN

SEQ ID NO: 174 - protein, 12 aa, synthetic construct
  SITE 7: note = 13C6-15N2-Lysine
HQPGGGKVQIIN

SEQ ID NO: 175 - protein, 24 aa, synthetic construct
  SITE 24: note = 13C6-15N2-Lysine
KDNIKHVPGGGSVQIVYKPVDLSK

SEQ ID NO: 176 - protein, 29 aa, synthetic construct
  SITE 24: note = 13C6-15N2-Lysine
KDNIKHVPGGGSVQIVYKPVDLSKVTSKC

SEQ ID NO: 177 - protein, 24 aa, synthetic construct
  SITE 19: note = 13C6-15N2-Lysine
HVPGGGSVQIVYKPVDLSKVTSKC

SEQ ID NO: 178 - protein, 17 aa, synthetic construct
  SITE 17: note = 13C6-15N2-Lysine
KIGSLDNITHVPGGGNK

SEQ ID NO: 179 - protein, 22 aa, synthetic construct
  SITE 17: note = 13C6-15N2-Lysine
KIGSLDNITHVPGGGNKKIETH

SEQ ID NO: 180 - protein, 17 aa, synthetic construct
  SITE 12: note = 13C6-15N2-Lysine
DNITHVPGGGNKKIETH

SEQ ID NO: 181 - protein, 14 aa, synthetic construct
  SITE 14: note = 13C6-15N2-Lysine
AKAKTDHGAEIVYK

SEQ ID NO: 182 - protein, 19 aa, synthetic construct
  SITE 14: note = 13C6-15N2-Lysine
AKAKTDHGAEIVYKSPVVS

SEQ ID NO: 183 - protein, 14 aa, synthetic construct
  SITE 9: note = 13C6-15N2-Lysine
DHGAEIVYKSPVVS

SEQ ID NO: 184 - protein, 14 aa, synthetic construct
  SITE 14: note = 13C6-15N2-Lysine
LATLADEVSASLAK

SEQ ID NO: 185 - protein, 17 aa, synthetic construct
  SITE 14: note = 13C6-15N2-Lysine
LATLADEVSASLAKQGL

SEQ ID NO: 186 - protein, 12 aa, synthetic construct
  SITE 9: note = 13C6-15N2-Lysine
DEVSASLAKQGL

SEQ ID NO: 187 - protein, 10 aa, synthetic construct
TPPSSGEPPK

SEQ ID NO: 188 - protein, 15 aa, synthetic construct
SGYSSPGSPGTPGSR

SEQ ID NO: 189 - protein, 23 aa, synthetic construct
ESPLQTPTEDGSEEPGSETSDAK

SEQ ID NO: 190 - protein, 15 aa, synthetic construct
  SITE 13: note = 13C6-15N2-Lysine
HVPGGGSVQIVYKPV

SEQ ID NO: 191 - protein, 21 aa, synthetic construct
  SITE 21: note = 13C6-15N2-Lysine
RVQSKIGSLDNITHVPGGGNK

SEQ ID NO: 192 - protein, 26 aa, synthetic construct
  SITE 21: note = 13C6-15N2-Lysine
RVQSKIGSLDNITHVPGGGNKGGSVQ

SEQ ID NO: 193 - protein, 21 aa, synthetic construct
  SITE 16: note = 13C6-15N2-Lysine
IGSLDNITHVPGGGNKGGSVQ

SEQ ID NO: 194 - protein, 20 aa, synthetic construct
  SITE 18: note = 13C6-15N2-Lysine
KDNIKHVPGGGSVQIVYKPV

SEQ ID NO: 195 - protein, 25 aa, synthetic construct
  SITE 18: note = 13C6-15N2-Lysine
KDNIKHVPGGGSVQIVYKPVDLSKV

SEQ ID NO: 196 - protein, 20 aa, synthetic construct
  SITE 13: note = 13C6-15N2-Lysine
HVPGGGSVQIVYKPVDLSKV

(19) United States
(12) Patent Application Publication
(10) Pub. No.: US 2026/0309720 A1
(43) Pub. Date: Oct. 8, 2026

(54) TAU ASSAY

(71) Applicant: EM SCIENTIFIC LIMITED, London (GB)

(72) Inventors: Andrew THOMPSON, London (GB); Ernestas SIRKA, London (GB); Jin XU, London (GB); Abhinav KUMAR, London (GB)

(21) Appl. No.: 19/483,452

(22) PCT Filed: May 10, 2024
(86) PCT No.: PCT/EP2024/062949

§ 371 Date: Nov. 11, 2025

(30) Foreign Application Priority Data
May 12, 2023 (GB) 2307071.7

(51) Int. Cl. G01N 33/68; C12Q 1/37; G01N 33/543

(52) U.S. Cl. CPC G01N 33/6851; C12Q 1/37; G01N 33/54326; G01N 33/54346; G01N 33/6896; G01N 2333/46; G01N 2333/96441; G01N 2333/96491; G01N 2333/976; G01N 2458/15; G01N 2800/2821

(57) ABSTRACT

This invention relates to the detection of Tau protein in body fluids using biomarker peptides and their detection by Multiplexed Liquid Chromatography Tandem Mass Spectrometry (LC-MS/MS) methods such as Selected Reaction Monitoring (SRM). In particular, the biomarker peptides are intended to distinguish between Tau protein produced by the Peripheral Nervous System and Tau protein produced by the Central Nervous System. This invention also relates to methods of using these peptide biomarkers for diagnosing and/or classifying the severity of a Neurodegenerative Disease, treatment of a Neurodegenerative Disease and assessing the efficacy of a treatment for a Neurodegenerative Disease, in particular for Tauopathies such as Alzheimer's disease. Also provided are a kit for diagnosing and/or classifying the severity of a Neurodegenerative disease, a kit for use in the treatment of a Neurodegenerative disease and a kit for assessing the efficacy of a treatment for a Neurodegenerative Disease in a subject.

### FIELD OF THE INVENTION

The invention is in the field of diagnosis and treatment of neurodegenerative disease, such as Alzheimer's disease.

### BACKGROUND

Neurodegenerative diseases, particularly Alzheimer's disease (AD), are characterized by Neurofibrillary Tangles of Tau protein and levels of these proteins are diagnostically significant. These proteins may also represent drug targets.

Neurofibrillary tangles, composed of intracellular aggregates of tau protein, are a key pathological feature of AD and other neurodegenerative diseases, collectively termed tauopathies. Tau is an intracellular microtubule-associated protein with several unique characteristics such as natively unfolded conformation, thermo-stability, acid-stability, and capability of post-translational modifications. Abnormally hyperphosphorylated tau is also a key feature of human tauopathies. In AD pathogenesis, accumulation of the amyloid-μ peptide (Aβ) interacts with the signalling pathways that regulate the phosphorylation of tau. Hyperphosphorylation of tau disrupts its normal function in regulating axonal transport and leads to the accumulation of neurofibrillary tangles and toxic species of soluble tau.

Tauopathies or tau-related pathologies include Pick disease, progressive supranuclear palsy, corticobasal degeneration, and argyrophilic grain disease. Emerging Tau-related disorders and pathologies include globular glial tauopathies, primary age-related tauopathy, which includes neurofibrillary tangle dementia, chronic traumatic encephalopathy (CTE), and aging-related tau astrogliopathy (Kovacs, Handb Clin Neurol, 145:355-368, “Tauopathies”, 2017). Currently there is no cure for AD and the few approved treatments have limited efficacy, serving at best to slow or delay progression but success of most current treatments is questionable.

Identification and development of new therapies for the treatment of AD and other tauopathies is also greatly affected by the lack of diagnostic, prognostic and predictive biomarkers that can be quickly and routinely tested in high throughput assays. Currently, AD can only be definitively diagnosed by brain biopsy or upon autopsy after a patient has died. Clearly, in clinical settings, brain biopsy is rarely performed and diagnosis is still primarily made based on the history of the symptoms and depends on a battery of neurological, psychometric and biochemical tests. These latter tests include assessment of ApoE e4 allele status and measurements of amyloid beta, tau and phospho-tau in cerebrospinal fluid.

More recently Positron Emission Tomography (PET) imaging using the non-specific tracer [¹⁸F]fluorodeoxyglucose (Tripathi & Murray, Neuroscientist 28(5):507-519. (doi: 10.1177/1073858421997035), “Alzheimer's Dementia: The Emerging Role of Positron Emission Tomography” 2022) can allow in vivo diagnosis in patients. Likewise Magnetic Resonance Imaging (MRI) can allow in vivo diagnosis in patients (Chandra et al., J Neurol 266(6):1293-1302, (doi: 10.1007/s00415-018-9016-3), “Magnetic resonance imaging in Alzheimer's disease and mild cognitive impairment”, 2019). However, both of these methods are slow and prohibitively expensive and are not really a suitable tool for assessing treatments or even as a routine tool for early screening of Neurodegenerative Disease.

Thus present methods are still unsatisfactory, not only for the early diagnosis of AD and other tauopathies, but also for predicting the progression of neurological disease which is important for recruitment of patients for clinical trials, or for designing new therapies and for predicting the effectiveness of current and new therapies.

The ideal diagnostic biomarkers should have high specificity for disease versus non-disease and high sensitivity to distinguish between disease types and stages. Prognostic biomarkers should reflect the intensity and severity of the pathological changes and predict their future course from a very early stage of the disease, before degeneration is observed, until advanced stages of the disease. Likewise, Pharmacodynamic biomarkers should give a reliable indication as to whether an administered therapy is efficacious based on the changes in the level of disease-related proteins in readily accessible body fluids such as blood, blood products including platelets, serum and most preferably plasma and CSF. It is also desirable that such pharmacodynamics biomarkers can provide guidance to clinicians when to stop treatment or switch to a different therapy.

A desirable assay needs to work with readily accessible body fluids, e.g. blood plasma in preference to CSF. A desirable assay needs to identify and quantify multiple possible biomarkers simultaneously in a single assay and a desirable assay needs to have sufficient throughput and be sufficiently cost-effective to allow it to be used for routine diagnosis at a population level and to be used for clinical studies where multiple assays need to be carried out for individual patients over the course of treatment.

Multiplexed Protein Target detection using Liquid Chromatography Tandem Mass Spectrometry (LC-MS/MS) methods such as Selected Reaction Monitoring (SRM) or Multiple Reaction Monitoring (MRM) have emerged as high-specificity, highly multiplexed alternative detection modalities to immunoassays for quantification of target proteins in biological samples. LC-MS/MS methods are highly desirable because LC-MS/MS methods provide both absolute structural specificity for the target protein and relative or absolute quantification of the target protein concentration when suitable heavy isotope internal standards are employed while also enabling large numbers of target biomarkers to be detected in a single assay. In contrast to immunoassays, LC-MS/MS does not generally require the manufacturing of biologics although immuno-precipitation of target proteins can increase sensitivity and does require appropriate ligands for target proteins. LC-MS/MS protein assays can be rapidly and inexpensively designed in silico based primarily on protein sequence information in contrast to the development of immunoassays, which require extensive screening and selection of antibodies or other protein affinity ligands. LC-MS/MS assays are highly multiplexed, with potential to run simultaneous assays for hundreds of proteins in a single sample analysis. In contrast to other proteomic technologies, LC-MS/MS allows complex assays to be developed for the identification of diagnostic proteins in complex diseases such as cancer, autoimmune, metabolic and neurodegenerative diseases. In particular, the development of a highly multiplexed LC-MS/MS assay that reproducibly identifies a specific set of proteins relevant to a clinical disease presents diagnostic advantages and efficiencies. In particular, traditional immunoassays can be challenging to develop for the discrimination of multiple different closely-related protein isoforms of a particular gene, whereas LC-MS/MS SRM methods, with appropriate design and selection of suitable peptides, can allow for discrimination and accurate quantification of multiple isoforms of the same protein within a single sample.

The Microtubule Associated Protein Tau (MAPT), which is often referred to as just the “Tau protein” (Canonical Isoform Uniprot ID: P10636) is a diagnostically significant protein that is expressed, as a result of alternative splicing from the MAPT gene, as multiple protein isoforms with different sizes ranging from 45 to 65 KDa. Isoforms show tissue-specific expression and it has been reported in the literature that High Molecular Weight Tau or so-called “Big Tau”, which broadly includes isoforms of Tau that comprise exon 4a of the gene and which produce a larger Tau protein, are expressed primarily in the Peripheral Nervous System (PNS) (Goedert, M. et al., Proc. Natl. Acad. Sci. U.S.A. 89, 1983-1987, “Cloning of a big tau microtubule associated protein characteristic of the peripheral nervous system.” 1992). Likewise, isoforms of Tau that do not comprise exon 4a are reported to be primarily expressed in the Central Nervous System (CNS). The role and function of Big Tau is not well understood but from a diagnostic perspective, the presence of Big Tau in plasma makes detection of increased levels of CNS Tau in plasma more challenging and requires discrimination between PNS Tau or Big Tau and CNS Tau or Low Molecular Weight Tau (LMW Tau).

U.S. Pat. No. 11,467,167 discloses an LC-SRM-MS blood plasma assay comprising 300 proteins reported to be associated with Alzheimer's disease. While this disclosure teaches assaying for these 300 proteins using a targeted LC-MS/MS method, this disclosure does not indicate how the data that is generated should be interpreted. There is no indication as to whether the targets proteins are up-regulated or down-regulated in Alzheimer's disease. In addition, this disclosure teaches that detection of Post-Translational Modifications (PTMs), such as phosphorylation of serine, threonine or tyrosine, are preferably avoided in the disclosed assay. The disclosed assay also preferably avoids detection of peptides that comprise cysteine. Detection of the Tau protein is disclosed in an SRM assay is disclosed in this patent application, however, the peptides selected (TPSLPTPPTR (SEQ ID NO: 41), TPPSSGEPPK (SEQ ID NO: 187), SGYSSPGSPGTPGSR (SEQ ID NO: 188), ESPLQTPTEDGSEEPGSETSDAK (SEQ ID NO: 189)) do not discriminate between PNS Tau/Big-Tau and other CNS isoforms of Tau that do not comprise exon 4a. No peptides that are present in exon 4a are mentioned in U.S. Pat. No. 11,467,167 and there is no mention of Tau isoforms or the need to discriminate between them in this disclosure. Likewise no peptides that span the splice junction between exon 4 and exon 5, i.e. peptides that would identify isoforms of Tau where exon 4a has been skipped are disclosed in U.S. Pat. No. 11,467,167.

U.S. Pat. No. 11,402,392 discloses an immunoprecipitation and LC-MS/MS SRM method to quantify phosphopeptides from Tau relative to Total Tau, specifically Tau phosphosites referred to as T205, T181, T217, T111, T153 and S208. U.S. Pat. No. 11,402,392 indicates that up-regulation of these phosphosites are indicative of disease and are indicative of need for therapeutic intervention. In this disclosure, native Tau peptides are quantified with reference to a spiked internal standard comprising ‘heavy’ recombinant 15N-labeled Tau protein. This patent discusses extensively the diagnostic use of phosphopeptides of Tau and does disclose peptides that can distinguish between some isoforms of CNS Tau. However, U.S. Pat. No. 11,402,392 does not mention High Molecular Weight or Big Tau and does not disclose any peptides derived from Exon 4a nor does U.S. Pat. No. 11,402,392 disclose any need to distinguish between PNS Tau and CNS Tau.

Similarly, a recent literature publication (Xu et al., J. Proteome Res. 20, 2299-2307, “Simultaneous Detection of Six Isoforms of Tau Protein in Human Cerebrospinal Fluid by Multidimensional Mass Spectrometry-Based Targeted Proteomics” 2021) discloses an SRM LC-MS/MS method to detect and quantify CNS isoforms of Tau and discriminate between them. This disclosure does not mention High Molecular Weight or Big Tau and does not disclose any peptides derived from Exon 4a nor does this publication disclose any need to distinguish between PNS Tau and CNS Tau.

A very recent literature publication (Gonzalez-Ortiz et al., Brain 00, 1-14, “Brain-derived tau: a novel blood-based biomarker for Alzheimer's disease-type neurodegeneration”, 2022) discusses the need to distinguish between PNS Tau (HMW Tau/Big Tau) and CNS Tau. This publication provides a method to discriminate between PNS Tau and CNS Tau based on antibodies that can bind selectively to the Exon 4/Exon 5 boundary sequence (which is not present in Big Tau). Using an antibody that binds to the exon boundary that is present specifically in the CNS Tau, the authors are able to discriminate between CNS Tau and PNS Tau when making measurements of total Tau in plasma or blood. One issue that is not completely clear from this publication is whether the antibodies generated are completely selective for the target CNS Tau protein. This is a general issue with antibody-based methods for detection of protein isoforms, which can often show cross-specificity for related proteins, i.e. antibodies are not often completely able to discriminate between isoforms. This publication does not suggest the use of LC-MS/MS SRM nor does it disclose peptide sequences specific for mass spectrometric identification of CNS Tau and discrimination of CNS Tau from PNS Tau using LC-MS/MS.

Co-pending patent application GB 2219461.7 by the present inventors discloses a novel multiplexed SRM assay for the detection of multiple proteins associated with Neurodegenerative disease. This panel also includes peptides for the detection of Total Tau and phosphopeptides of Tau as biomarkers of Neurodegenerative disease and, in particular, Alzheimer's Disease. This panel also does not disclose the use of peptides to distinguish between PNS isoforms of Tau and CNS isoforms of Tau.

Current plasma Total-Tau (T-Tau) assays, that do not distinguish between PNS Tau and CNS Tau splice variants, do not show a very strong correlation with Alzheimer's disease and as such have limited diagnostic utility (Clark C et al., Alzheimers Res Ther. 13:65. “Plasma neurofilament light and phosphorylated tau 181 as biomarkers of Alzheimer's disease pathology and clinical disease progression.” 2021; Barthélemy et al., J Exp Med. 217:e20200861, “Blood plasma phosphorylated-tau isoforms track CNS change in Alzheimer's disease.” 2020; Frank B et al., Alzheimers Dement. 18:1523-1536, “Plasma p-tau181 shows stronger network association to Alzheimer's disease dementia than neurofilament light and total tau.” 2022; Müller S et al., Sci Rep. 7:9529., “Tau plasma levels in subjective cognitive decline: Results from the DELCODE study.” 2017), while measurements of CSF Tau do reliably reflect neurodegeneration in Alzheimer's disease. Current plasma Total Tau concentrations show large overlaps between diagnostic groups (Mattsson N et al., Neurology. 87:1827-1835, “Plasma tau in Alzheimer disease.” 2016) and do not correlate with CSF Tau, suggesting that Total Tau in plasma and CSF Tau do not originate from the same tissue sources (Zetterberg H, Wilson D, Andreasson U, et al. Alzheimers Res Ther. 5:9, “Plasma tau levels in Alzheimer's disease.” 2013).

### SUMMARY

The present invention relates to protein biomarkers and methods of using these protein biomarkers for diagnosing and/or classifying the severity of a Neurodegenerative Disease, treatment of a Neurodegenerative Disease and assessing the efficacy of a treatment for a Neurodegenerative Disease, in particular for Alzheimer's disease. Also provided are a kit for diagnosing and/or classifying the severity of a Neurodegenerative disease, a kit for use in the treatment with a Neurodegenerative disease and a kit for assessing the efficacy of a treatment for a neurodegenerative disease in a subject.

### SUMMARY OF THE INVENTION

The present invention relates to methods for diagnosing and/or classifying the severity of a Neurodegenerative disease in a subject. It also relates to a method of treatment of a subject with a Neurodegenerative disease and a method for assessing the efficacy of a treatment for a neurodegenerative disease in a subject. Also provided are a kit for diagnosing and/or classifying the severity of a Neurodegenerative disease, a kit for use in the treatment with a Neurodegenerative disease and a kit for assessing the efficacy of a treatment for a neurodegenerative disease in a subject. The Neurodegenerative may be Alzheimer's disease. Methods and polypeptides are provided for quantifying the amount of one or more subtypes of Tau protein, e.g. Tau protein produced by the Peripheral Nervous System; Tau protein produced by the Central Nervous System; or substantially all subtypes of Tau Protein.

A key advantage of an LC-MS/MS method to discriminate between protein isoforms is that the discriminating peptide (as long as it is unique) can discriminate between protein isoforms with much higher specificity than antibody-based methods hence an LC-MS/MS SRM method for discrimination between Big Tau or PNS Tau and CNS Tau would be highly desirable.

Accordingly, the present disclosure provides LC-MS/MS SRM assays for reliable discrimination between CNS Tau and PNS Tau and hence reliable detection of Total CNS Tau in plasma enabling more reliable blood and plasma testing of CNS Tau.

For example, the present invention includes a specific clinical assay for the quantitative measurement of CNS Tau species and peptides derived from these protein variants in blood in a single sample and in a single assay using LC-MS/MS Multiple Reaction Monitoring that is scalable for routine use. The plasma biomarker test of the present invention may quantify peptides from different species of Microtubule-Associated Protein Tau (MAPT) where the species of Tau includes Total Tau protein, Central Nervous System Tau and Peripheral Nervous System Tau. The blood test can measure the absolute concentration of up to 100 or more peptides arising from the 3 classes of Tau protein present in blood, plasma or serum simultaneously generated by cleavage of these proteins with Trypsin or peptides arising from the 3 species of Tau present in blood, plasma or serum generated by cleavage of these proteins with a mixture of Trypsin and Asp-N. There are 117 different peptides of 5 amino acids or more among all the isoforms or subtypes of tau currently known. The specific combination of 3 protein classes and 36 peptide sequences is previously undisclosed to our knowledge. The multiplexed assay is definitive and can be accredited to existing regulatory standards to deploy in the clinic.

The present invention provides clinical utility as this assay may be used for development of drugs by enabling patient stratification in clinical trials, allowing the treatment efficacy of drugs in clinical development for neurodegeneration to be assessed and the assay may also be used to support neurodegenerative disease diagnosis and monitoring in the clinic as well as testing for disease recurrence.

The quantitative measurement of these low abundance protein species in a single sample specifically associated with Neurodegenerative Diseases such as Alzheimer's disease using mass spectrometry has not been previously disclosed or demonstrated. Simultaneous measurement of these proteins with sufficient sensitivity without interference from background matrix or the proteins themselves requires specific techniques to distinguish among the proteins. The assay was optimized for protein quantification and minimal interference among the proteins in the assay.

More specifically, in a first aspect of the invention, a method is provided for quantifying by mass spectrometry the amount of a subtype of the Microtubule Associate Protein Tau that is in a biological sample from a subject by measuring the absolute quantity of at least one target peptide that is substantially unique to the subtype being quantified with reference to at least one corresponding reference peptide(s), in a method comprising the steps:
(i) either, adding at least one reference peptide or at least one precursor reference peptide(s) or at least one reference polypeptide to the biological sample followed by incubating the biological sample with at least one protease to form a proteolytic digest of Tau proteins in the sample so that the protease releases the at least one target peptide from the Tau subtypes present and also releases the reference peptide from the precursor reference peptide or reference polypeptide; followed by isolating the Tau peptides and reference peptides from the proteolytic digest of the sample to form a target proteolytic digest;
   or, incubating the biological sample with at least one protease to form a proteolytic digest of Tau proteins in the sample so that the protease releases the at least one target peptide from the Tau subtypes present; followed by adding the at least one reference peptide(s) to the proteolytic digest and then isolating the Tau peptides from the proteolytic digest of the sample to form a target proteolytic digest;
(ii) assaying the target proteolytic digest for the presence of the at least one target proteolytic peptide that is substantially unique to the isoform of the Microtubule Associate Protein Tau and the at least one corresponding reference peptide(s), using mass spectrometry; optionally wherein the subtype is the Microtubule Associate Protein Tau expressed by the Central Nervous System or the Microtubule Associate Protein Tau expressed by the Peripheral nervous system.

Preferably, the subtype is the Microtubule Associate Protein Tau expressed by the Central Nervous System and the at least one target proteolytic peptide is one or more peptides selected from SEQ ID NO 1 to SEQ ID NO 5 or SEQ ID NO 25 or SEQ ID NO 26. The at least one target proteolytic peptide can be selected from the CNS tau peptides in table 1, table 2 and table 3. The reference peptides can comprise one or more peptides each comprising an amino acid sequence independently selected from SEQ ID NO 1 to SEQ ID NO 5 or SEQ ID NO 25 or SEQ ID NO 26, or a variant thereof. The reference peptides can comprise one or more peptides each comprising an amino acid sequence independently selected from the CNS tau peptides in table 1, table 2 and table 3, or a variant thereof. A suitable variant comprises at least one amino acid comprising heavy isotopes such as ²H, ¹³C ¹⁵N or ¹⁸O.

Alternatively, the subtype is the Microtubule Associate Protein Tau expressed by the Peripheral Nervous System and wherein the at least one target proteolytic peptide is one or more peptides selected from SEQ ID NO: 6 to SEQ ID NO 13 or SEQ ID NO 16 to SEQ ID NO 20 or SEQ ID NO 27 to SEQ ID NO 33. The at least one target proteolytic peptide can be selected from the PNS tau peptides in table 1, table 2 and table 3. The reference peptides can comprise one or more peptides each comprising an amino acid sequence independently selected from SEQ ID NO: 6 to SEQ ID NO 13 or SEQ ID NO 16 to SEQ ID NO 20 or SEQ ID NO 27 to SEQ ID NO 33, or a variant thereof. The reference peptides can comprise one or more peptides each comprising an amino acid sequence independently selected from the PNS tau peptides in table 1, table 2 and table 3 or a variant thereof. A suitable variant comprises at least one amino acid comprising heavy isotopes such as ²H, ¹³C ¹⁵N or ¹⁸O.

The method can also be used to quantify the amount of Microtubule Associate Protein Tau of substantially all subtypes (“Total Tau”) using target proteolytic peptides that are common to substantially all subtypes of Microtubule Associate Protein Tau. Suitable target peptides include one or more selected from SEQ ID NO:14; SEQ ID NO: 15; SEQ ID NO: 21 to SEQ ID NO: 24; SEQ ID NO: 34 to SEQ ID NO: 36. Suitable target peptides can further include SEQ ID NO: 188. The reference peptides can comprise one or more peptides each comprising an amino acid sequence independently selected from SEQ ID NO: 6 to SEQ ID NO 13 or SEQ ID NO 16 to SEQ ID NO 20 or SEQ ID NO 27 to SEQ ID NO 33, or a variant thereof. Suitable reference peptides can further include a peptide comprising the amino acid sequence SEQ ID NO: 188 or a variant thereof. A suitable variant comprises at least one amino acid comprising heavy isotopes such as ²H, ¹³C ¹⁵N or ¹⁸O.

Preferably, in the first aspect of invention, the reference peptides comprise one or more peptides each comprising an amino acid sequence independently selected from SEQ ID NO: 43 to SEQ ID NO: 186, such as SEQ ID NO: 43 to SEQ ID NO: 78.

Alternatively, in the first aspect of the invention, and more preferably, precursor reference peptides are used and the precursor reference peptides comprise one or more peptides each comprising an amino acid sequence independently selected from SEQ ID NO: 79 to SEQ ID NO: 186 from Table 8.

Further, in the first aspect of the invention, the reference polypeptides comprise one or more polypeptides selected from Table 7.

In the first aspect of the invention, the at least one protease is trypsin, optionally in combination with Lys-C and/or AspN.

In the first aspect of the invention when Trypsin is used in combination with AspN, the target proteolytic peptides comprise the sequence DEAAGHVTQAR (SEQ ID NO: 25) and/or the sequence VQIVYKPV (SEQ ID NO: 26) when the subtype is the Microtubule Associate Protein Tau expressed by the Central Nervous System.

Preferably, in the first aspect of the invention, at least one of the one or more reference peptides, precursor reference peptides or reference polypeptides comprise heavy isotopes and, more preferably, the heavy isotopes are selected from ²H, ¹³C ¹⁵N or ¹⁸O.

All the features of the first aspect of the present invention apply mutatis mutandis to the second aspect of the present invention and vice versa.

The method for quantifying by mass spectrometry the amount of a subtype of the Microtubule Associate Protein Tau in a sample can be used in methods for diagnosing and/or classifying the severity of neurodegenerative disease in a subject, described herein.

In a second aspect of the invention, a method is provided for diagnosing and/or classifying the severity of neurodegenerative disease in a subject, the method comprising:
i. Quantifying the absolute amount of the subtypes of the Microtubule Associate Protein Tau that are expressed by the Central Nervous System present in a biological sample from the subject by measuring the absolute quantity of at least one peptide that is substantially unique to the subtypes of the Microtubule Associate Protein Tau that are expressed by the Central Nervous System in a method comprising the steps:
   a. preparing the biological sample from the subject for assay by adding a predetermined amount of reference peptides or precursor reference peptides or reference polypeptides to the sample;
   b. isolating Tau proteins and the reference peptides or precursor reference peptides or reference polypeptides from the sample; then incubating the isolated target proteins and reference peptides or polypeptides with a protease to form a target proteolytic digest; or
   c. incubating the sample with a protease to form a proteolytic digest of the proteins and reference peptides or precursor reference peptides or reference polypeptides in the sample; and then isolating the target peptides and reference peptides from the proteolytic digest of the sample to form a target proteolytic digest;
ii. assaying the target proteolytic digest of step (i) for the presence of at least one target proteolytic peptide selected from a panel of peptides derived from the subtypes of the Microtubule Associate Protein Tau that are expressed by the Central Nervous System, wherein the presence of said at least one target proteolytic peptide(s) is assayed for using mass spectrometry with reference to a corresponding reference proteolytic peptide or polypeptide; and
iii. diagnosing and/or classifying the severity of the neurodegenerative disease of the subject according to the relative concentration of at least one target proteolytic peptide measured in the sample.

In one embodiment of the second aspect of the invention, the proteolytic digest is generated by contacting the sample with the protease Trypsin or Trypsin combined with LysC.

In embodiments of the second aspect of the invention where the protease is selected from Trypsin or Trypsin combined with LysC, preferably, the assaying step (ii) assays for the presence of at least one target proteolytic peptide selected from the group of peptides consisting of:

(SEQ ID NO: 1)
AEEAGIGDTPSLEDEAAGHVTQAR
(SEQ ID NO: 2)
QAAAQPHTEIPEGTTAEEAGIGDTPSLEDEAAGHVTQAR
(SEQ ID NO: 3)
STPTAEAEEAGIGDTPSLEDEAAGHVTQAR
(SEQ ID NO: 4)
VQIVYKPVDLSK
(SEQ ID NO: 5)
EAGIGDTPSLEDEAAGHVTQAR

In a second embodiment of the second aspect of the invention, the proteolytic digest is generated proteolytic digest is generated by contacting the sample with the protease Trypsin combined with AspN or by contacting the sample with Trypsin combined with LysC and AspN.

In embodiments of the second aspect of the invention where the protease is selected from Trypsin combined with AspN or Trypsin combined with LysC and AspN, preferably, the assaying step (ii) assays for the presence of at least one target proteolytic peptide selected from the group of peptides consisting of:

(SEQ ID NO: 25)
DEAAGHVTQAR
(SEQ ID NO: 26)
VQIVYKPV

In a third aspect of the invention, a method is provided for diagnosing and/or classifying the severity of neurodegenerative disease in a subject, the method comprising:
i. Quantifying the absolute amount of the subtypes of the Microtubule Associate Protein Tau that are expressed by the Central Nervous System present in a biological sample from the subject by measuring the absolute quantity of at least one peptide that is substantially unique to the subtypes of the Microtubule Associate Protein Tau that are expressed by the Peripheral Nervous System and also measuring the absolute quantity of the Total amount of the Microtubule Associate Protein Tau by measuring of at least one peptide that is common to substantially all of the subtypes of Microtubule Associate Protein Tau and then subtracting the quantity of the Tau expressed by the Peripheral Nervous System from the quantity of Total Tau in a method comprising the steps:
   a. isolating Tau proteins and the reference peptides or polypeptides from the sample; then incubating the isolated target proteins and reference peptides or precursor reference peptides or reference polypeptides with a protease to form a target proteolytic digest; or
   b. incubating the sample with a protease to form a proteolytic digest of proteins and reference peptides or precursor reference peptides or reference polypeptides in the sample; and then isolating the target peptides and reference peptides from the proteolytic digest of the sample to form a target proteolytic digest;
ii. assaying the target proteolytic digest of step (i) for the presence of at least one target proteolytic peptide selected from a panel of peptides derived from the subtypes of the Microtubule Associate Protein Tau that are expressed by the Peripheral Nervous System and at least one target proteolytic peptide selected from a panel of peptides that are common to substantially all the subtypes of the Microtubule Associate Protein Tau, wherein the presence of said at least one target proteolytic peptide(s) is quantified using mass spectrometry with reference to the corresponding reference proteolytic peptide or polypeptide; and
iii. diagnosing and/or classifying the severity of the neurodegenerative disease of the subject according to the relative concentration of at least one target proteolytic peptide measured in the sample.

In a first embodiment of the third aspect of the invention, the proteolytic digest is generated by contacting the sample with the protease Trypsin or Trypsin combined with LysC.

In the first embodiment of the third aspect of the invention where the protease is selected from Trypsin or Trypsin combined with LysC, preferably, the assaying step (ii) assays for the presence of at least one target proteotypic peptide derived from the subtypes of the Microtubule Associate Protein Tau that are expressed by the Peripheral Nervous System to be assayed from the target proteolytic digest comprises at least one target proteolytic peptide selected from the group of peptides consisting of:

(SEQ ID NO: 6)
QPSGTGPEDTEGGR
(SEQ ID NO: 7)
DVDESSPQDSPPSK
(SEQ ID NO: 8)
ASPAQDGRPPQTAAR
(SEQ ID NO: 9)
EATSIPGFPAEGAIPLPVDFLSK
(SEQ ID NO: 10)
VSTEIPASEPDGPSVGR
(SEQ ID NO: 11)
GQDAPLEFTFHVEITPNVQK
(SEQ ID NO: 12)
GPSLGEDTK
(SEQ ID NO: 13)
EADLPEPSEK
(SEQ ID NO: 16)
VVQEGFLR
(SEQ ID NO: 17)
HAPELLK
(SEQ ID NO: 18)
HQLLGDLHQEGPPLK
(SEQ ID NO: 19)
EQAHSEEHLGR
(SEQ ID NO: 20)
AAFPGAPGEGPEAR
(SEQ ID NO: 24)
HVPGGGSVQIVYKPVDLSK

In the first embodiment of the third aspect of the invention where the protease is selected from Trypsin or Trypsin combined with LysC, preferably, the assaying step (ii) assays for the presence of at least one target proteotypic peptide that is common to substantially all the subtypes of the Microtubule Associate Protein Tau to be assayed from the target proteolytic digest comprises at least one target proteolytic peptide selected from the group of peptides consisting of:

(SEQ ID NO: 14)
LQTAPVPMPDLK
(SEQ ID NO: 15)
TDHGAEIVYK
(SEQ ID NO: 21)
GAAPPGQK
(SEQ ID NO: 22)
GQANATR
(SEQ ID NO: 23)
HQPGGGK

In a second embodiment of the third aspect of the invention, the proteolytic digest is generated by contacting the sample with the proteases Trypsin combined with AspN or a combination of Trypsin, Lys-C and AspN.

In the second embodiment of the third aspect of the invention where the protease is selected from Trypsin combined with AspN or a combination of Trypsin, Lys-C and AspN, preferably, the assaying step (ii) assays for the presence of at least one target proteotypic peptide derived from the subtypes of the Microtubule Associate Protein Tau that are expressed by the Peripheral Nervous System to be assayed from the target proteolytic digest comprises at least one target proteolytic peptide selected from the group of peptides consisting of:

(SEQ ID NO: 16)
VVQEGFLR
(SEQ ID NO: 17)
HAPELLK
(SEQ ID NO: 18)
HQLLGDLHQEGPPLK
(SEQ ID NO: 19)
EQAHSEEHLGR
(SEQ ID NO: 20)
AAFPGAPGEGPEAR
(SEQ ID NO: 27)
DEAAGHVTQEPESGK
(SEQ ID NO: 28)
DTEGGR
(SEQ ID NO: 29)
DLHQEGPPLK
(SEQ ID NO: 30)
DGRPPQTAAR
(SEQ ID NO: 31)
DGPSVGR
(SEQ ID NO: 32)
DAPLEFTFHVEITPNVQK
(SEQ ID NO: 33)
DLPEPSEK

In the second embodiment of the third aspect of the invention where the protease is selected from Trypsin combined with AspN or a combination of Trypsin, Lys-C and AspN, preferably, the assaying step (ii) assays for the presence of at least one target proteotypic peptide that is common to substantially all the subtypes of the Microtubule Associate Protein Tau to be assayed from the target proteolytic digest comprises at least one target proteolytic peptide selected from the group of peptides consisting of:

(SEQ ID NO: 21)
GAAPPGQK
(SEQ ID NO: 22)
GQANATR
(SEQ ID NO: 23)
HQPGGGK
(SEQ ID NO: 24)
HVPGGGSVQIVYKPVDLSK
(SEQ ID NO: 34)
DNITHVPGGGNK
(SEQ ID NO: 35)
DHGAEIVYK
(SEQ ID NO: 36)
DEVSASLAK

All the features of the first and second aspects of the present invention apply mutatis mutandis to the third aspect of the present invention and vice versa.

Preferably in both the second and third aspects of the invention, the step of preparing a biological sample from the subject by isolating target proteins from the sample includes enrichment of the target proteins. Preferably, the enrichment of the target proteins is carried out by contacting the biological sample from the subject with a solid phase support wherein the support is coated with protein affinity ligands with a binding affinity for the target proteins such that the target proteins bind to the solid phase support allowing extraneous sample material to be washed away. Preferably, the solid phase support is a magnetic microparticle or nanoparticle.

Preferably in all aspects of the invention a target proteotypic peptide derived from the subtypes of the Microtubule Associate Protein Tau that are expressed by the Peripheral Nervous System comprises an amino acid sequence encoded by exon 4a, such as two or more consecutive amino acids or three or more consecutive amino acids encoded by exon 4a.

Preferably in all aspects of the invention a target proteotypic peptide derived from the subtypes of the Microtubule Associate Protein Tau that are expressed by the Central Nervous System does not comprises an amino acid sequence encoded by exon 4a. Preferably a target proteotypic peptide derived from the subtypes of the Microtubule Associate Protein Tau that are expressed by the Central Nervous System comprises the sequence DEAAGHVTQAR (SEQ ID NO: 25) and/or the sequence VQIVYKPV (SEQ ID NO: 26). A target proteotypic peptide derived from the subtypes of the Microtubule Associate Protein Tau that are expressed by the Central Nervous System may comprise the sequence EAGIGDTPSLEDEAAGHVTQAR (SEQ ID NO: 5).

Preferred protein affinity ligands are selected from an Immunoglobulin G antibody, an aptamer, a peptide, an affibody, a small molecule ligand.

Preferably the reference peptides or polypeptides are recombinant versions of the target proteins.

Alternatively, it may be preferred that the reference peptides or polypeptides are synthetic peptides.

Typically and preferably, the reference peptides or polypeptides are modified to incorporate stable heavy isotopes, wherein the stable heavy isotopes are selected from ²H, ¹³C ¹⁵N or ¹⁸O.

Preferably the biological sample from the subject is Whole Blood, Blood Plasma, Serum or Saliva.

All the features of the first and first second aspects of the present invention apply mutatis mutandis to the third aspect of the present invention and vice versa.

In a fourth aspect of the invention, a method is provided for diagnosing and/or classifying the severity of a neurodegenerative disease in a subject, the method comprising:
i. Determining the absolute quantity of Central Nervous System Tau according the methods herein, e.g. first, second and third aspects of the invention.
ii. diagnosing and/or classifying the severity of the neurodegenerative disease of the subject according to the absolute Central Nervous System Tau.

All the features of the first, second and third aspects of the present invention apply mutatis mutandis to the fourth aspect of the present invention and vice versa.

In a fifth aspect of the invention, a method is provided for the treatment of a subject with a Neurodegenerative disease, the method comprising:
i. Determining the absolute quantity of subtypes of Microtubule Associated Protein Tau Expressed by the Central Nervous System in a biological sample from the subject;
ii. diagnosing and/or classifying the severity of the neurodegenerative disease of the subject according to the absolute amount of Central Nervous System Tau measured in the sample; and
iii. treating the subject in need thereof with a therapeutic agent or treatment according to the severity of the Neurodegenerative disease detected in the subject.

The absolute quantity of subtypes of Microtubule Associated Protein Tau Expressed by the Central Nervous System can be determined by any of the methods described herein.

In a sixth aspect of the invention, a method is provided for assessing the efficacy of a treatment for a neurodegenerative disease in a subject, the method comprising:
i. determining the absolute quantity of subtypes of Microtubule Associated Protein Tau Expressed by the Central Nervous System in a biological sample from the subject before treating the subject with a therapeutic agent;
ii. treating the subject in need thereof with a therapeutic agent
iii. determining at one or more further timepoints, after treatment of the subject with a therapeutic agents, the absolute quantity of subtypes of Microtubule Associated Protein Tau Expressed by the Central Nervous System in a biological sample from the subject before treating the subject with a therapeutic agent;
iv. assessing the effectiveness of the treatment based on whether the levels of Microtubule Associated Protein Tau Expressed by the Central Nervous System in the biological samples are reduced.

In the fifth and sixth aspects of the invention, the treatment for the neurodegenerative disease may comprise the administration of a pharmaceutical composition comprising a pharmaceutically active substance. The administration of the pharmaceutical composition may be part of a prescribed course of treatment or a clinical trial. The pharmaceutical composition may be administered once a day, twice a day, once a week, once a month, or at other frequencies according to the course of treatment prescribed or the clinical trial protocol. The clinical trial may be for one month, two months, 6-months or 12 months.

In the first to sixth aspects of the invention, the method may be applied to multiple biological samples from a subject where the biological samples have been obtained at different times. The measurement of the absolute quantity of subtypes of Microtubule Associated Protein Tau Expressed by the Central Nervous System at different times points allows the rate of change or ‘velocity’ of CNS Tau to be determined which may be diagnostically useful as discussed in more detail below.

In a seventh aspect of the invention, a kit is provided for diagnosing and/or classifying the severity of a Neurodegenerative disease in a subject, comprising:
a plurality of sample preparation media for analysis of a biological sample from a subject by mass spectrometry for the presence of at least one target proteolytic peptide selected from the group of peptides from subtypes of Microtubule Associated Protein Tau Expressed by the Central Nervous System, subtypes of Microtubule Associated Protein Tau Expressed by the Peripheral Nervous System and peptides that are common to all subtypes of Microtubule Associated Protein Tau.

In an eighth aspect of the invention, a kit is provided for use in the treatment of a subject with a Neurodegenerative disease or for assessing the efficacy of a treatment for a neurodegenerative disease in a subject, wherein the kit comprises, wherein the kit comprises:
a plurality of sample preparation media for analysis of a biological sample from a subject by mass spectrometry for the presence of at least one target proteolytic peptide selected from the group of peptides from subtypes of Microtubule Associated Protein Tau Expressed by the Central Nervous System, subtypes of Microtubule Associated Protein Tau Expressed by the Peripheral Nervous System and peptides that are common to all subtypes of Microtubule Associated Protein Tau; and a therapeutic agent for treatment of a Neurodegenerative disease.

Preferably, the kits according to the seventh and eighth aspects of the invention further comprise one or more proteases selected from the group consisting of Trypsin or a combination of Trypsin and Endoproteinase Lys-C, Trypsin and Asp-N or a combination of all three of Trypsin and Endoproteinase Lys-C and AspN.

Preferably, the kits according to the seventh and eighth aspects of the invention further comprise reference peptides or polypeptides, such as the reference polypeptides and polypeptides described herein for use in the methods for determining the amount of Tau.

Preferably the reference peptides or polypeptides are recombinant versions of the target proteins or synthetic peptides. Preferably, the reference peptides or polypeptides comprise stable heavy isotopes selected from ¹³C, ¹⁵N, ²H or ¹⁸O.

Preferably, the kits according to the seventh and eighth aspects of the invention additionally comprise solid phase supports derivatised with Protein Affinity Ligands, where there is at least one solid phase support with one Protein Affinity Ligand that binds to the Microtubule-Associated Protein Tau (MAPT). A preferred Protein Affinity Ligand will bind to an epitope that is common to all subtypes of MAPT so that all subtypes are isolated together. Multiple Protein Affinity Ligands that bind to individual subtypes may also be used as long as all the subtypes of MAPT are pulled down together to enable accurate quantification of Central Nervous System Tau.

Preferably also, the kits according to the seventh and eighth aspects of the invention additionally contain machine-readable media containing mass spectrometric transitions or target mass spectra for the target peptides.

Methods are provided for quantifying the amount of subtypes of Tau protein such as Tau protein produced by the Peripheral Nervous System or Tau protein produced by the Central Nervous System. A predetermined amount of reference peptides or polypeptides is added to the sample prior to assaying for the presence of at least one target proteolytic peptide which is substantially unique to the subtype of Tau. Preferably the reference peptides or polypeptides are added to the sample prior to incubation with one or more proteases. The sample is incubated with one or more proteases to form a proteolytic digest of proteins and optionally reference peptides or polypeptides in the sample. The Tau proteins and the reference peptides or polypeptides can be isolated from the sample before the sample is incubated with a protease or after the proteolytic digest has been formed. The proteolytic digest is assayed for the presence of at least one target proteolytic peptide derived from the subtype of the Microtubule Associate Protein Tau, wherein the presence of said at least one target proteolytic peptide(s) is assayed for using mass spectrometry with reference to the corresponding reference proteolytic peptide or polypeptide. Suitable target proteolytic peptides include one or more, such as 2, 3, 4, 5, 6, 7 or 8 peptides specific to the subtype selected from one or more of Table 1, Table 2 and Table 3. Suitable target proteolytic peptides include one or more, such as 2, 3, 4, 5, 6, 7, 8 peptides or all of the peptides specific to the subtype selected from one or more of Table 13, Table 14, Table 16, Table 17, Table 18 and Table 20. The selection of protease and corresponding target proteolytic peptides described herein for other aspects of the invention can also be used in the methods for quantifying the amount of subtypes of Tau protein such as Tau protein produced by the Peripheral Nervous System and Tau protein produced by the Central Nervous System by mass spectrometry. Suitable reference peptides include one or more peptides from Table 5, and/or one or more polypeptides from Table 7, and/or one or more peptides from Table 8. A suitable reference peptide can comprise an amino acid sequence selected from SEQ ID NO: 43 to SEQ ID NO: 78, optionally without a heavy amino acid. Preferably, a precursor reference peptide or reference polypeptide is used to act as a digestion control. A suitable precursor reference peptide can comprise or consist of an amino acid sequence selected from SEQ ID NO: 43 to SEQ ID NO: 186, preferably from SEQ ID NO: 79 to SEQ ID NO: 186, optionally without a heavy amino acid. A suitable reference polypeptide can comprise a polypeptide selected from Table 7.

In aspects of the invention where the target proteins in the sample are isolated before digestion, preferably the reference peptides or polypeptides are recombinant versions of the target proteins. Preferably, the reference proteolytic peptide or polypeptide is derived from digestion of a recombinant heavy protein substantially comprising the sequence of the target protein that includes at a minimum the sequences of the target proteolytic peptides of the assay. Exemplary reference polypeptides are listed in Table 5.

In aspects of the invention where the target proteins in the sample are digested before isolation of peptides from the target proteins, the reference peptides or polypeptides are preferably either recombinant versions of the target proteins or synthetic peptides. When synthetic reference peptides are used, preferably the peptides comprise the same sequence as the target peptides of the assay. Preferably, the synthetic peptides additionally comprise a flanking sequence at either or both termini of the peptide to act as a digestion control.

Preferably, the reference peptides or polypeptides have been modified to comprise stable heavy isotopes, typically selected from ¹³C, ¹⁵N, ²H or ¹⁸O. Exemplary reference peptides or polypeptides are listed in Table 8.

In aspects of the invention, preferably, the step of preparing a biological sample includes enrichment of the target proteins from the subject by isolating proteins from the sample prior to digestion of the sample proteins. More specifically, the enrichment of the target proteins may be carried out by contacting the biological sample from the subject with a solid phase support wherein the support is coated with protein affinity ligands with a binding affinity for the target proteins such that the target proteins bind to the solid phase support allowing extraneous sample material to be washed away. Preferably, the step of isolating proteins from the sample is effected using immuno-precipitation to enrich for the target proteins and to allow the background matrix to be removed.

In aspects of the invention, the step of preparing a biological sample from the subject may additionally include enrichment of target peptides when the method involves isolating target peptides from the target proteins in the sample after digestion of the sample with a protease. The enrichment of the target peptides may be carried out by contacting the biological sample from the subject with a solid phase support, wherein the support is coated with protein affinity ligands with a binding affinity for the target peptides, such that the target peptides bind to the solid phase support, allowing extraneous sample material to be washed away. This method differs from the method where proteins are isolated from the sample before proteolytic digestion in two ways: the proteins are digested before enrichment of peptides and the protein affinity ligands must recognise the tryptic peptides as their epitope. In the following disclosure, we will refer to protein affinity ligands that have a specific affinity for a linear peptide epitope as peptide affinity ligands. Exemplary Peptide Affinity Ligands are listed in Table 6.

More specifically, protein or peptide affinity ligands such as monoclonal antibodies, polyclonal antibodies, affibodies, aptamers, peptides, small molecule ligands or other protein or peptide affinity ligands that selectively bind to the peptides or proteins in the panel of target proteins are immobilised on a solid phase support and the solid phase support is contacted with the biological sample under conditions that promote binding of the target proteins or digested peptides to the solid support. Material that is not bound to the solid support may be washed away leaving an enriched sample containing the target proteins or digested target peptides from the target proteins. The target proteins or target peptides may then be eluted from the solid support before step (ii), where the peptides are analysed to identify target proteins. When proteins are eluted from the solid support, the proteins are then incubated with a protease. Alternatively, proteolysis may take place while the target proteins remain on the solid support and the support is then contacted with the protease to digest the target proteins off the support.

Preferably, the solid phase support is a magnetic microparticle or nanoparticle.

Preferably, the protein affinity ligand is selected from an Immunoglobulin G antibody, an aptamer, a peptide, an affibody, a small molecule ligand.

In all aspects of the invention, the biological sample from a subject is preferably whole blood, plasma or serum. Other preferred samples include Cerebrospinal Fluid, Urine, Sweat and Saliva.

As a sample type, plasma is also challenging to prepare for mass spectrometry applications.

However, in the present invention, samples based on plasma have been used successfully.

The plasma sample preparation in the methods of the present invention avoids problems of the prior art (analytical signal suppression and variability due to high abundance proteins such as albumin) by enriching the target proteins or peptides by Immuno-Precipitation and by selecting the optimal peptides for analysis with corresponding heavy isotope labelled-internal standards with digestion efficiency control tags and calibration curves. Without wishing to be bound by theory, whole blood, plasma or serum samples (specifically from venous blood) may be the most suitable form of sample for use in a method of the present invention along with Urine, Saliva Sweat or CSF.

Preferably, according to any of the aspects of the invention, the Neurodegenerative disease comprises Alzheimer's disease or another tauopathy. The neurodegenerative disease may also comprise other tauopathies or tau-related disorders and pathologies including Pick disease, progressive supranuclear palsy, corticobasal degeneration, argyrophilic grain disease, globular glial tauopathies, primary age-related tauopathy, including neurofibrillary tangle dementia, chronic traumatic encephalopathy (CTE), and aging-related tau astrogliopathy.

Preferably, the neurodegenerative disease is Alzheimer's disease.

TABLE 1
Target Proteins and Target Tryptic Peptides with Aspartic Acid:

| Tau | | | Peptide | |
|---|---|---|---|---|
| Species | Biomarker Name | Uniprot ID | Mass | Sequence |
| CNS-Tau | Microtubule-Associated | A0A024R9Y0 | 2423.1200 | AEEAGIGDTPSLEDEAAGHVTQAR |
| | Protein | | | (SEQ ID NO: 1) |
| | Microtubule-Associated Protein | A0A7POT936 | | |
| | Isoform Fetal-tau of Microtubule- | P10636-2 | | |
| | Associated Protein Tau | | | |
| | Isoform Tau-D of Microtubule- | P10636-6 | | |
| | associated Protein Tau | | | |
| | (Tau-383; ON4R) | | | |
| | Microtubule-Associated | A0A024RA17 | 3954.8467 | QAAAQPHTEIPEGTTAEEAGIGD |
| | Protein | | | TPSLEDEAAGHVTQAR |
| | Isoform Tau-C of Microtubule- | P10636-5 | | (SEQ ID NO: 2) |
| | Associated Protein Tau | | | |
| | (Tau-410; 2N3R) | | | |
| | Isoform Tau-F of Microtubule- | P10636-8 | | |
| | Associated Protein Tau | | | |
| | (Tau-441; 2N4R) | | | |
| | Microtubule-Associated | A0A024RA19 | 3009.3799 | STPTAEAEEAGIGDTPSLEDEAAG |
| | Protein | | | HVTQAR |
| | Isoform Tau-B of Microtubule- | P10636-4 | | (SEQ ID NO: 3) |
| | Associated Protein Tau | | | |
| | (Tau-381; 1N3R) | | | |
| | Isoform Tau-E of Microtubule- | P10636-7 | | |
| | Associated Protein Tau | | | |
| | (Tau-412; 1N4R) | | | |
| | Microtubule-Associated | A0A024RA19 | 1387.8075 | VQIVYKPVDLSK |
| | Protein | | | (SEQ ID NO: 4) |
| | Microtubule-Associated Protein | 13L170 | | |
| | Isoform Fetal-tau of Microtubule- | P10636-2 | | |
| | Associated Protein Tau | | | |
| | Isoform Tau-A of Microtubule- | P10636-3 | | |
| | Associated Protein Tau (ON3R) | | | |
| | Isoform Tau-B of Microtubule- | P10636-4 | | |
| | Associated Protein Tau | | | |
| | (Tau-381; 1N3R) | | | |
| | Isoform Tau-C of Microtubule- | P10636-5 | | |
| | Associated Protein Tau | | | |
| | (Tau-410; 2N3R) | | | |
| | Microtubule-Associated Protein | A0A712PLE3 | | |
| | Microtubule-Associated Protein | B3KTM0 | | |
| | Microtubule-Associated Protein | B4DSB3 | | |
| | Microtubule-Associated Protein | B4DSE3 | | |
| | Isoform Tau-A of Microtubule- | P10636-3 | 2223.0403 | EAGIGDTPSLEDEAAGHVTQAR |
| | Associated Protein Tau (ON3R) | | | (SEQ ID NO: 5) |
| PNS-Tau | Microtubule-Associated Protein | P10636; | 1386.6011 | QPSGTGPEDTEGGR |
| | Tau Exon 4a (Big Tau) | P10636-9; | | (SEQ ID NO: 6) |
| | | A0A024R9Y1; | 1486.6423 | DVDESSPQDSPPSK |
| | | A0A0G2JPD5; | | (SEQ ID NO: 7) |
| | | A0A0G2JQJ7; | 1521.7648 | ASPAQDGRPPQTAAR |
| | | A0A0G2JS76; | | (SEQ ID NO: 8) |
| | | A0A712PJZ2; | | |
| | | A0A712PLE3; | 1696.8268 | VSTEIPASEPDGPSVGR |
| | | B7U472 | | (SEQ ID NO: 10) |
| | | | 2269.1379 | GQDAPLEFTFHVEITPNVQK |
| | | | | (SEQ ID NO: 11) |
| | | | 902.4345 | GPSLGEDTK |
| | | | | (SEQ ID NO: 12) |
| | | | 1113.5190 | EADLPEPSEK |
| | | | | (SEQ ID NO: 13) |
| Total-Tau | All variants | All variants | 1308.7111 | LQTAPVPMPDLK |
| | | | | (SEQ ID NO: 14) |
| | | | 1131.5560 | TDHGAEIVYK |
| | | | | (SEQ ID NO: 15) |
| | | | 1979.0840 | HVPGGGSVQIVYKPVDLSK |
| | | | | (SEQ ID NO: 24) |
| | | | 1577.8161 | IGSLDNITHVPGGGNK |
| | | | | (SEQ ID NO: 187) |

TABLE 2
Target Proteins and Target Double Cleavage Peptides
that are shared with Tryptic Digest:

| Tau | | | Peptide | |
|---|---|---|---|---|
| Species | Biomarker Name | Uniprot ID | Mass | Sequence |
| PNS-Tau | Microtubule- | P10636; | 946.5236 | VVQEGFLR (SEQ ID NO: 16) |
| | Associated | P10636-9; | 806.4650 | HAPELLK (SEQ ID NO: 17) |
| | Protein Tau Exon | A0A024R9Y1; | 1680.8947 | HQLLGDLHQEGPPLK (SEQ ID NO: 18) |
| | 4a (Big Tau) | A0A0G2JMX7; | 1291.5905 | EQAHSEEHLGR (SEQ ID NO: 19) |
| | | A0A0G2JPD5; | 1325.6364 | AAFPGAPGEGPEAR (SEQ ID NO: 20) |
| | | A0A0G2JQJ7; | | |
| | | A0A0G2JS76; | | |
| | | A0A712PJZ2; | | |
| | | A0A712PLE3; | | |
| | | B7U472 | | |
| Total-Tau | All variants | All variants | 724.3868 | GAAPPGQK (SEQ ID NO: 21) |
| | | | 716.3565 | GQANATR (SEQ ID NO: 22) |
| | | | 679.3402 | HQPGGGK (SEQ ID NO: 23) |

TABLE 3
Target Proteins and Target Double Cleavage Peptides according
to this invention unique to Asp-N/Trypsin Digest:

| Tau | | | Peptide | |
|---|---|---|---|---|
| Species | Biomarker Name | Uniprot ID | Mass | Sequence |
| CNS-Tau | Microtubule-Associated | All CNS | 1153.5476 | DEAAGHVTQAR (SEQ ID NO: 25) |
| | Protein Tau | Variants | 944.5695 | VQIVYKPV (SEQ ID NO: 26) |
| PNS-Tau | Microtubule- | P10636; | 1553.6957 | DEAAGHVTQEPESGK (SEQ ID NO: 27) |
| | Associated Protein | P10636-9; | 633.2718 | DTEGGR (SEQ ID NO: 28) |
| | Tau Exon 4a (Big Tau) | A0A024R9Y1; | 1132.5876 | DLHQEGPPLK (SEQ ID NO: 29) |
| | | A0A0G2JMX7; | 1067.5472 | DGRPPQTAAR (SEQ ID NO: 30) |
| | | A0A0G2JPD5; | 686.3347 | DGPSVGR (SEQ ID NO: 31) |
| | | A0A0G2JQJ7; | 2084.0578 | DAPLEFTFHVEITPNVQK (SEQ ID NO: 32) |
| | | A0A0G2JS76; | 913.4393 | DLPEPSEK (SEQ ID NO: 33) |
| | | A0A712PJZ2; | | |
| | | A0A712PLE3; | | |
| | | B7U472 | | |
| Total-Tau | All variants | All variants | 1207.5945 | DNITHVPGGGNK (SEQ ID NO: 34) |
| | | | 1030.5083 | DHGAEIVYK (SEQ ID NO: 35) |
| | | | 918.4658 | DEVSASLAK (SEQ ID NO: 36) |
| | | | 1535.8460 | HVPGGGSVQIVYKPV (SEQ ID NO: 188) |

The methods of the present invention use mass spectrometry to identify the concentrations of target peptides, for example proteolytic peptides having the sequences as set out in Tables 1, 2 and 3. The methods of the present invention provide for the detection of up to 41 proteolytic peptides from different subtypes of MAPT as shown in Tables 1, 2 and 3. The concentrations of the peptides may be used to diagnose and/or classifying the severity of neurodegenerative disease in a subject (and to treat neurodegenerative disease in a subject and to assess the efficacy of a treatment for a neurodegenerative as described further below), with reference to a calibration curve of the concentrations for known reference proteolytic peptides.

The concentrations of the proteolytic peptides assayed for in the samples are therefore linked with diagnosing and/or classifying the severity of neurodegenerative disease in a subject, treating a neurodegenerative disease in a subject and assessing the efficacy of a treatment for a neurodegenerative disease. Generally, an increase in the proteolytic peptide concentration of peptides from subtypes of MAPT expressed by the Central Nervous System from a potential neurodegenerative disease patient compared to a healthy control is indicative of a diagnosis of a neurodegenerative disease, and the relative increase indicates the severity of the neurodegenerative disease. Conversely, decreases or stabilisation of the concentration of peptides from subtypes of MAPT expressed by the Central Nervous System after administration of a treatment may indicate the efficacy of the treatment for a neurodegenerative disease.

The control may be a baseline control. The control may be a placebo group. The concentrations of the proteolytic peptides may be normalised to the control. Concentrations may be expressed as fold changes relative to the control, such as fold changes from baseline.

The methods of the invention may further account for whether the proteolytic peptides are shown herein to be up-regulated or downregulated. Biomarker levels across different patients groups, for instance in a clinical trial, may be different; i.e. increased or decreased relative to another group (such as a control or a placebo). A combination of biomarkers may be used to separate patient groups based on biomarker concentration and using a principal component analysis (PCA) or fold change differences. Fold change differences may be used to separate patient groups using a threshold or cut-off point.

The treatment for the neurodegenerative disease may comprise the administration of a pharmaceutical composition comprising a pharmaceutically active substance. The administration of the pharmaceutical composition may be part of a prescribed course of treatment or a clinical trial. The pharmaceutical composition may be administered once a day, twice a day, once a week, once a month, or at other frequencies according to the course of treatment prescribed or the clinical trial protocol. The clinical trial may be for one month, two months, 6-months or 12 months.

The efficacy of a treatment may be a measure of the effectiveness of any generally suitable method treatment for the neurodegenerative disease. The efficacy may be expressed in terms of the presence or absence of one or more clinical symptoms or one or more side-effects of the pharmaceutical composition used to treat the subject. The efficacy of a treatment may also therefore be correlated with a change in the concentration of a protein biomarker in a sample comprising a protein as shown in Tables 1, 2 or 3. The change in the concentration of a relevant biomarker as defined herein may be an increase in concentration of a biomarker or a decrease in concentration of a biomarker compared to a baseline or control value for such a biomarker.

Absolute peptide concentration may be compared across a set of proteins in a sample. The samples may be collected at different time intervals from the start of treatment regime (i.e. time at Week 0 (“Week zero”). Subsequent samples may be collected from patients at approximately Week 20 and/or Week 45. The results for the samples obtained at different time points can then be normalised to a baseline and the results can be expressed as relative change from the baseline. For example, a 1.5-fold peptide concentration increase or 0.67-fold concentration decrease from baseline in drug treated groups whilst having no or lesser corresponding peptide alteration in the placebo group may be considered to indicate drug treatment efficacy for a pharmaceutical composition comprising a drug under assessment according to a method of the invention.

Subsequent samples may be collected from patients at approximately Week 2, 4, 6, 8, 10, 12, 14, 16, 18, 20, 22, 24, 26, 28 or 30 and/or Week 35, 40, 45, 50, 55 or 60.

Subsequent samples may be collected from patients at approximately month 1, 2, 3, 4, 5 or 6 and/or month 7, 8, 9, 10, 11 or 12. Subsequent samples may be collected from patients at approximately six-month intervals. Subsequent samples may be collected from patients at approximately month 6 and/or month 12.

An at least 1.5-fold peptide concentration increase and/or an at least 0.67-fold concentration decrease from baseline in drug treated groups whilst having no or lesser corresponding peptide alteration in the placebo group may be considered to indicate drug treatment efficacy for a pharmaceutical composition comprising a drug under assessment according to a method of the invention.

An at least 1.1, at least 1.2, at least 1.3, at least 1.4, at least 1.5, at least 1.6, at least 1.7, at least 1.8, at least 1.9 or at least 2.0-fold peptide concentration increase and/or an at least 0.9, at least 0.8, at least 0.7, at least 0.6, at least 0.67, at least 0.5, at least 0.4, at least 0.3, at least 0.2 or at least 0.1-fold concentration decrease from baseline in drug treated groups whilst having no or lesser corresponding peptide alteration in the placebo group may be considered to indicate drug treatment efficacy for a pharmaceutical composition comprising a drug under assessment according to a method of the invention.

The invention also provides a computer-implemented method for assessing the efficacy of a treatment for a neurodegenerative disease in a subject, which comprises receiving in a computer sample data representing the level of at least one proteolytic peptide in a biological sample obtained from a subject and executing software on the computer to compare the level of the at least at least one proteolytic peptide in the sample to a baseline control, wherein the difference between the level of the at least one proteolytic peptide and the baseline control is indicative of the efficacy of a treatment for a neurodegenerative disease, and to output efficacy data representing the efficacy of a treatment for a neurodegenerative disease on the basis of the comparison.

The invention also provides a computer program comprising instructions which, when executed by a computer, cause the computer to carry out a computer implemented method of the invention.

It will be appreciated that the step of comparing the level of the at least at least one proteolytic peptide in the sample with a baseline control may be carried out on a different computer from a computer that initially receives data representing the at least at least one proteolytic peptide in the sample.

The invention also provides a computer apparatus for assessing the efficacy of a treatment for a neurodegenerative disease, which comprises a first device incorporating a computer, a second computer and a communication channel between the first device and second computer for the transmission of data therebetween; wherein the first device is arranged to receive sample data representing level of the at least one proteolytic peptide in a sample obtained from the subject and to transmit the sample data to the second computer via the communication channel, and the second computer is arranged to execute software to compare levels of the at least at least one proteolytic peptide in the sample to a baseline control to determine the efficacy of a treatment for a neurodegenerative disease, wherein the difference between the level of the at least one proteolytic peptide and the baseline control is indicative of the efficacy of a treatment for a neurodegenerative disease, and to output efficacy data representing the efficacy of a treatment for a neurodegenerative disease on the basis of the comparison.

The second computer may be arranged to transmit the efficacy data to the first device via the communication channel, or to a third computer.

The first device may incorporate mass spectrometry equipment or devices for measuring the level of at least one proteolytic peptide in a sample.

The present invention also provides kits suitable for carrying out the methods of the present invention.

The kit may be suitable for diagnosing and/or classifying the severity of the neurodegenerative disease using a method according to the first aspect of the present invention.

All the features of the first, second and third aspects of the present invention apply mutatis mutandis to the fourth aspect of the present invention and vice versa.

Optionally, the kit may additionally comprise a biological sample collection device.

Suitable media may comprise one or more of the following selected from the group consisting of a peptide storage solvent (for example comprising aqueous acetonitrile, optionally comprising 50% acetonitrile/50% double deionized H₂O (ddH₂O)), a denaturation buffer (for example comprising urea, ammonium bicarbonate and dithiothreitol (DTT), optionally comprising 8M Urea, 100 mM ammonium bicarbonate, 50 mM DTT); an alkylation agent (for example comprising 2-iodoacetamide (IAA), optionally comprising 100 mM IAA); salts (for example comprising triethylammonium bicarbonate (TEAB), optionally comprising 100 mM TEAB), and/or a surrogate matrix (for example bovine serum albumin (BSA), optionally comprising 40 mg/ml bovine serum albumin in ddH2O).

In the sixth and seventh aspects of this invention, the kits may additionally contain machine-readable media containing mass spectrometric transitions or target mass spectra for the target peptides in Tables 1, 2 and 3. The transitions or target mass spectra on the machine-readable media may be used by a mass spectrometer to identify and quantify the target peptides in patient samples. Preferred transitions for use in this invention are listed in Table 4 below. It should be obvious to one of ordinary skill in the art that the product ion data for these transitions may also be used to confirm the identity of target peptides in spectra obtained by Parallel Reaction Monitoring.

In the sixth and seventh aspects of this invention, the kit may also comprise instructions for use.

TABLE 4
Preferred Transitions for Target Peptides:

| | | | | Product | |
|---|---|---|---|---|---|
| | | Precursor | Product | Ion | Product |
| | Precursor | Ion Charge | Ion | Charge | Ion |
| Peptide Sequence | Ion m/z | State | m/z | State | Type |
| AEEAGIGDTPSLEDEAAGHVTQAR (SEQ ID NO: 1) | 808.7139 | 3 | 201.0870 | 1/2 | b2+/b4++ |
| AEEAGIGDTPSLEDEAAGHVTQAR (SEQ ID NO: 1) | 808.7139 | 3 | 173.0921 | 1 | a2 |
| AEEAGIGDTPSLEDEAAGHVTQAR (SEQ ID NO: 1) | 808.7139 | 3 | 768.4111 | 1 | y7 |
| AEEAGIGDTPSLEDEAAGHVTQAR (SEQ ID NO: 1) | 808.7139 | 3 | 839.4482 | 1 | Y8 |
| AEEAGIGDTPSLEDEAAGHVTQAR (SEQ ID NO: 1) | 808.7139 | 3 | 910.4853 | 1 | y9 |
| QAAAQPHTEIPEGTTAEEAGIGDTPSLEDEAAGHVT | 1320.2927 | 3 | 1030.4776 | 3 | a31 |
| QAR (SEQ ID NO: 2) | | | | | |
| QAAAQPHTEIPEGTTAEEAGIGDTPSLEDEAAGHVT | 1320.2927 | 3 | 1580.7663 | 1 | y15 |
| QAR (SEQ ID NO: 2) | | | | | |
| QAAAQPHTEIPEGTTAEEAGIGDTPSLEDEAAGHVT | 1320.2927 | 3 | 790.8868 | 2 | y15 |
| QAR (SEQ ID NO: 2) | | | | | |
| QAAAQPHTEIPEGTTAEEAGIGDTPSLEDEAAGHVT | 1320.2927 | 3 | 475.2623 | 1 | y4 |
| QAR (SEQ ID NO: 2) | | | | | |
| QAAAQPHTEIPEGTTAEEAGIGDTPSLEDEAAGHVT | 1320.2927 | 3 | 1047.5218 | 1 | b10 |
| QAR (SEQ ID NO: 2) | | | | | |
| STPTAEAEEAGIGDTPSLEDEAAGHVTQAR (SEQ ID NO: 3) | 1004.1308 | 3 | 1580.7663 | 1 | y15 |
| STPTAEAEEAGIGDTPSLEDEAAGHVTQAR (SEQ ID NO: 3) | 1004.1308 | 3 | 1039.5279 | 1 | y10 |
| STPTAEAEEAGIGDTPSLEDEAAGHVTQAR (SEQ ID NO: 3) | 1004.1308 | 3 | 768.4111 | 1 | y7 |
| STPTAEAEEAGIGDTPSLEDEAAGHVTQAR (SEQ ID NO: 3) | 1004.1308 | 3 | 839.4482 | 1 | y8 |
| STPTAEAEEAGIGDTPSLEDEAAGHVTQAR (SEQ ID NO: 3) | 1004.1308 | 3 | 910.4853 | 1 | y9 |
| VQIVYKPVDLSK (SEQ ID NO: 4) | 695.47 | 2 | 581.3475 | 2 | y10 |
| VQIVYKPVDLSK (SEQ ID NO: 4) | 695.47 | 2 | 949.5353 | 1 | y8 |
| VQIVYKPVDLSK (SEQ ID NO: 4) | 695.47 | 2 | 1048.6037 | 1 | y9 |
| VQIVYKPVDLSK (SEQ ID NO: 4) | 695.47 | 2 | 658.3770 | 1 | y6 |
| VQIVYKPVDLSK (SEQ ID NO: 4) | 695.47 | 2 | 786.4720 | 1 | y7 |
| EAGIGDTPSLEDEAAGHVTQAR (SEQ ID NO: 5) | 1112.5275 | 2 | 910.4853 | 1 | y9 |
| EAGIGDTPSLEDEAAGHVTQAR (SEQ ID NO: 5) | 1112.5275 | 2 | 841.4106 | 2 | y16 |
| EAGIGDTPSLEDEAAGHVTQAR (SEQ ID NO: 5) | 1112.5275 | 2 | 1025.4716 | 2 | b21 |
| EAGIGDTPSLEDEAAGHVTQAR (SEQ ID NO: 5) | 1112.5275 | 2 | 1580.7663 | 1 | y15 |
| EAGIGDTPSLEDEAAGHVTQAR (SEQ ID NO: 5) | 1112.5275 | 2 | 811.8683 | 2 | a17 |
| QPSGTGPEDTEGGR (SEQ ID NO: 6) | 694.3084 | 2 | 917.3965 | 1 | y9 |
| QPSGTGPEDTEGGR (SEQ ID NO: 6) | 694.3084 | 2 | 860.3745 | 1 | y8 |
| QPSGTGPEDTEGGR (SEQ ID NO: 6) | 694.3084 | 2 | 1162.4971 | 1 | y12 |
| QPSGTGPEDTEGGR (SEQ ID NO: 6) | 694.3084 | 2 | 519.2522 | 1 | y5 |
| QPSGTGPEDTEGGR (SEQ ID NO: 6) | 694.3084 | 2 | 289.1619 | 1 | y3 |
| DVDESSPQDSPPSK (SEQ ID NO: 7) | 744.3284 | 2 | 855.4207 | 1 | y8 |
| DVDESSPQDSPPSK (SEQ ID NO: 7) | 744.3284 | 2 | 187.1077 | 1 | a2 |
| DVDESSPQDSPPSK (SEQ ID NO: 7) | 744.3284 | 2 | 428.2504 | 1 | y4 |
| DVDESSPQDSPPSK (SEQ ID NO: 7) | 744.3284 | 2 | 942.4527 | 1 | y9 |
| DVDESSPQDSPPSK (SEQ ID NO: 7) | 744.3284 | 2 | 1029.4847 | 1 | y10 |
| ASPAQDGRPPQTAAR (SEQ ID NO: 8) | 508.2628 | 3 | 634.3287 | 2 | y12 |
| ASPAQDGRPPQTAAR (SEQ ID NO: 8) | 508.2628 | 3 | 682.8557 | 2 | y13 |
| ASPAQDGRPPQTAAR (SEQ ID NO: 8) | 508.2628 | 3 | 598.8102 | 2 | y11 |
| ASPAQDGRPPQTAAR (SEQ ID NO: 8) | 508.2628 | 3 | 455.5725 | 3 | y13 |
| ASPAQDGRPPQTAAR (SEQ ID NO: 8) | 508.2628 | 3 | 534.7814 | 2 | y10 |
| EATSIPGFPAEGAIPLPVDFLSK (SEQ ID NO: 9) | 1178.6254 | 2 | 1015.5823 | 1 | y9 |
| EATSIPGFPAEGAIPLPVDFLSK (SEQ ID NO: 9) | 1178.6254 | 2 | 805.4454 | 1 | y7 |
| EATSIPGFPAEGAIPLPVDFLSK (SEQ ID NO: 9) | 1178.6254 | 2 | 1553.8574 | 1 | y15 |
| EATSIPGFPAEGAIPLPVDFLSK (SEQ ID NO: 9) | 1178.6254 | 2 | 1855.0000 | 1 | y18 |
| EATSIPGFPAEGAIPLPVDFLSK (SEQ ID NO: 9) | 1178.6254 | 2 | 1256.7249 | 1 | y12 |
| VSTEIPASEPDGPSVGR (SEQ ID NO: 10) | 849.4212 | 2 | 1168.5598 | 1 | y12 |
| VSTEIPASEPDGPSVGR (SEQ ID NO: 10) | 566.6168 | 3 | 392.7016 | 2 | y8 |
| VSTEIPASEPDGPSVGR (SEQ ID NO: 10) | 566.6168 | 3 | 572.3156 | 1 | y6 |
| VSTEIPASEPDGPSVGR (SEQ ID NO: 10) | 566.6168 | 3 | 584.7838 | 2 | y12 |
| VSTEIPASEPDGPSVGR (SEQ ID NO: 10) | 566.6168 | 3 | 784.3953 | 1 | y8 |
| GQDAPLEFTFHVEITPNVOK (SEQ ID NO: 11) | 757.3871 | 3 | 301.1143 | 1 | b3 |
| GQDAPLEFTFHVEITPNVQK (SEQ ID NO: 11) | 757.3871 | 3 | 950.0042 | 2 | y16 |
| GQDAPLEFTFHVEITPNVQK (SEQ ID NO: 11) | 757.3871 | 3 | 186.0873 | 1 | b2 |
| GQDAPLEFTFHVEITPNVQK (SEQ ID NO: 11) | 757.3871 | 3 | 585.3355 | 1 | y5 |
| GQDAPLEFTFHVEITPNVQK (SEQ ID NO: 11) | 757.3871 | 3 | 928.5098 | 1 | y8 |
| GPSLGEDTK (SEQ ID NO: 12) | 452.2251 | 2 | 127.0866 | 1 | a2 |
| GPSLGEDTK (SEQ ID NO: 12) | 452.2251 | 2 | 549.252 | 1 | y5 |
| GPSLGEDTK (SEQ ID NO: 12) | 452.2251 | 2 | 749.3681 | 1 | y7 |
| GPSLGEDTK (SEQ ID NO: 12) | 452.2251 | 2 | 248.161 | 1 | y2 |
| GPSLGEDTK (SEQ ID NO: 12) | 452.2251 | 2 | 155.0815 | 1 | b2 |
| EADLPEPSEK (SEQ ID NO: 13) | 557.7673 | 2 | 460.2407 | 1 | y4 |
| EADLPEPSEK (SEQ ID NO: 13) | 557.7673 | 2 | 686.3361 | 1 | y6 |
| EADLPEPSEK (SEQ ID NO: 13) | 557.7673 | 2 | 316.1145 | 1 | b3 |
| EADLPEPSEK (SEQ ID NO: 13) | 557.7673 | 2 | 173.0921 | 1 | a2 |
| EADLPEPSEK (SEQ ID NO: 13) | 557.7673 | 2 | 914.4471 | 1 | y8 |
| VVQEGFLR (SEQ ID NO: 16) | 474.2691 | 2 | 749.3941 | 1 | y6 |
| VVQEGFLR (SEQ ID NO: 16) | 474.2691 | 2 | 171.1492 | 1 | a2 |
| VVQEGFLR (SEQ ID NO: 16) | 474.2691 | 2 | 621.3355 | 1 | y5 |
| VVQEGFLR (SEQ ID NO: 16) | 474.2691 | 2 | 492.2929 | 1 | y4 |
| VVQEGFLR (SEQ ID NO: 16) | 474.2691 | 2 | 175.1190 | 1 | y1 |
| HAPELLK (SEQ ID NO: 17) | 404.243 | 2 | 209.1039 | 1 | b2 |
| HAPELLK (SEQ ID NO: 17) | 404.243 | 2 | 181.1084 | 1 | a2 |
| HAPELLK (SEQ ID NO: 17) | 404.243 | 2 | 147.1128 | 1 | y1 |
| HAPELLK (SEQ ID NO: 17) | 404.243 | 2 | 599.3763 | 1 | y5 |
| HAPELLK (SEQ ID NO: 17) | 404.243 | 2 | 548.2827 | 1 | b5 |
| HQLLGDLHQEGPPLK (SEQ ID NO: 18) | 561.3083 | 3 | 1228.6069 | 2 | b11 |
| HQLLGDLHQEGPPLK (SEQ ID NO: 18) | 561.3083 | 3 | 266.1248 | 1 | b2 |
| HQLLGDLHQEGPPLK (SEQ ID NO: 18) | 561.3083 | 3 | 147.1128 | 1 | y1 |
| HQLLGDLHQEGPPLK (SEQ ID NO: 18) | 561.3083 | 3 | 664.3413 | 1 | b6 |
| HQLLGDLHQEGPPLK (SEQ ID NO: 18) | 561.3083 | 3 | 768.4250 | 1 | y7 |
| EQAHSEEHLGR (SEQ ID NO: 19) | 431.5381 | 3 | 345.225 | 1 | y3 |
| EQAHSEEHLGR (SEQ ID NO: 19) | 431.5381 | 3 | 232.141 | 1 | y2 |
| EQAHSEEHLGR (SEQ ID NO: 19) | 431.5381 | 3 | 827.4006 | 1 | y7 |
| EQAHSEEHLGR (SEQ ID NO: 19) | 431.5381 | 3 | 414.2045 | 2 | y7 |
| EQAHSEEHLGR (SEQ ID NO: 19) | 431.5381 | 3 | 482.7334 | 2 | y8 |
| AAFPGAPGEGPEAR (SEQ ID NO: 20) | 663.826 | 2 | 812.3903 | 1 | y8 |
| AAFPGAPGEGPEAR (SEQ ID NO: 20) | 663.826 | 2 | 519.2547 | 2 | y11 |
| AAFPGAPGEGPEAR (SEQ ID NO: 20) | 663.826 | 2 | 246.1566 | 1 | y2 |
| AAFPGAPGEGPEAR (SEQ ID NO: 20) | 663.826 | 2 | 406.699 | 2 | y8 |
| AAFPGAPGEGPEAR (SEQ ID NO: 20) | 663.826 | 2 | 529.2734 | 1 | y5 |
| LQTAPVPMPDLK (SEQ ID NO: 14) | 655.3634 | 2 | 896.4915 | 1 | y8 |
| LQTAPVPMPDLK (SEQ ID NO: 14) | 655.3634 | 2 | 1068.5758 | 1 | y10 |
| LQTAPVPMPDLK (SEQ ID NO: 14) | 655.3634 | 2 | 700.3704 | 1 | y6 |
| LQTAPVPMPDLK (SEQ ID NO: 14) | 655.3634 | 2 | 472.2771 | 1 | y4 |
| LQTAPVPMPDLK (SEQ ID NO: 14) | 655.3634 | 2 | 967.5281 | 1 | y9 |
| TDHGAEIVYK (SEQ ID NO: 15) | 378.1932 | 3 | 611.2425 | 1 | b6 |
| TDHGAEIVYK (SEQ ID NO: 15) | 378.1932 | 3 | 409.2451 | 1 | y3 |
| TDHGAEIVYK (SEQ ID NO: 15) | 378.1932 | 3 | 310.1767 | 1 | y2 |
| TDHGAEIVYK (SEQ ID NO: 15) | 378.1932 | 3 | 522.3292 | 1 | y4 |
| TDHGAEIVYK (SEQ ID NO: 15) | 378.1932 | 3 | 306.1252 | 2 | b6 |
| GAAPPGQK (SEQ ID NO: 21) | 363.2012 | 2 | 200.1035 | 1 | b3 |
| GAAPPGQK (SEQ ID NO: 21) | 363.2012 | 2 | 526.2989 | 1 | y5 |
| GAAPPGQK (SEQ ID NO: 21) | 363.2012 | 2 | 429.2462 | 1 | y4 |
| GAAPPGQK (SEQ ID NO: 21) | 363.2012 | 2 | 597.336 | 1 | y6 |
| GAAPPGQK (SEQ ID NO: 21) | 363.2012 | 2 | 263.6534 | 2 | y5 |
| GQANATR (SEQ ID NO: 22) | 359.1861 | 2 | 532.2843 | 1 | y5 |
| GQANATR (SEQ ID NO: 22) | 359.1861 | 2 | 276.1672 | 1 | y2 |
| GQANATR (SEQ ID NO: 22) | 359.1861 | 2 | 461.2472 | 1 | y4 |
| GQANATR (SEQ ID NO: 22) | 359.1861 | 2 | 347.2043 | 1 | y3 |
| GQANATR (SEQ ID NO: 22) | 359.1861 | 2 | 257.125 | 1 | b3 |
| HQPGGGK (SEQ ID NO: 23) | 227.4545 | 3 | 266.1253 | 1 | b2 |
| HQPGGGK (SEQ ID NO: 23) | 227.4545 | 3 | 208.1192 | 2 | y5 |
| HQPGGGK (SEQ ID NO: 23) | 227.4545 | 3 | 204.1348 | 1 | y2 |
| HQPGGGK (SEQ ID NO: 23) | 227.4545 | 3 | 210.6037 | 2 | b4 |
| HQPGGGK (SEQ ID NO: 23) | 227.4545 | 3 | 272.1485 | 2 | y6 |
| HVPGGGSVQIVYKPVDLSK (SEQ ID NO: 24) | 660.7025 | 3 | 872.4862 | 2 | y17 |
| HVPGGGSVQIVYKPVDLSK (SEQ ID NO: 24) | 660.7025 | 3 | 237.1352 | 1 | b2 |
| HVPGGGSVQIVYKPVDLSK (SEQ ID NO: 24) | 660.7025 | 3 | 347.2294 | 1 | y3 |
| HVPGGGSVQIVYKPVDLSK (SEQ ID NO: 24) | 660.7025 | 3 | 658.3776 | 1 | y6 |
| HVPGGGSVQIVYKPVDLSK (SEQ ID NO: 24) | 660.7025 | 3 | 795.4491 | 2 | y15 |
| IGSLDNITHVPGGGNK (SEQ ID NO: 187) | 526.9465 | 3 | 733.3739 | 2 | y15 |
| IGSLDNITHVPGGGNK (SEQ ID NO: 187) | 526.9465 | 3 | 704.8631 | 2 | y14 |
| IGSLDNITHVPGGGNK (SEQ ID NO: 187) | 526.9465 | 3 | 529.2734 | 1 | y6 |
| IGSLDNITHVPGGGNK (SEQ ID NO: 187) | 526.9465 | 3 | 604.8051 | 2 | y12 |
| IGSLDNITHVPGGGNK (SEQ ID NO: 187) | 526.9465 | 3 | 661.3471 | 2 | y13 |

In the sixth and seventh aspects of this invention, the kit may include materials for use in mass spectrometry analysis of protein samples such as buffers, diluents and/or other media, optionally further comprising instructions for use of the kit in a method of the invention. The sample collection device may comprise additional preservatives and/or stabilisers.

The kits of the invention may also be used to assess the efficacy of new therapeutics or vaccines in clinical or pre-clinical studies for the treatment of a neurodegenerative disease. Reference peptides, which may be synthetic peptides or proteolytic peptides derived from a recombinant, heavy isotope-labelled protein, may be used to pre-configure the mass spectrometer prior to use in a method of the invention to detect and quantitate the concentration of peptides of Tables 1, 2 or 3 in a sample. The reference peptides also allow for the construction of calibration lines with each batch of samples tested in order to ensure robust results. Heavy isotope-labelled peptides may be used as internal standards to control analytical variability in each sample and also provide for calibration lines. Exemplary reference peptides are listed in Table 8.

In the sixth and seventh aspects of this invention, the kit may include also include reference peptides or polypeptides from generic proteins such as Human Serum Albumin (HSA) as positive controls or for quality control, e.g. when samples have been depleted or enriched, the HSA control can give an indication of carry-through of unwanted proteins. Similarly the presence of Haemoglobin may indicate whether red blood cells have ruptured before being removed which may add extraneous material to the sample. Table 5 lists some QC proteins and peptides that might be included in a kit according to the sixth and seventh aspects of the invention.

TABLE 5
Additional proteins and peptides that are useful controls

| UniProt | Biomarker | Biomarker | Peptide | | Standard |
|---|---|---|---|---|---|
| ID | Name | Abbreviation | Mass | Sequence | Type |
| P02769 | Bovine Serum | BSA | 1398.674 | TVMENFVAFVDK | Native |
| | Albumin | | | (SEQ ID NO: 37) | |
| P02768 | Human Serum | HSA | 1012.5917 | LVAASQAALGL | Native |
| | | | | (SEQ ID NO: 38) | |
| P02768 | Albumin | HSA | 1341.6275 | AVMDDFAAFVEK | Native |
| | | | | (SEQ ID NO: 39) | |
| P68871 | Haemoglobin | HBB | 1313.6575 | VNVDEVGGEALGR | Native |
| | Subunit Beta | | | (SEQ ID NO: 40) | |

In the sixth and seventh aspects this invention, the kit can include any of the transition ions described in Table 4. These transition ions are produced when peptides derived from the proteins in Table 1 are undergoing analysis by mass spectrometry according to the methods of this invention. Preferably, the kit includes any of the transition ions described in Table 4. Alternatively, the kit may include any 2 or 3 transition ions for each peptide described in Table 4 or the kit may include any 3, 4 or 5 transition ions for each peptide disclosed in Table 2.

In the sixth and seventh aspects of the invention, each of the transition ions in the kit corresponds to and/or uniquely identifies a different peptide. Alternatively, 90% of the transition ions in the kit correspond with and/or are derived from a peptide that no other transition ion in the kit corresponds. Alternatively, 80, 70, 60, 50, 40, 30, 20, 10 or 0% of the transition ions in the kit correspond to and/or uniquely identify a peptide that no other transition ion in the kit corresponds.

In the sixth and seventh aspects of the invention, the kit may comprise synthetic peptides selected from any of the peptides described in Tables 1, 2 or 3. Preferably, the kit includes any single peptide described in Table 1 and Table 2. Alternatively, the kit includes, any 2, 3, 4, 5, 6, 7, 8, 9, 10, 11, 12, 13, 14, 15, 16, 17, 18, 19, 20, 21, 22, 23 or all 24 of the peptides described in Tables 1 and 2. Alternatively, the kit may include any single peptide described in Table 2 and Table 3. Alternatively, the kit includes, any 2, 3, 4, 5, 6, 7, 8, 9, 10, 11, 12, 13, 14, 15, 16, 17, 18, 19, 20 or all 21 of the peptides described in Tables 2 and 3. Alternatively, the kit may include any single peptide described in Table 1, Table 2 and Table 3. Alternatively, the kit includes, any 2, 3, 4, 5, 6, 7, 8, 9, 10, 11, 12, 13, 14, 15, 16, 17, 18, 19, 20, 21, 22, 23 24, 25, 26, 27, 28, 29, 30, 31, 32, 33, 34, 35 or all 36 of the peptides described in Tables 1, 2 and 3. The synthetic peptides may be modified with heavy isotopes.

In the sixth and seventh aspects of the invention, the kit may include recombinant polypeptides comprising one or more of the peptides described in Tables 1, 2 or 3. Preferably the kit includes one or more polypeptides comprising peptides described in Tables 1, 2 or 3. Alternatively, the kit includes any 3, 4, 5, 6, 7, 8, 9 polypeptides comprising peptides described in Tables 1, 2 or 3. Preferably, the recombinant polypeptides are modified with heavy isotopes. Exemplary polypeptide standards are disclosed in Table 7.

In the sixth and seventh aspects of the invention, the kit may include a mixture of peptide and polypeptide standards.

All the features of the first to fifth aspects of the present invention apply mutatis mutandis to the sixth aspect of the present invention and vice versa.

All the features of the first to sixth aspects of the present invention apply mutatis mutandis to the seventh aspect of the present invention and vice versa.

### Definitions

As used herein, “m/z” indicates the mass-to-charge ratio of an ion.

As used herein, “collisionally induced dissociation” or “CID” refers to the process by which a peptide ion is fragmented in the collision cell of a mass spectrometer.

As used herein, “CE” refers to “collision energy”, the amount of energy precursor ions receive as they are accelerated into the collision cell.

As used herein, “transition” refers to a pair of m/z values associated with a peptide, the m/z of the intact peptide ion and the m/z of a fragment ion generated by CID. Normally, heavy isotope labeled synthetic peptides are used as internal controls in SRM assays. Correlation may be used to confirm the identity of protein transitions with high confidence. The correlation between a pair of transitions is obtained from their expression profile over all samples in the training set study detailed below. As expected, transitions from the same peptide are highly correlated. Similarly, transitions from different peptide fragments of the same protein are also highly correlated. In contrast, transitions form different proteins are not highly correlated. This methodology enables a statistical analysis of the quality of the protein's SRM assay. For example, if the correlation of the transitions from the two peptides from the same protein is above 0.5 then there is less than a 5% probability that the assay is false.

As used herein, “Protein Affinity Ligand” refers to any suitable ligand with a high affinity for a target protein of interest. As used herein, “Peptide Affinity Ligand” refers to any suitable ligand with a high affinity for a target peptide of interest. Protein Affinity Ligands or Peptide Affinity Ligands may be antibodies but nucleic acid aptamers, peptide ligands, small-molecule ligands or antibody-like polypeptides such as affibodies are all suitable and one of ordinary skill in the art will be familiar with the many options available most of which can be used interchangeably.

As used herein, “IP” refers to “Immuno-Precipitation” and may be used interchangeably with the term “Immunocapture”, which in turn refers to a range of methods to isolate proteins of interest from biological samples. Typically, antibodies are used and most commonly Immunoglobulin G antibodies are used hence the term ‘Immuno’ being used to describe the capture process but IP may also employ Protein Affinity Ligands that are not antibodies.

As used herein, a “tryptic peptide” refers to the peptide that is formed by the treatment of a protein with trypsin.

As used herein, a proteotypic peptide refers to a peptide that has been to be unique to the protein of interest, typically having a unique amino acid sequence that is not present in any other protein.

As used herein, “LC” refers to “liquid chromatography”. Typically, one or more LC purification steps are performed prior to a subsequent MS/MS analysis step. Traditional LC analysis relies on the chemical interactions between sample components and column packing materials, where laminar flow of the sample through the column is the basis for separation of the analyte of interest from the test sample.

As used herein, “RT” refers to “retention time”, which is the elapsed time between injection and elution of an analyte in a chromatographic separation. Peptides are preferably selected to have differing retention times in a particular chromatographic separation to maximise sensitivity as the mass spectrometer can spend more time detecting one peptide if it elutes separately from other analyte peptides, referred to as increasing the dwell time of the mass spectrometer. Selecting peptide panels with well separated retention times is critical to the success of designing peptide panels for multiplexed assays to maximise the number of peptides that can be quantified with the highest sensitivity.

As used herein “DP” refers to the “declustering potential”, a voltage potential to dissolvate and dissociate ion clusters. It is also known as “fragmentor voltage” or “ion transfer capillary offset voltage” or “cone voltage” depending upon the mass spectrometer manufacturer.

As used herein, “MS/MS” represents tandem mass spectrometry, which is a type of mass spectrometry involving multiple stages of mass analysis with some form of fragmentation occurring in between the stages.

As used herein, “SRM” is an acronym for “selected reaction monitoring” and may be used interchangeably with “MRM”, which is an acronym for “multiple reaction monitoring”. Both SRM and MRM are targeted MS/MS methods in which molecular analytes (which for the purposes of this invention are peptides), are identified by isolating peptide precursor ions with a pre-determined m/z, subjecting the peptide ion to CID and then isolating one or more product ions with pre-determined m/z values for detection. The combination of precursor ion m/z and product ion m/z is referred to as a transition as defined above.

As used herein, “PRM” is an acronym for “parallel reaction monitoring” and is a method that is very similar to SRM and MRM but where many or all of the fragment ions of a particular peptide are acquired simultaneously, i.e. one or more single transitions are replaced with isolation of a single peptide precursor ion and detection of most or substantially all of the fragment or product ions.

As used herein, “reference peptides” are peptides that have a substantially identical sequence when compared to a natural peptide derived from enzymatic cleavage of a protein (such as a target peptide described herein) but the reference peptides are resolvable from the natural peptides and are independently quantifiable, typically through incorporation of heavy isotopes. Preferably, the abundance of the reference peptides is known and the results are compared to LC-MS/MS results from a natural peptide found in a sample to the LC-MS/MS results for the corresponding reference peptide. This allows for the calculation of the abundance of the peptide in the sample. By knowing the abundance of a peptide in a sample and the abundance of the corresponding reference peptide, the abundance of the protein it corresponds to may be determined. The term “precursor reference peptides” refers to reference peptides that comprise additional flanking sequences at one or both ends of the peptide that need to be digested from the precursor reference peptide by a protease to release the actual reference peptide. Likewise a “reference polypeptide” refers to a protein that contains the reference peptide sequence and again must be digested with a protease to release the reference peptide. A reference polypeptide may be a recombinant protein and may also be modified with heavy isotopes. A reference peptide or precursor reference polypeptide can comprise an amino acid sequence selected from the target peptides described herein, e.g. in tables 1 to 3, or a variant thereof. A suitable variant comprises at least one amino acid comprising heavy isotopes such as ²H, ¹³C ¹⁵N or ¹⁸O.

As used herein, “synthetic peptides” are peptides that are functionally identical to natural peptides derived from enzymatic cleavage of proteins but synthetic peptides are produced by chemical synthesis e.g. automated solid phase peptide synthesis. Synthetic peptides can be used as controls for the abundance of the proteins they are derived from and/or correspond to. Preferably, the abundance of the synthetic peptides is defined and the results are compared to LC-MS/MS results from a natural peptide found in a sample to the LC-MS/MS results for the corresponding synthetic peptide. This allows for the calculation of the abundance of the peptide in the sample. By knowing the abundance of a peptide in a sample and the abundance of the corresponding reference peptide, the abundance of the protein it corresponds to may be determined.

As used herein, “recombinant peptides” or “recombinant polypeptides” refer to peptides or proteins that are produced by expression of an artificial DNA vector such as a plasmid in E. coli. Recombinant peptides or polypeptides can be generated using any method known in the art. These methods can include recombinant expression techniques such as expression in bacteria or in vitro expression in eukaryotic cell lysate.

As used herein, “SIIS” refers to “Stable Isotope Internal Standard” which are generally heavy-isotope modified synthetic peptides or heavy-isotope modified recombinant polypeptides. SIIS materials can be isotopically labeled. The isotopes with which they are typically labelled include ¹³C, ²H, ¹⁵N and ¹⁸O. The SIIS materials can also include a polar solvent. Polar solvents can include acetonitrile, water, mixtures of acetonitrile and water and mixtures of ethanol and water. In some circumstances, particularly hydrophobic peptides may be solubilized in Dimethylsulphoxide (DMSO).

As used herein, a “biological sample” refers a sample of biological material that is to be analysed using the methods of this invention. In certain applications of the invention, the samples described herein are taken from mammals. These mammals include rats, mice, rabbits, dogs, non-human primates and humans. Samples can be isolated from any tissue or organ or from any bodily fluid. Organs from which samples can be taken include skin, heart, lung, brain, kidney, liver, pancreas, spleen, testes, ovaries, gall bladder, thymus, thyroid, eye, ear, nose, mouth, tongue, penis, vagina, bladder or larynx. Tissues include nervous tissue, vascular tissue, muscle, bone, gastrointestinal tract, epithelial tissue, fibroblastic tissue, mucous membranes, hair, skin, reproductive tissue and connective tissue. Body fluids and excretions include, blood, serum, saliva, urine, semen, vaginal secretions, stool, bile, tears, lymph, ear wax, mucous, shed skin, finger nails, toe nails, skin oils, sweat and dandruff.

As used herein, “machine-readable media” refers to any practical means by which data can be read by a computer, including optical media such compact disks (CDs) or digital versatile disks (DVDs), flash drives, hard drives or even digital files downloaded from the internet.

A “proteolytic peptide” is a peptide sequence from a protein, which has been produced by the action of a protease cleaving a peptide bond between amino acids in the protein sequence. Such proteolytic peptides are therefore oligopeptides being formed of a number of amino acids. The proteolytic peptide may be from 5 to 30 amino acid residues in length, suitably 6 to 20 amino acids in length, 7 to 25 amino acids in length, or any of 5, 6, 7, 8, 9, 10, 11, 12, 13, 14, 15, 16, 17, 18, 19, 20, 21, 22, 23, 24, 25, 26, 27, 28, 29 or 30 amino acids in length.

As used herein, the term “Subtype” refers to the variants of Microtubule Associated Tau Protein that are produced by alternative splicing giving rise to proteins that differ in parts of their sequence from other Subtypes of Microtubule Associated Protein Tau. This term is largely interchangeable with the term “Isoform” although isoform refers specifically to particular isoforms while subtypes, in the context of this invention refers primarily to which tissue is producing the variants of Tau and subtypes of MAPT produced by the Central Nervous System encompasses multiple isoforms of MAPT that do not comprise Exon 4a while subtypes of MAPT produced by the Peripheral Nervous System encompass multiple isoforms of MAPT that do comprise Exon 4a.

As used herein, the term “Total Tau” refers to the Total amount of Microtubule Associated Tau Protein in blood or blood derived fluids such as plasma, serum, sweat, saliva and urine, irrespective of the tissue that or organ system that produces the Tau Protein. For the purposes of this invention, it is preferred that the absolute quantity of Total Tau is determined by measurement of the absolute quantity of proteolytic peptides, such as tryptic peptides, that are common to all subtypes or isoforms of Tau. The peptides labelled ‘Total Tau’ in tables 1, 2, and 3 are proteolytic peptides that are common to all variants of the Tau protein.

As used herein, the term “PNS Tau” or “Peripheral Nervous System Tau” refers to the amount of Microtubule Associated Tau Protein in blood or blood derived fluids such as plasma, serum, sweat, saliva and urine, that is produced by cells of the Peripheral Nervous System. For the purposes of this invention, it is preferred that the absolute quantity of PNS Tau is determined by measurement of the absolute quantity of proteolytic peptides, such as tryptic peptides, that are unique to subtypes of Tau expressed by the Peripheral Nervous System. In particular, PNS Tau is known to comprise an additional segment of polypeptide that is the result of splicing. subtypes of PNS Tau typically comprise a stretch of polypeptide sequence encoded by exon 4a of the MAPT gene. Thus the absolute quantity of subtypes of Microtubule Associated Protein Tau Expressed by the Peripheral Nervous System can be determined from one or more peptides that comprise one or more amino acids encoded by exon 4a of the MAPT gene. Suitable peptides include those peptides shown in Tables 1, 2 and 3 marked as ‘PNS-Tau’ peptides. PNS Tau may also be referred to interchangeably as “High Molecular Weight Tau”, “HMW Tau” or “Big Tau”.

As used herein, the term “CNS Tau” or “Central Nervous System Tau” refers to the amount of Microtubule Associated Tau Protein in blood or blood derived fluids such as plasma, serum, sweat, saliva and urine, that is produced by cells of the Central Nervous System. For the purposes of this invention, it is preferred that the absolute quantity of CNS Tau is determined by measurement of the absolute quantity of proteolytic peptides, such as tryptic peptides, that are unique to subtypes of Tau expressed by the Central Nervous System. Subtypes of CNS Tau are known to comprise peptides that span the splice boundary between exons 4 and 5 of the MAPT gene that are not present in PNS Tau due to the presence of the additional exon 4a between exons 4 and 5 in the PNS Tau sequence. Thus the absolute quantity of Subtypes of Microtubule Associated Protein Tau Expressed by the Central Nervous System can be determined from one or more peptides that span the splice boundary between exons 4 and 5 of the MAPT gene, such as one or more of those peptides shown in Tables 1, 2 and 3 marked as ‘CNS-Tau’ peptides. The group of peptides that span the splice boundary between exons 4 and 5 of the MAPT gene includes peptides comprising or consisting of the amino acid sequence DEAAGHVTQAR (SEQ ID NO: 25). Proteolytic peptides that are specific for CNS Tau can include peptides comprising or consisting of the amino acid sequence DEAAGHVTQAR (SEQ ID NO: 25) and/or peptides comprising the sequence VQIVYKPV (SEQ ID NO:26) at the N-terminus of the peptide. The absolute quantity of CNS Tau may also be determined by subtracting the absolute quantity of PNS Tau from the absolute quantity of Total Tau. CNS Tau may also be referred to interchangeably as “Low Molecular Weight Tau” or “LMW Tau”.

Diagnostically useful measurements of Tau include the Ratio of CNS Tau to PNS Tau, the ratio of CNS Tau to Total and the ratio of PNS Tau to Total Tau may be diagnostically relevant to determine. In the methods described herein the severity of the neurodegenerative disease of the subject can be diagnosed and/or classified according to one or more of the diagnostically useful measurements provided, as well as or instead of according to the relative concentration of at least one target proteolytic peptide.

As used herein, the “Velocity of CNS Tau” refers to the measurement of changes in the absolute quantities of CNS Tau in patients at different times-points and determining the differences in the amounts of CNS Tau at these different time-points to determine whether the amounts of CNS Tau are increasing or decreasing. This may be useful in determining whether a drug is effective in the treatment of a Neurodegenerative Disease such as Alzheimer's disease. For example, this would entail measuring CNS Tau at a first time, e.g. before treatment with a drug and the measuring CNS Tau at a second time, e.g. after treatment with a drug. The two values maybe subtracted from each other to determine whether there is a meaningful change occurring. Preferably, the rate of change of CNS Tau over time is determined e.g. by determining amounts of CNS Tau in blood or blood-derived body fluids at multiple time-points and optionally plotting on a graph to show the rate of change of CNS Tau over time. Velocity of CNS Tau may also be used to determine severity and rate of progression of a Neurodegenerative Disease.

For the avoidance of doubt, it is hereby expressly stated that features described herein as ‘preferred’, ‘preferable’, ‘desirable’, ‘advantageous’, ‘convenient’ or the like may be present in the invention in isolation or in any combination with any one or more other features, so described (unless the context dictates otherwise) and this constitutes and explicit disclosure of such combinations of features.

### DETAILED DESCRIPTION OF THE INVENTION

The object of the present disclosure is to provide improved methods for the use of LC-MS/MS SRM in assays for neurodegenerative disease. More specifically, in a first aspect of the invention, a method is provided for quantifying by mass spectrometry the amount of a subtype of the Microtubule Associate Protein Tau that is in a biological sample from a subject by measuring the absolute quantity of at least one target peptide that is substantially unique to the subtype being quantified with reference to at least one corresponding reference peptide(s), in a method comprising the steps:
(i) either, adding at least one reference peptide or at least one precursor reference peptide(s) or at least one reference polypeptide to the biological sample followed by incubating the biological sample with at least one protease to form a proteolytic digest of Tau proteins in the sample so that the protease releases the at least one target peptide from the Tau subtypes present and also releases the reference peptide from the precursor reference peptide or reference polypeptide; followed by isolating the Tau peptides and reference peptides from the proteolytic digest of the sample to form a target proteolytic digest;
   or, incubating the biological sample with at least one protease to form a proteolytic digest of Tau proteins in the sample so that the protease releases the at least one target peptide from the Tau subtypes present; followed by adding the at least one reference peptide(s) to the proteolytic digest and then isolating the Tau peptides from the proteolytic digest of the sample to form a target proteolytic digest;
(ii) assaying the target proteolytic digest for the presence of the at least one target proteolytic peptide that is substantially unique to the isoform of the Microtubule Associate Protein Tau and the at least one corresponding reference peptide(s), using mass spectrometry; optionally wherein the subtype is the Microtubule Associate Protein Tau expressed by the Central Nervous System or the Microtubule Associate Protein Tau expressed by the Peripheral nervous system.

Other aspects of the invention provide methods for diagnosis and classification of the severity of a neurodegenerative disease, methods for assessing the effectiveness of treatments for a neurodegenerative disease and kits for carrying out the methods of this invention.

### Biological Samples:

In most aspects of the invention a biological sample is acquired from a subject. The biological sample is not particularly limited but preferably should contain at least one of the proteins of interest unless the sample is being used as a negative control in which case the target proteins should not be present. For the purposes of assaying proteins related to Neurodegenerative disease, the biological sample is preferably a blood sample, which may be whole but is preferably processed to extract plasma or serum. Cerebrospinal fluid (CSF) is an alternative preferred biological sample. Likewise, tissue biopsies such as brain and spinal cord material are also preferred samples although these are more typically obtained post-mortem while blood and CSF can be obtained from live patients and can be sampled repeatedly. Alternatively, other body fluids such as saliva, urine and sweat may also be possible sources of possible biological samples. Cell lines and animal model tissue or blood may also be suitable biological samples for use in this invention.

In some applications of the invention, the biological disease and biological control samples may be processed using an immune-depletion method prior as part of the step of isolating a protein sample from the disease or control samples prior to LC-SRM-MS analysis. Immuno-depletion involves removal of one or more proteins through the use of antibodies. Numerous immune-depletion techniques are known to those of skill in the art. Immuno-depletion is typically used with samples like plasma, CSF or urine where there may be a high background of proteins that are uninformative for the purposes of the assay such as albumin or immunoglobulins and where it is preferable to remove those proteins.

### Immuno-Precipitation:

In the first aspect of the invention, the biological samples may be processed using an immuno-capture method or immuno-precipitation prior to LC-MS/MS analysis. Immuno-precipitation (IP) involves isolation of one or more proteins through the use of antibodies or other protein or peptide affinity ligands. Numerous immuno-capture techniques are known to those of skill in the art. In general, however, preferred methods employ protein capture ligands such as antibodies (Gao et al., J Immunoassay Immunochem, 39(4):351-364, “A brief review of monoclonal antibody technology and its representative applications in immunoassays”, 2018) or protein affinity ligands such as affibodies (Stahl et al., Trends Biotechnol 35(8):691-712, “Affibody Molecules in Biotechnological and Medical Applications”, 2017) or aptamers (Zhang et al, Molecules 24(5):941-963, “Recent Advances in Aptamer Discovery and Applications”, 2019) that are immobilised on a solid phase support. Preferably, the antibodies or other protein affinity ligands are immobilised on magnetic microparticles. A generic approach to create antibody-coated magnetic particles is to use commercially available magnetic particles such as Dynabeads™ that are pre-coated with covalently immobilised Protein G (ThermoFisher Scientific, Cat No: 10009D). Protein G will bind to immunoglobulin G (IgG) antibodies and there are numerous suppliers of IgG antibodies targeting a substantial number of proteins. Another favoured generic approach to generate particles suitable for IP assays is to use epoxy-derivativised magnetic particles such as M270 epoxy Dynabeads™ (ThermoFisher Scientific, Cat No: 10009D). This approach can be used generically with any protein affinity ligand that comprises a free amino or free thiol group to react with the epoxy functionality unlike Protein G Dynabeads, which will only work with IgG antibodies. In another preferred approach is to use M-280 Streptavidin-derivatised Dynabeads™ to capture biotin-derivatised antibodies or other biotin derivatised protein affinity ligands. Again, the use of streptavidin/biotin interactions is a highly flexible and generic way to create solid supports linked to Protein Affinity Ligands for the purposes of Immuno-Precipitation. Magnetic particles are advantageous as they can be conditionally separated from the supernatant in the presence of a magnet allowing the supernatant to be washed or otherwise separated from the solid phase support.

Other preferred supports include silica particles derivatised with epoxy functionalities, Protein G, Protein A, Avidin or Streptavidin. Other solid supports are also possible using thiol, amine or carboxyl derivatised supports which can all provide handles for covalently linking Protein Affinity ligands to a solid support. Non-magnetic supports are also preferred but they need to centrifuged to separate the particles from supernatant rather than having the particles aggregated by a magnet.

### Immuno-Precipitation of Whole Proteins:

In applications of this invention where target proteins are isolated by immuno-precipitation prior to digestion, the antibody that is selected to isolate the target protein can bind to any epitope in the target protein giving a wider range of antibodies to choose from and there is a large number of suppliers of antibodies for the protein targets of this invention. In general, any antibody or other type of protein affinity ligand that has a reasonable binding affinity for the target proteins of interest can be used with this invention. However, in some embodiments the proteolytic peptides are isolated after digestion of the patient sample. In these embodiments the antibodies selected for isolation of the target peptides must bind to the target peptides specifically separate from the context of the intact protein and some antibodies that bind well to the intact protein may not bind well to peptides derived from the same protein. Many antibodies are generated against linear peptide epitopes and so it is often possible to identify suitable antibodies if the manufacturer discloses the epitope sequence that was used to generate the antibody against a given target protein. If the epitope comprises the sequence of the target proteotypic peptide or a partial sequence then the antibody is likely to bind to the target peptide and is a peptide affinity ligand. Sometimes, antibodies are generated against the whole protein and in this instance the antibody may have an affinity for the target peptide but this will have to be determined empirically. Similarly, a polyclonal antibody may comprise antibodies against a range of peptide epitopes in a target protein and so many polyclonal antibodies may be suitable to pull down linear proteolytic peptide sequences on their own but again this will have to be determined empirically. Many antibody suppliers provide polyclonal antibodies, which can be investigated for suitability to isolate target peptides of this invention. Table 6 provides some exemplary antibodies act as peptide affinity ligands that have been found by the inventors to bind to target peptides of this invention.

TABLE 6
Exemplary Antibodies that bind to Specific Target
Peptides of this invention:

| | | Antibody | | |
|---|---|---|---|---|
| | | (Supplier; | | |
| | | Product | | |
| Biomarker | Sequence | No) | Epitope | Class |
| Tau | TPSLPTPPTR | Genscript; | TPSLPTPPT | Poly- |
| (Total) | (SEQ ID | Custom | R (SEQ ID | clonal |
| | NO: 41) | Antibody | NO: 41) | |
| | GAAPPGQK | Genscript; | GAAPPGQK | Poly- |
| | (SEQ ID | Custom | (SEQ ID NO: | clonal |
| | NO: 42) | Antibody | 42) | |

### Heavy Recombinant Protein Standards and Reference Peptides:

A mass spectrometric internal standard is a molecule that displays physical and chemical characteristics similar or essentially identical to that of an analyte of interest, but at the same time generates a signal that can be distinguished from that of the analyte. A predetermined amount of internal standard can added to a sample to be analyzed, and due to the similarity between the analyte and the internal standard, the ratio of the signals of the standard to the analyte will reflect the ratio of the actual quantities of the standard and the analyte thus knowing the amount of standard allows the amount of the analyte to be determined. If added early in the processing of a sample, because both standard and analyte suffer the same losses due to inefficiencies in extraction, digestion or other processing steps, accuracy of quantification is achieved. For the analysis of target proteins that have been enzymatically digested, heavy isotope doped peptides may be used as identical but mass-spectrometrically resolvable internal standards for the quantification of the target proteins.

In embodiments of this invention where the target proteins are isolated prior to digestion, it is generally preferable to use reference polypeptides that are pulled down along with the native target proteins to act as internal standards, where the reference polypeptides are typically recombinant versions of the target proteins. Heavy recombinant proteins can be created by growing the bacteria that will express the recombinant proteins in a medium comprising heavy amino acids. In these embodiments of the first aspect of the invention, the internal standard reference proteolytic peptide is derived from digestion of a recombinant heavy protein substantially comprising the sequence of the target protein that includes at a minimum the sequences of the target proteolytic peptides of the assay. Heavy recombinant protein reference polypeptides are preferred when target proteins are isolated prior to proteolytic digestion as these can be added to the sample as soon as it is obtained. The heavy reference proteins are then subjected to all the same processing steps as the native target proteins including immuno-precipitation and digestion so that the reference peptides act as a very accurate internal standard for the quantification of the native target proteins and correct for variability in quantitative measurements that may be caused by differences in sample processing in the immuno-precipitation and proteolytic digestion steps, which are the points in the method where variability might be expected to be greatest, e.g. if proteolysis of the native proteins does not proceed as completely as it could due to an error in pipetting of the protease, the heavy protein internal standard will be subject to the same level of proteolysis and so the test will still give an accurate quantitative result due to presence of the reference protein. This is a key feature of the invention.

Heavy recombinant proteins are commercially available from various specialist manufacturers including GenScript, Inc (NJ, USA), Creative Biomart Inc, (NY, USA), Promise Proteomics (Grenoble, France). Exemplary light and heavy reference polypeptides for use with this invention are listed in Table 7. Preferably, the reference polypeptides for use in the aspects of the present invention comprise one or more reference polypeptides selected from those listed in Table 7, which all correspond to UniProt ID P10636.

TABLE 7
Recombinant Reference Polypeptide Standards and Suppliers:

| | Biomarker | | | Standard |
|---|---|---|---|---|
| Biomarker Name | Abbn | Supplier | Product Number: | Type |
| Tau-441 | MAPT | SigmaAldrich | Ref # SRP0701 | Light |
| Microtubule-Associated | MAPT | Promise | TAU 441 - | Heavy |
| Protein Tau | | Proteomics | LABELLED | |
| | | | (ARG, LYS) 13C15N | |
| Microtubule-Associated | MAPT | rPeptide | Catalog ID: | Heavy |
| Protein Tau | | | T-1101-1 | |

In embodiments of this invention where the target proteins are digested prior to isolation, it is also possible to use reference polypeptides such as those listed in Table 7.

It is also possible to use synthetic reference peptides as the internal standards. Synthetic reference peptides can be cheaper to produce and may allow post-translational modifications (PTM) to be more readily quantified as long as the PTM can be produced synthetically. In these embodiments of the invention the reference proteolytic peptide is synthesized to be have the same sequence as the target proteolytic peptide expected to be present in the patient sample. The synthetic reference proteolytic peptide may also comprise additional amino acids as a digestion control (Faria et al., Chromatogr B Analyt Technol Biomed Life Sci, 1001:156-68, “Comparison of a stable isotope labeled (SIL) peptide and an extended SIL peptide as internal standards to track digestion variability of an unstable signature peptide during quantification of a cancer biomarker, human osteopontin, from plasma using capillary microflow LC-MS/MS”, 2015). Part of the native protein sequence flanking the sequence of the target peptide may be included at the ends of the synthetic reference peptide as a digestion control. These are referred to as digestion tagged peptides or precursor reference peptides. Flanking sequence may be added at one terminus of the peptide or the other or can be added at both ends of the peptide if desired. Incorporation of 3 to 5 amino acids of adjacent sequence is typical although more can be included if desired. The length of flanking sequence at either end does not need to be the same but may be identical if desired. The synthetic reference peptides or precursor reference peptides are typically also modified with heavy amino acids such as heavy Lysine, heavy Arginine, heavy Leucine, heavy Valine or heavy Proline. These amino acids are preferred as they can introduce at least 6 heavy nuclei into a peptide although other amino acids can be used if necessary. Peptides with heavy isotopes can be prepared by standard solid phase FMOC peptide synthesis using commercially available heavy amino acids such as heavy Lysine (L-LYSINE-ALPHA-N-FMOC, EPSILON-N-T-BOC (13C6, 99%; 15N2, 99%); Item No: CNLM-4754-H-PK available from Cambridge Isotope Laboratories, MA, USA), heavy Arginine (L-ARGININE-N-FMOC, PBF-OH (13C6, 99%; 15N4, 99%); Item No: CNLM-8474-H-PK available from Cambridge Isotope Laboratories, MA, USA), heavy Leucine (L-LEUCINE-N-FMOC (13C6, 99%; 15N, 99%); Item No: CNLM-4345-H-PK available from Cambridge Isotope Laboratories, MA, USA), heavy Valine (L-VALINE-N-FMOC (13C5, 99%; 15N, 99%); Item No: CNLM-4348-H-PK available from Cambridge Isotope Laboratories, MA, USA) or heavy proline (L-PROLINE-N-FMOC (13C5, 99%; 15N, 99%); Item No: CNLM-4347-H-PK available from Cambridge Isotope Laboratories, MA, USA).

The heavy reference peptides are added to the patient sample as soon as possible after being obtained from the patient so that the reference peptides are then subjected to all the same processing steps as the native target proteins including digestion and immune-precipitation; in this way the reference peptides act as a very accurate internal standard for the quantification of the native target proteins and correct for variability in quantitative measurements that may be caused by differences in sample processing in the proteolytic digestion and immuno-precipitation steps, which are the points in the method where variability might be expected to be greatest, e.g. if proteolysis of the native proteins does not proceed as completely as it could due to an error in pipetting of the protease, the heavy peptide internal standard, if it comprises a digestion tag, referred to as a precursor reference peptide, will be subject to the same level of proteolysis and so the test will still give a more accurate quantitative result due to presence of the reference peptide. Likewise, reference polypeptides need to be digested to release the reference peptide sequence and reference polypeptides will act as digestion controls. This is a key feature of the invention and in preferred embodiments of the invention precursor reference peptides or reference polypeptides are used rather than the reference peptide on their own.

One of ordinary skill in the art will be aware that reference peptides that do not comprise flanking sequences that need to be digested, can be added after digestion of the biological sample. Thus preparing the biological sample from the subject for assay by adding a predetermined amount of reference peptides, precursor reference peptides or reference polypeptides to the sample can take place at any point in the method before assaying the target proteolytic digest, such as before isolating the tau proteins from the sample, before incubating the isolated target proteins with a protease, or after isolating the target peptides from the proteolytic digest of the sample to form a target proteolytic digest. Adding the reference peptides to the sample after isolating the target peptides from the proteolytic digest of the sample is typically less preferred as the later in the process that reference peptides are added to a sample, the greater the variance there will be in the final measured quantities of the target peptides reducing the quantitative accuracy of the method.

Heavy recombinant peptides can be prepared by any commercial peptide synthesis service using the commercially available FMOC-amino acid precursors listed above. Exemplary light and heavy reference peptides for use with this invention are listed in Table 8 but one of ordinary skill in the art will be aware that many minor variations of the sequences disclosed in Table 8 are possible and will work satisfactorily, such as shorter or longer flanking sequences or different lengths of flanking sequence at either end of the reference peptide sequence. Preferably, the heavy reference polypeptides for use in the aspects of the present invention comprise one or more heavy reference polypeptides selected from those listed in Table 8.

TABLE 8
Exemplary Reference Peptide Sequences and Precursor Reference Peptide Sequences.
Letters in bold in the sequence entries are sites where a heavy amino acid is
introduced to generate the heavy reference peptide. The Heavy Peptide Mass is
calculated assuming that peptides labelled with heavy lysine are labelled with
Lys[13C6,15N2]and peptides labelled with heavy arginine are labelled with Arg
[13C6,15N4]. Underlined letters in the Digestion Tagged Precursor Reference
Peptide Sequence entries indicates the Digested Peptide sequence that is
expected to be cleaved from the Digestion Tagged Peptide during proteolytic
digestion.

| | | Native | Heavy | |
|---|---|---|---|---|
| | | Peptide | Peptide | Digestion Tagged Reference Peptide |
| Biomarker | Reference Peptide Sequence | Mass | Mass | Sequence (Precursor Reference Peptides) |
| CNS Tau | AEEAGIGDTPSLEDEAAGHVTQAR | 2423.1200 | 2433.1283 | DAGLK-AEEAGIGDTPSLEDEAAGHVTQAR (SEQ ID NO: 79) |
| | (SEQ ID NO: 43) | | | DAGLK-AEEAGIGDTPSLEDEAAGHVTQAR-MVSKS (SEQ ID NO: 80) |
| | | | | AEEAGIGDTPSLEDEAAGHVTQAR-MVSKS (SEQ ID NO: 81) |
| | QAAAQPHTEIPEGTTAEEAGIGDTPSLEDEAAG | 3954.8467 | 3964.8549 | GAPGK-QAAAQPHTEIPEGTTAEEAGIGDTPSLEDEAAGHVTQAR (SEQ ID |
| | HVTQAR (SEQ ID NO: 44) | | | NO: 82) |
| | | | | GAPGK-QAAAQPHTEIPEGTTAEEAGIGDTPSLEDEAAGHVTQAR-MVSKS |
| | | | | (SEQ ID NO: 83) |
| | | | | QAAAQPHTEIPEGTTAEEAGIGDTPSLEDEAAGHVTQAR-MVSKS |
| | | | | (SEQ ID NO: 84) |
| | STPTAEAEEAGIGDTPSLEDEAAGHVTQAR | 3009.3799 | 3019.3881 | TSDAK-STPTAEAEEAGIGDTPSLEDEAAGHVTQAR (SEQ ID NO: 85) |
| | (SEQ ID NO: 45) | | | TSDAK-STPTAEAEEAGIGDTPSLEDEAAGHVTQAR-MVSKS(SEQ ID NO: |
| | | | | 86) |
| | | | | STPTAEAEEAGIGDTPSLEDEAAGHVTQAR-MVSKS(SEQ ID NO: 87) |
| | VQIVYKPVDLSK (SEQ ID NO: 46) | 1387.8075 | 1395.8217 | PGGGK-VQIVYKPVDLSK (SEQ ID NO: 88) |
| | | | | PGGGK-VQIVYKPVDLSK-VTSKC (SEQ ID NO: 89) |
| | | | | VQIVYKPVDLSK-VTSKC (SEQ ID NO: 90) |
| | EAGIGDTPSLEDEAAGHVTQAR (SEQ ID NO: | 2223.0403 | 2233.0486 | QQRKR-EAGIGDTPSLEDEAAGHVTQAR (SEQ ID NO: 91) |
| | 47) | | | QQRKR-EAGIGDTPSLEDEAAGHVTQAR-MVSKS(SEQ ID NO: 92) |
| | | | | EAGIGDTPSLEDEAAGHVTQAR-MVSKS(SEQ ID NO: 93) |
| | DEAAGHVTQAR (SEQ ID NO: 48) | 1153.5476 | 1163.5558 | TPSLE-DEAAGHVTQAR (SEQ ID NO: 94) |
| | | | | TPSLE-DEAAGHVTQAR-MVSKS(SEQ ID NO: 95) |
| | | | | DEAAGHVTQAR-MVSKS(SEQ ID NO: 96) |
| | VQIVYKPV (SEQ ID NO: 49) | 944.5695 | 950.5833 | PGGGK-VQIVYKPV (SEQ ID NO: 97) |
| | | | | PGGGK-VQIVYKPV-DLSKV (SEQ ID NO: 98) |
| | | | | VQIVYKPV-DLSKV (SEQ ID NO: 99) |
| PNS Tau | QPSGTGPEDTEGGR (SEQ ID NO: 50) | 1386.6011 | 1396.6094 | REATR-QPSGTGPEDTEGGR (SEQ ID NO: 100) |
| | | | | REATR-QPSGTGPEDTEGGR-HAPEL (SEQ ID NO: 101) |
| | | | | QPSGTGPEDTEGGR-HAPEL (SEQ ID NO: 102) |
| | DVDESSPQDSPPSK (SEQ ID NO: 51) | 1486.6423 | 1494.6565 | VDEDR-DVDESSPQDSPPSK (SEQ ID NO: 103) |
| | | | | VDEDR-DVDESSPQDSPPSK-ASPAQ (SEQ ID NO: 104) |
| | | | | DVDESSPQDSPPSK-ASPAQ (SEQ ID NO: 105) |
| | ASPAQDGRPPQTAAR (SEQ ID NO: 52) | 1521.7648 | 1531.7730 | SPPSK-ASPAQDGRPPQTAAR (SEQ ID NO: 106) |
| | | | | SPPSK-ASPAQDGRPPQTAAR-EATSI (SEQ ID NO: 107) |
| | | | | ASPAQDGRPPQTAAR-EATSI(SEQ ID NO: 108) |
| | EATSIPGFPAEGAIPLPVDFLSK (SEQ ID NO: 53) | 2355.2361 | 2363.2504 | QTAAR-EATSIPGFPAEGAIPLPVDFLSK (SEQ ID NO: 109) |
| | | | | QTAAR-EATSIPGFPAEGAIPLPVDFLSK-VSTEI (SEQ ID NO: 110) |
| | | | | EATSIPGFPAEGAIPLPVDFLSK-VSTEI (SEQ ID NO: 111) |
| | VSTEIPASEPDGPSVGR (SEQ ID NO: 54) | 1696.8268 | 1706.8350 | DFLSK-VSTEIPASEPDGPSVGR (SEQ ID NO: 112) |
| | | | | DFLSK-VSTEIPASEPDGPSVGR-AKGQD (SEQ ID NO: 113) |
| | | | | VSTEIPASEPDGPSVGR-AKGQD (SEQ ID NO: 114) |
| | GQDAPLEFTFHVEITPNVQK (SEQ ID NO: 55) | 2269.1379 | 2277.1521 | VGRAK-GQDAPLEFTFHVEITPNVQK (SEQ ID NO: 115) |
| | | | | VGRAK-GQDAPLEFTFHVEITPNVQK-EQAHS (SEQ ID NO: 116) |
| | | | | GQDAPLEFTFHVEITPNVQK-EQAHS (SEQ ID NO: 117) |
| | GPSLGEDTK (SEQ ID NO: 56) | 902.4345 | 910.4487 | GPEAR-GPSLGEDTK (SEQ ID NO: 118) |
| | | | | GPEAR-GPSLGEDTK-EADLP (SEQ ID NO: 119) |
| | | | | GPSLGEDTK-EADLP (SEQ ID NO: 120) |
| | EADLPEPSEK (SEQ ID NO: 57) | 1113.5190 | 1121.5332 | GEDTK-EADLPEPSEK (SEQ ID NO: 121) |
| | | | | GEDTK-EADLPEPSEK-QPAAA (SEQ ID NO: 122) |
| | | | | EADLPEPSEK-QPAAA (SEQ ID NO: 123) |
| | VVQEGFLR (SEQ ID NO: 58) | 946.5236 | 956.5319 | PESGK-VVQEGFLR (SEQ ID NO: 124) |
| | | | | PESGK-VVQEGFLR-EPGPP (SEQ ID NO: 125) |
| | | | | VVQEGFLR-EPGPP (SEQ ID NO: 126) |
| | HAPELLK (SEQ ID NO: 59) | 806.4650 | 814.4792 | TEGGR-HAPELLK (SEQ ID NO: 127) |
| | | | | TEGGR-HAPELLK-HQLLG (SEQ ID NO: 128) |
| | | | | HAPELLK-HQLLG (SEQ ID NO: 129) |
| | HQLLGDLHQEGPPLK (SEQ ID NO: 60) | 1680.8947 | 1688.9089 | PELLK-HQLLGDLHQEGPPLK (SEQ ID NO: 130) |
| | | | | PELLK-HQLLGDLHQEGPPLK-GAGGK (SEQ ID NO: 131) |
| | | | | HQLLGDLHQEGPPLK-GAGGK (SEQ ID NO: 132) |
| | EQAHSEEHLGR (SEQ ID NO: 61) | 1291.5905 | 1301.5988 | PNVQK-EQAHSEEHLGR (SEQ ID NO: 133) |
| | | | | PNVQK-EQAHSEEHLGR-AAFPG (SEQ ID NO: 134) |
| | | | | EQAHSEEHLGR-AAFPG (SEQ ID NO: 135) |
| | AAFPGAPGEGPEAR (SEQ ID NO: 62) | 1325.6364 | 1335.6446 | EHLGR-AAFPGAPGEGPEAR (SEQ ID NO: 136) |
| | | | | EHLGR-AAFPGAPGEGPEAR-GPSLG (SEQ ID NO: 137) |
| | | | | AAFPGAPGEGPEAR-GPSLG (SEQ ID NO: 138) |
| | DEAAGHVTQEPESGK (SEQ ID NO: 63) | 1553.6957 | 1561.7100 | TPSLE-DEAAGHVTQEPESGK(SEQ ID NO: 139) |
| | | | | TPSLE-DEAAGHVTQEPESGK-VVQEG (SEQ ID NO: 140) |
| | | | | DEAAGHVTQEPESGK-VVQEG (SEQ ID NO: 141) |
| | DTEGGR (SEQ ID NO: 64) | 633.2718 | 643.2801 | GTGPE-DTEGGR (SEQ ID NO: 142) |
| | | | | GTGPE-DTEGGR-HAPEL (SEQ ID NO: 143) |
| | | | | DTEGGR-HAPEL (SEQ ID NO: 144) |
| | DLHQEGPPLK (SEQ ID NO: 65) | 1132.5876 | 1140.6019 | HQLLG-DLHQEGPPLK (SEQ ID NO: 145) |
| | | | | HQLLG-DLHQEGPPLK-GAGGK (SEQ ID NO: 146) |
| | | | | DLHQEGPPLK-GAGGK (SEQ ID NO: 147) |
| | DGRPPQTAAR (SEQ ID NO: 66) | 1067.5472 | 1077.5555 | ASPAQ-DGRPPQTAAR (SEQ ID NO: 148) |
| | | | | ASPAQ-DGRPPQTAAR-EATSI(SEQ ID NO: 149) |
| | | | | DGRPPQTAAR-EATSI(SEQ ID NO: 150) |
| | DGPSVGR (SEQ ID NO: 67) | 686.3347 | 696.3430 | PASEP-DGPSVGR (SEQ ID NO: 151) |
| | | | | PASEP-DGPSVGR-AKGQD (SEQ ID NO: 152) |
| | | | | DGPSVGR-AKGQD (SEQ ID NO: 153) |
| | DAPLEFTFHVEITPNVQK (SEQ ID NO: 68) | 2084.0578 | 2092.0720 | RAKGQ-DAPLEFTFHVEITPNVQK (SEQ ID NO: 154) |
| | | | | RAKGQ-DAPLEFTFHVEITPNVQK-EQAHS (SEQ ID NO: 155) |
| | | | | DAPLEFTFHVEITPNVQK-EQAHS (SEQ ID NO: 156) |
| | DLPEPSEK (SEQ ID NO: 69) | 913.4393 | 921.4535 | DTKEA-DLPEPSEK (SEQ ID NO: 157) |
| | | | | DTKEA-DLPEPSEK-QPAAA (SEQ ID NO: 158) |
| | | | | DLPEPSEK-QPAAA (SEQ ID NO: 159) |
| Total Tau | LQTAPVPMPDLK (SEQ ID NO: 70) | 1308.7111 | 1316.7253 | SAKSR-LQTAPVPMPDLK (SEQ ID NO: 160) |
| | | | | SAKSR-LQTAPVPMPDLK-NVKSK (SEQ ID NO: 161) |
| | | | | LQTAPVPMPDLK-NVKSK (SEQ ID NO: 162) |
| | TDHGAEIVYK (SEQ ID NO: 71) | 1131.5560 | 1139.5702 | NAKAK-TDHGAEIVYK (SEQ ID NO: 163) |
| | | | | NAKAK-TDHGAEIVYK-SPVVS (SEQ ID NO: 164) |
| | | | | TDHGAEIVYK-SPVVS (SEQ ID NO: 165) |
| | GAAPPGQK (SEQ ID NO: 72) | 724.3868 | 732.4010 | IATPR-GAAPPGQK (SEQ ID NO: 166) |
| | | | | IATPR-GAAPPGQK-GQANA (SEQ ID NO: 167) |
| | | | | GAAPPGQK-GQANA (SEQ ID NO: 168) |
| | GQANATR (SEQ ID NO: 73) | 716.3565 | 726.3648 | PPGQK-GQANATR (SEQ ID NO: 169) |
| | | | | PPGQK-GQANATR-IPAKT (SEQ ID NO: 170) |
| | | | | GQANATR-IPAKT (SEQ ID NO: 171) |
| | HQPGGGK (SEQ ID NO: 74) | 679.3402 | 687.3544 | TENLK-HQPGGGK(SEQ ID NO: 172) |
| | | | | TENLK-HQPGGGK-VQIIN (SEQ ID NO: 173) |
| | | | | HQPGGGK-VQIIN (SEQ ID NO: 174) |
| | HVPGGGSVQIVYKPVDLSK (SEQ ID NO: 75) | 1979.0840 | 1987.0982 | KDNIK-HVPGGGSVQIVYKPVDLSK (SEQ ID NO: 175) |
| | | | | KDNIK-HVPGGGSVQIVYKPVDLSK-VTSKC (SEQ ID NO: 176) |
| | | | | HVPGGGSVQIVYKPVDLSK-VTSKC (SEQ ID NO: 177) |
| | DNITHVPGGGNK (SEQ ID NO: 76) | 1207.5945 | 1215.6087 | KIGSL-DNITHVPGGGNK (SEQ ID NO: 178) |
| | | | | KIGSL-DNITHVPGGGNK-KIETH (SEQ ID NO: 179) |
| | | | | DNITHVPGGGNK-KIETH (SEQ ID NO: 180) |
| | DHGAEIVYK (SEQ ID NO: 77) | 1030.5083 | 1038.5225 | AKAKT-DHGAEIVYK (SEQ ID NO: 181) |
| | | | | AKAKT-DHGAEIVYK-SPVVS (SEQ ID NO: 182) |
| | | | | DHGAEIVYK-SPVVS (SEQ ID NO: 183) |
| | DEVSASLAK (SEQ ID NO: 78) | 918.4658 | 926.4800 | LATLA-DEVSASLAK (SEQ ID NO: 184) |
| | | | | LATLA-DEVSASLAK-QGL (SEQ ID NO: 185) |
| | | | | DEVSASLAK-QGL (SEQ ID NO: 186) |
| | IGSLDNITHVPGGGNK (SEQ ID NO: 189) | 1577.8161 | 1585.8304 | RVQSK-IGSLDNITHVPGGGNK (SEQ ID NO: 191) |
| | | | | RVQSK-IGSLDNITHVPGGGNK-GGSVQ (SEQ ID NO: 192) |
| | | | | IGSLDNITHVPGGGNK-GGSVQ (SEQ ID NO: 193) |
| | HVPGGGSVQIVYKPV (SEQ ID NO: 190) | 1535.8460 | 1543.8602 | KDNIK-HVPGGGSVQIVYKPV(SEQ ID NO: 194) |
| | | | | KDNIK-HVPGGGSVQIVYKPV-DLSKV (SEQ ID NO: 195) |
| | | | | HVPGGGSVQIVYKPV-DLSKV (SEQ ID NO: 196) |

### Proteases:

After the isolation of a protein sample, e.g. by Immuno-Precipitation, the protein sample is digested typically with a Protease although chemical cleavage of proteins may also be a useful way to digest proteins into peptides suitable for analysis by mass spectrometry. A particularly preferred protease is Trypsin, which cleaves exclusively C-terminal to Arginine and Lysine residues. Trypsin-like proteases cleave peptide bonds following a positively charged amino acid (e.g., lysine (K) or arginine (R)). This specificity is driven by the residue, which lies at the base of the enzyme's S1 pocket (generally a negatively charged aspartic acid or glutamic acid). Trypsin is a preferred choice to generate peptides because cleavage at the C-terminal side of Arginine or Lysine means that peptides all have a charge-carrying amino acid at the C-terminus of the peptide, while also having a free alpha-amino group at the N-terminus also generated by the cleavage with Trypsin. Arginine, Lysine and the alpha amino group are all readily protonated, generating ions with charges localised at either end of the peptide, which is helpful for generating identifiable ions that determine the sequence of the peptide. Trypsin also generates peptides whose masses are compatible with the optimal mass range for detection of most mass spectrometers (up to 2000 m/z) but whose average length means that they are usually uniquely identifiable. Accordingly, Trypsin is widely used resulting in the availability of efficient algorithms for the generation of databases of theoretical trypsin-generated peptides as well as public archives of mass spectrometry data from Trypsin-derived peptides. High cleavage specificity, availability, and cost are other advantages of Trypsin. Trypsin does however sometimes generate miscleavages when two or more lysine or arginine amino acids appear consecutively in a protein sequence. Trypsin may only cleave at one of these repeating charged amino acids generating what are referred to as ragged ends. It is sometimes preferable to avoid peptides that may result in ragged ends. Accordingly, it may be preferable that the peptides do not include any peptide that is bounded by KK, KR, RK or RR, either upstream of downstream in the corresponding protein sequence.

Other suitable proteases will be known to one of ordinary skill in the art. Other suitable proteases include Endoproteinase Lys-C, which cleaves specifically at the C-terminus of Lysine residues and Arg-C, which cleaves selectively at the C-terminus of Arginine residues. Like Trypsin, both Lys-C and Arg-C generate peptides with readily protonated groups at the termini of the peptide although Lys-C and Arg-C typically generate fewer and slightly longer peptides. These enzymes may be selected if it is difficult to find a suitable peptide to identify a protein using Trypsin. Alternatively, a combination of Trypsin and Endoproteinase Lys-C is used. It is reported that this combination of enzymes has a higher overall level of completeness of cleavage than Trypsin alone (Hakobyan et al., Proteomics, 19(20):e1900136, (doi: 10.1002/pmic.201900136), “Efficient Tandem LysC/Trypsin Digestion in Detergent Conditions”, 2019).

The protease may, additionally, be selected from an Aspartic Protease (e.g. Pepsin, Cathepsin D, Cathepsin E, Napsin-A, Nepenthesin, Presenilin, Renin (Chymosin)), a Glutamic Protease (e.g. Scytalidoglutamic Peptidase (Eqolisin), Aspergilloglutamic Peptidase), a Metalloprotease (e.g. an ADAM or a Matrix Metalloproteinase), or an Asparagine Peptide Lyase. Preferably the protease is Asp-N (e.g. Asp-N, Sequencing Grade that is commercially available from Promega, catalogue number: V1621). The inventors observed that the peptides that are unique for CNS Tau generated by cleavage of CNS subtypes of the MAPT protein by trypsin give rise to large peptides that may not be detected with high sensitivity as shown in Table 1. Accordingly, the inventors made the critical observation that cleavage at aspartic acids after cleavage with Trypsin would generate smaller peptides that are still unique for CNS Tau but which will be detected with higher sensitivity as shown in Table 3.

However, miscleavage by proteases is a factor for failure of protein identification, ambiguous protein identification or inaccurate quantification although various methods can be used to either improve cleavage or compensate for incomplete cleavage. A miscleavage can be defined as partial enzymatic protein cleavages generating peptides with internal missed cleavage sites reflecting the allowed number of sites (targeted amino acids) per peptide that were not cut. The presence of post-translational modifications (PTMs) is also a potential contributor to the problem of miscleavages. As discussed in the previous section on protein standards and reference peptides, whole protein standards or reference peptides, which must also be cleaved by the selected protease, can be used to improve quantitative accuracy of LC-MS/MS SRM analysis and compensate for miscleavages. Denaturants and surfactants can also help disrupt protein structure making proteins more accessible to proteases improving overall digestion efficiency.

### Liquid Chromatography Tandem Mass Spectrometry:

In most applications of the present invention, peptides derived from the target proteins of interest are analysed by liquid chromatography mass spectrometry.

### Liquid Chromatography:

Preferably, one or more LC purification steps are performed prior to a subsequent MS/MS analysis step. Traditional LC analysis relies on the chemical interactions between sample components and column packing materials, where laminar flow of the sample through the column is the basis for separation of the analyte of interest from the test sample. One of ordinary skill in the art will understand that separation in such columns is a diffusional process. A variety of column packing materials are available for chromatographic separation of samples, and selection of an appropriate separation protocol is an empirical process that depends on the sample characteristics, the analyte of interest, the interfering substances present and their characteristics, etc. Various packing chemistries can be used depending on the needs (e.g., structure, polarity, and solubility of compounds being purified). It may be preferable for some samples that the columns are based on polar interactions, ion exchange (both cation and anion), hydrophobic interactions, e.g. phenyl, C-2, C-8, C-18 columns, polar coating on porous polymer, or mixed mode separations that combine some of the features of each of these separation modalities. During chromatography, the separation of materials is effected by variables such as choice of eluant (also known as a “mobile phase”), choice of gradient elution and the gradient conditions, temperature, etc. An analyte may be purified by applying a sample to a column under conditions where the analyte of interest is reversibly retained by the column packing material, while one or more other materials are not retained. In this scenario, a first mobile phase condition can be employed where the analyte of interest is retained by the column, and a second mobile phase condition can subsequently be employed to remove retained material from the column, once the non-retained materials are washed through. Alternatively, an analyte may be purified by applying a sample to a column under mobile phase conditions where the analyte of interest elutes at a differential rate in comparison to one or more other materials including other analytes. LC methods can ensure that multiple peptide analytes according to this invention are separated from each other to maximise specificity and sensitivity. As discussed above, such procedures may enrich the amount of one or more peptides of interest relative to one or more other components of the sample including other peptides of interest.

### Selected Reaction Monitoring:

In this invention, proteins are identified by targeted mass spectrometric analysis of proteotypic peptides typically using a technique referred to as Selected Reaction Monitoring (SRM). SRM mass spectrometry is capable of highly sensitive and accurate protein quantification based on the quantification of proteolytic peptides. In terms of clinical utility, mass spectrometry-based assays are often compared to immunoassays (e.g., Enzyme-Linked Immunosorbent Assay, or ELISA), which have the ability to quantify specific analytes in large sample sets (e.g., 96 or 384 samples in parallel microtitre plate-based format). Until recently, mass spectrometry-based protein assays were not able to match this sample throughput or the sensitivity of immunoassays. However, considerable time and expense is required to generate and characterize antibodies required for immunoassays particularly as two antibodies are typically need per protein for an ELISA due to the limitations on the specificity of many antibodies. Increasingly sensitive LC-MS/MS assays, may now surpass immunoassays such as ELISA in the rapid development of clinically useful, sensitive, multiplexed protein assays by offering higher specificity through detection of highly unique peptide mass and MS/MS transition combinations combined with high sensitive and the ability to multiplex large numbers of protein targets in a single assay.

Antibodies may also be used with Targeted LC-MS/MS assays to enrich for a protein of interest but typically only one antibody per protein is needed as any non-specific binding of other proteins to the antibody is not a problem due to the highly specific isolation and detection of peptide ions from the protein of interest during LC-MS/MS analysis. In addition, antibodies or other ligands do not need to be highly optimised for specificity towards a particular target protein as the use of highly selective LC-MS/MS SRM assays are sufficient to specifically identify the target protein even if it is pulled down with a range of other proteins.

Targeted LC-MS/MS such as SRM are highly selective methods of tandem mass spectrometry, which have the potential to effectively filter out all molecules and contaminants except the desired analyte(s), which can then be identified and quantified. This is particularly beneficial if the analysis sample is a complex mixture, which may comprise several isobaric species within a defined analytical window as many analytes can be resolved uniquely in a complex background.

LC-MS/MS mass spectrometry typically involves the isolation of gas phase ions, proteolytic peptide ions for the purposes of this invention, referred to as precursor ions. Isolated precursor ions are then subjected to fragmentation to generate fragment ions referred to as product ions and the combination of the mass-to-charge ratios (m/z) of the precursor and product ions is sufficient to identify a peptide and, if the peptide is unique to a particular protein, the identification of the peptide will also identify the protein. A particular combination of a precursor ion m/z and a product ion m/z is referred to as a transition. The transition ions observed in an LC-SRM-MS spectrum result from several different factors, which include, but are not limited to, the primary sequence, the amount of internal energy, the means of introducing the energy, and charge state. Transition ions must carry at least one charge to be detected.

Fragmentation can be induced between the different stages of mass analysis. There are many methods used to fragment the ions and these can result in different types of fragmentation and thus different information about the structure and composition of the molecule.

LC-MS/MS methods may be carried out on a triple or tandem quadrupole mass spectrometer, which, as is known in the art, typically includes three quadrupole rod sets although the second quadrupole may be replaced with other ion transmission and ion fragmentation arrangements.

A first stage of mass selection is performed in the first quadrupole rod set, where a single precursor ion may be selected (gated) and then selectively transmitted to be fragmented in the second quadrupole rod set, which could also be a travelling wave based collision cell. The resultant product ions generated by fragmentation in the second quadrupole are conveyed to the third quadrupole rod set, which performs a second stage of mass selection. A specific product ion is transmitted through the third quadrupole rod set to be measured by a detector, which generates a signal representative of the numbers of selectively transmitted product ions. Quadrupoles act as ion gates allowing passage of an ion with a pre-selected m/z that is determined by the Radio-Frequency (RF) and Direct Current (DC) potentials applied to the quadrupoles. The RF and DC potentials applied to the first and third quadrupoles are tuned to select (respectively) precursor and product ions that have m/z values lying within narrow specified ranges. The combination of precursor ion m/z and product ion m/z gating potentials are referred to as a transition as the pair of m/z settings allow the transition (and transformation) of a precursor ion to a product ion transmitting the product ion to the detector. By specifying the appropriate transitions (m/z values of precursor and product ions), a peptide corresponding to a targeted protein may be identified and quantified with high degrees of sensitivity and selectivity. This targeted LC-MS/MS method is typically referred to as Selected Reaction Monitoring (SRM) or Multiple Reaction Monitoring (MRM). These terms are interchangeable and SRM will be primarily used in this disclosure. Signal-to-noise ratio in SRM is often superior to conventional tandem mass spectrometry (MS/MS) experiments that do not selectively target (filter) particular analytes but rather aim to survey all analytes in the sample.

While triple quadrupole mass spectrometry platforms, operated in a scheduled SRM mode, are a preferred platform for the assays of this invention due to their wide availability, robustness, their high dynamic range and linearity of signal detection for the purposes of quantification, other mass spectrometry platforms also suitable for the assays of this invention. However, different mass spectrometry platforms can also be used to measure the same set of analytes such as high-resolution mass spectrometry platforms (e.g. Sciex 6600 TripleTOF, Thermo Orbitrap or Waters QTOF-type instruments) where Parallel Reaction Monitoring modes could be used for measurements instead.

However, irrespective of the physical instrument used, the underlying methods of peptide selection to allow for identification of the parent proteins are common to all the instruments that could be used. Proteotypic peptides and their identifying fragment ions must be selected from among all the possible peptides that may generated by proteolytic cleavage of a target protein. Accordingly, provided herein is a method for developing peptides and transitions for a plurality of proteins for use in SRM assays. Preferably, the assay involves the analysis of a single sample containing all analytes of interest (e.g., a proteolytic digest of plasma proteins). This disclosure provides methods for developing peptides and transitions for a single sample SRM mass spectrometry (LC-MS/MS) assay for diagnosis of a neurodegenerative disease, generally comprising the steps of selecting a set of target proteins; identifying representative proteolytic and proteotypic peptides for each protein according to the set of criteria listed below; identifying representative transitions for each peptide according to these criteria; and selecting the optimum peptides per protein and the optimum transitions per peptide.

Criteria for Selection of Peptides for Use in this Invention:

Accordingly, provided herein are criteria for selecting peptides and transitions from the set of protein subtypes of Microtubule Associated Protein Tau for use in a single sample targeted LC-MS/MS SRM assay, including the steps of providing a set of MAPT protein subtypes; selecting peptides, generating transitions for each protein; performing collision energy optimization on the transitions; selecting peptides with transitions showing the greatest peak areas of their transitions; selecting a set of transitions for each peptide, wherein the transitions for each peptide have one of the four most intense b or y transition ions; the transitions for each peptide having m/z values of at least 30 m/z above or below those of the precursor ion; the transitions for each peptide do not interfere with transitions from other peptides; and the transitions represent transitions due to breakage of peptide bond at different sites of the protein. Exemplary transitions for use with this invention are listed in Table 4.

For practical purposes, each selected peptide in the set of peptides has a monoisotopic mass of 500-5000 Da; and preferably does not contain a cysteine or a methionine. It should be noted that the absence of cysteine or methionine is not a strict requirement but generally preferred as cysteine needs to be blocked and it can be challenging to drive to completion the blocking of cysteine while methionine is prone to oxidation which may alter the associated peptide mass and oxidised methionine may split the signal from a peptide between oxidised and non-oxidised forms. However, it may be necessary if no other peptides are available, that each selected peptide may contain cysteine or methionine. Where a methionine-containing peptide is used, one or more standards for the oxidized form(s) of the peptide may be included in addition to any standards for the unoxidized form of the peptide.

Preferably, selection of peptides avoids any peptide that is bounded by KK, KR, RK or RR (either at the N-terminus of the peptide or at the C-terminus of the peptide) in the corresponding protein sequence although again, one of ordinary skill in the art will be aware that this is not a strict requirement as it may be that for some proteins no suitable peptides can be found that are not bounded by KK, KR, RK or RR or that peptides bound by KK, KR, RK or RR are particularly sensitive overcoming any loss of signal due to incomplete cleavage.

Preferably, each peptide of said set of peptides is unique to the corresponding protein.

Typically, the two best peptides per protein and the two or three best transitions per peptide are selected based on experimental data resulting from LC-MS/MS analysis of one or more of the following experimental samples: a biological disease sample, a biological control sample, or a mixture of synthetic peptides of interest.

Typically, the top two transitions per peptide are selected according to one or more of the following criteria the transitions exhibit the largest peak areas measured in either of the two biological experimental samples; the transitions are not interfered with by other ions; the transitions do not exhibit an elution profile that visually differs from those of other transitions of the same peptide; or the transitions are not beyond the detection limit of both of the two biological experimental samples.

Alternatively, the top two peptides per protein are selected according to one or more of the following criteria: one or more peptides exhibit two transitions and represent the largest combined peak areas of the two transitions based on experimental results, e.g. from analysis of a mixture of synthetic peptides; or one or more peptides exhibit one transition and represent the largest combined peak areas of the two transitions. Alternatively, three, four, five or six peptides may be selected per protein and the three, four, five or six best transitions per peptide are selected and optimized. It is not a requirement of the invention that the same number of peptides must be selected per protein or that the same number of transitions must be selected per peptide.

MS/MS spectra are preferably acquired for synthetic versions of peptides of interest, using a suitable instrument to empirically determine the best peptides (typically peptides with the highest intensity relative to other peptides from the same protein where all the peptides are present in the same molar quantity). One or more spectra for the doubly and the triply charged precursor ions are collected for each of the identified peptides, retention time is recorded for the most intense b- or y-transition ions. The selected transition ions possessed m/z values are preferably 30 Thomsons above or below those of the precursor ions; they did not interfere with other synthetic peptides; and they were transition ions due to breakage of peptide bonds at different sites in the peptide.

In general, peptides are selected that are unique to the protein of interest, referred to as proteotypic peptides. Proteotypic peptides are selected that match only one protein or protein family including analogues of the one protein, when searched in protein databases such as UniProt. Further, when peptides are intended to quantify the total amount of a given protein, then peptides that are observed in post-translational modified forms are typically not selected. Validity of peptides may be tested ‘in silico’ before being assessed by physical experiments. Databases of plasma proteomes may be searched to determine whether a particular protein of interest has been detected in blood previously and if detected by mass spectrometry, which peptides from the protein of interest have been detected in blood. This gives a good indication of the suitability of a peptide for analysis by targeted proteomics. Also databases of good quality peptide mass spectra may be searched such as PeptideAtlas (Deutsch, Methods Mol Biol, 604:285-96, “The PeptideAtlas Project”, 2010). Peptides that have been detected in human blood in previous studies and gave good quality mass spectra are favoured. If these selection criteria do not result in a sufficient number of peptides, rules may be relaxed in a step-wise manner to allow a greater number of peptides until a sufficient number of proteotypic peptides are selected. When making synthetic peptides for use with this invention, the purity of the synthesized peptides is preferably >75% and for Stable Isotope Internal Standards (SIIS) the purity is preferably >98%.

Alternatively, each set of peptides is prioritized according to one or more of the following ordered set of criteria: (a) unique peptides first, then non-unique; (b) peptides with no observed post-translational modifications first, then those observed with post-translational modifications; (c) peptides within the mass range 800-3500 Da first, then those outside of 800-3500 Da; and (d) sorted by decreasing number of variant residues. Alternatively, each set of peptides is prioritized according to all of the ordered set of criteria.

If an insufficient percentage of the synthetic peptides that are analysed for a given protein are suitable for use in an assay according to the invention, the selection of peptides may be repeated with other peptides derived from the protein of interest if further peptides are available.

LC-MS/MS mass spectrometry involves the fragmentation of gas phase ions and occurs between the different stages of mass analysis. There are many methods used to fragment the ions and these can result in different types of fragmentation and thus different information about the structure and composition of the molecule. The ion transitions observed in an LC-SRM-MS spectrum result from several different factors, which include, but are not limited to, the primary sequence, the amount of internal energy, the means of applying the energy, and charge state. Product ions must carry at least one charge to be detected. An ion is categorized as either a, b or c if the charge is on a fragment comprising the original N terminus of the peptide, whereas the ion is categorized as either x, y or z if the charge is on a fragment comprising the original C terminus of the peptide. A subscript indicates the number of residues in the fragment ion (e.g., one peptide residue in x₁, two peptide residues in y₂, and three peptide residues in z₃, etc.).

In a generic peptide repeat unit represented —N—C(O)—C—, an x ion and an a ion resulting from cleavage of the carbonyl-carbon bond (i.e., C(O)—C). The ‘x-ion’ is an acylium ion, and the ‘a-ion’ is an iminium ion. A ‘y-ion’ and a ‘b-ion’ result from cleavage of the carbonyl-nitrogen bond (i.e., C(O)—N, also known as the amide bond). In this case, the y ion is an ammonium ion and the b ion is typically an oxazolone ion. Finally, a ‘z-ion’ and a ‘c-ion’ result from cleavage of the nitrogen-carbon (i.e., C—N) bond. The ‘z-ion’ is a carbocation and the ‘c-ion’ is an ammonium ion.

Superscripts are sometimes used to indicate neutral losses in addition to the backbone fragmentation, for example, * for loss of ammonia and ^{∘} for loss of water. In addition to protons, c-ions and y-ions may abstract an additional proton from the precursor peptide. In electrospray ionization, tryptic peptides may carry more than one charge.

Internal fragment ions arise from double backbone cleavage—these are typically difficult to predict in silico and are usually identified empirically. These may be formed by a combination of b-type and y-type cleavage (i.e., cleavage producing b and y ions). Internal cleavage ions may also be formed by a combination of a-type and y-type cleavage. An internal product ion with a single side chain formed by a combination of a-type and y-type cleavage is called an iminium ion (sometimes also referred to as an imonium or immonium ion). These ions are labeled with the one letter code for the corresponding amino acid.

Low energy CID (i.e., collision induced dissociation in a triple quadrupole or an ion trap) involves the fragmentation of a peptide carrying a positive charge, primarily along its backbone, to generate primarily a-, b- and y-ions.

Post-translational modification (PTM) is the chemical modification of a protein after its translation. It can include any modification following translation, including cleavage. It is one of the later steps in protein biosynthesis, and thus gene expression, for many proteins. It is usually desirable to avoid such peptides for the purpose of protein identification for a number of reasons, 1) since SRM methods typically rely on synthetic heavy isotope peptide standards, and since chemical synthesis of some PTMs such as some sugar modifications are either difficult or impossible currently, these are best avoided, unless a suitable internal standard can be generated 2) PTMs are typically sub-stoicheometric, i.e. only a small percentage of a specific modifiable site in a protein is actually modified and detection of the PTM can be challenging.

However, some PTMs such as phosphorylated amino acids are accessible by chemical synthesis and can be detected with enrichment of the peptides. Thus, when the PTM itself is important to the assay, PTM modified peptides can be selected. In the context of the present disclosure, phosphorylated peptides from Tau have been selected as key targets in the assay.

Typically, the top two or more transitions per peptide are selected according to one or more of the following criteria: (1) the transitions exhibit the largest peak areas measured in either of the two biological experimental samples; (2) the transitions are not interfered with by other ions; (3) the transitions do not exhibit an elution profile that visually differs from those of other transitions of the same peptide; (4) the transitions are not beyond the detection limit of the chosen instrument; (5) the transitions do not exhibit interferences.

### Assay Parameters:

The following parameters are used to specify an LC-MS/MS assay of a protein under a particular LC-MS/MS system: (1) a tryptic peptide of the protein; (2) the retention time (RT) of the peptide on the LC column for a given chromatographic gradient; (3) the declustering potential used to ionize the precursor ion; (4) the m/z value of the peptide precursor ion; (5) m/z value of a fragment ion generated from the peptide precursor ion; and (6) the collision energy (CE) used to fragment the peptide precursor ion that is optimized for the particular peptide.

It may be preferred that the two best peptides per protein and the two best transitions per peptide are selected based on empirical data resulting from LC-MS/MS analysis of a synthetic version of the peptide.

### Heavy Isotope Internal Standards:

To facilitate accurate quantification of the peptide transitions by the methods disclosed herein, a set of isotopically-labeled synthetic versions of the peptides of interest may be added in known amounts to the sample for use as internal standards. Since the isotopically-labeled peptides have physical and chemical properties identical to the corresponding surrogate peptide, they co-elute from the chromatographic column and are easily identifiable on the resultant mass spectrum. The addition of the labeled reference peptide standards may occur before or after proteolytic digestion although digestion standards that are added before proteolytic digestion may off better quantitative accuracy. Methods of synthesizing isotopically-labeled reference peptides will be known to those of skill in the art. In general it is preferred that the experimental samples contain internal standard reference peptides. However, one of ordinary skill in the art will be aware that it is possible to utilize external standards or other expedients for peptide quantification.

For the mass spectrometric analysis of a particular peptide, the quantities of the peptide transitions in the sample may be determined by integration of the relevant mass spectral peak areas, as known in the prior art. When isotopically-labeled internal standard reference peptides are used, as described above, the quantities of the peptide transitions of interest are established via an empirically-derived or predicted relationship between peptide transition quantity (which may be expressed as concentration) and the area ratio of the peptide transition and internal standard peaks at specified transitions.

### Example 1—LC-MS/MS Analysis of Tryptic Digests of Tau Isoforms

A number of different LC-MS/MS methods were developed for the detection of Tau (MAPT; Uniprot ID P10636) isoforms in body fluids such as Blood Plasma and CSF.

### Preparation of Recombinant Protein Samples:

The proteins listed in Table 9 were sourced from the suppliers shown and were delivered in 100 μg aliquots. Sources for enzymes for digestion (Trypsin and AspN) and digestion enhancer (Rapigest) are also listed in Table 9.

TABLE 9
List of reagents and consumables for the assay:

| Consumable | Supplier | Catalogue Number |
|---|---|---|
| Tau-441 Recombinant | rPeptide | T-1001-2 |
| Protein (CNS-Tau) | | |
| Tau-410 Recombinant | rPeptide | T-1002-2 |
| Protein (CNS-Tau) | | |
| Recombinant Big Tau (PNS-Tau) | Genscript | Custom synthesis |
| Rapigest | Waters | Ref#186001861 |
| Asp-N | Promega | Ref# T1621-2 ug |
| Trypsin gold | Promega | Ref# V5280 |

Stock solutions of each recombinant protein were prepared at 0.1 mg/mL in 0.1% (w:w) Bovine Serum Albumin (BSA) in Phosphate Buffer Saline (PBS). Rapigest Buffer was made up according to manufacturer's instructions: 1 mL of 50 mM Ammonium Bicarbonate (AmBic) buffer was added to a 1 mg vial of Rapigest; Final Conc: 0.1% (mg/L) of Rapigest. AspN solution: 2 μg vial of AspN (Promega) were dissolved with 50 μl of double-distilled water. Trypsin Gold solution: the 100 μg vial of Trypsin gold (Promega) from the manufacturer was made up with 1 mL of 50 mM acetic acid (2.87 uL of glacial acetic acid to 1 mL of double-distilled water). Tubes for digestion were made up according to Table 10.

TABLE 10
Digestion conditions:

| | Tau 410 | Tau 441 | Big Tau | | |
|---|---|---|---|---|---|
| | Stock | Stock | Stock | PBS | Rapigest |
| Tube Label | solution | solution | solution | buffer | Buffer |
| Tau 410 DD-C | 10 μl | — | — | 30 ul | 40 ul |
| Tau 410 DD-CN | 10 ul | — | — | 30 ul | 40 ul |
| Tau 441 DD-C | — | 10 ul | — | 30 ul | 40 ul |
| Tau 441 DD-CN | — | 10 ul | — | 30 ul | 40 ul |
| Big Tau DD-C | — | — | 10 ul | 30 ul | 40 ul |
| Big Tau DD-CN | — | — | 10 ul | 30 ul | 40 ul |

### Reduction and Alkylation of Recombinant Proteins:

Dithiothreitol (DTT) solution: 400 mM DTT was prepared by dissolving 61.6 mg of DTT in 1 mL of ddH2O. Iodoacetamide (IAA) solution: 150 mm IAA was prepared by dissolving 27.8 mg of iodoacetamide in 1 mL of 50 mM AmBic.

1 μl of dithiothreitol (DTT) solution was added to all 6 samples from Table 1 and vortexed well (final concentration of DTT should be 5 mM). Samples were incubated at 60° C. for 30 minutes with mixing. The samples were allowed to cool for 5 minutes and then 8 μl of iodoacetamide solution (final concentration of Iodoacetamide should be ~14 mM) was added. The samples were incubated with mixing for 30 minutes in the dark. Unreacted iodoacetamide was quenched by adding a further 1 μl of dithiothreitol (DTT) solution and incubate for a further 15 min.

TABLE 11
Protein Digestion Conditions:

| Tube Label | Trypsin Gold | AspN |
|---|---|---|
| Tau 410 DD-C | 10 μl | — |
| Tau 410 DD-CN | 10 μl | 10 μl |
| Tau 441 DD-C | 10 μl | — |
| Tau 441 DD-CN | 10 μl | 10 μl |
| Big Tau DD-C | 10 μl | |
| Big Tau DD-CN | 10 μl | 10 μl |

Protease solutions were then added to the 6 recombinant protein samples according to Table 11 The samples were mixed gently and then centrifuged briefly to collect all liquid to bottom of the tubes. The 6 samples were then incubated at 37° C. with shaking for 4 hours. Following incubation, 5 μl of 10% aqueous TFA solution was added to all samples to give final [TFA] of 0.5%, which degraded the Rapigest Surfactant. Samples were incubated at 37° C. for 30 minutes to precipitate Rapigest and then spin at 13,000 rpm (or high speed) for 10 minutes to pellet the Rapigest precipitate. Samples were transferred to clean appropriately labelled Lo Bind Eppendorfs and dried down. 25 μL 0.1% aqueous formic acid was added to each of the dried samples, and then these were vortexed well to redissolve the digests. The samples were then centrifuged for 10 minutes at 13000 rpm. The supernatant was split into 2 separate sets of LCMS vials (10 ul for each vial) and the samples were cleaned up with ZipTips® (EMDMillipore) following the manufacturer's instructions.

### Data Dependent Analysis (DDA) of Recombinant Protein Digests:

In the first instance the digested protein samples were analysed by DDA using high-sensitivity nanoflow chromatography to check the digests and identify as many peptides as possible. The desalted peptide digests were analysed by low pH reverse phase liquid chromatography (RP-LC) on a Thermo Ultimate 3000 Rapid Separation Liquid Chromatography (RSLC) nanoflow LC system coupled to a Thermo Exploris 480 Orbitrap. Samples were first loaded onto a trapping column: Acclaim PepMap100 C18 LC Column (5 mm A~0.3 mm i.d., 5 μm, 100 Å, Thermo Fisher Scientific) at a flow rate of 10 μL/min maintained at 50° C. in a column oven. This was followed by analytical separation on a 200 cm pPAC Generation 1 C18 nanoflow column with a bed width of 318 μm and a pillar height of 18 μm using the gradient shown in Table 12 (Solvent B is 80% Acetonitrile, 0.05% Formic acid). 5 μl of protein digest from each sample was loaded on column for each LC-MS/MS run. The Thermo Exploris 480 Orbitrap was operated in Data Dependent Acquisition mode.

TABLE 12
LC Gradient for DDA analysis:
200 cm uPAC Neo column

| Time (min) | Flow rate (μL/min) | % B |
|---|---|---|
| 0 | 0.25 | 1 |
| 0.1 | 0.25 | 2 |
| 44.1 | 0.25 | 22.5 |
| 60.1 | 0.25 | 45 |
| 60.6 | 0.25 | 99 |
| 68 | 0.25 | 99 |

Table 13 shows the intensity and retention times of the peptides from Tables 1 and 2 that were identified in the Trypsin-only digests of the recombinant proteins. Similarly, Table 14 shows the intensity and retention times of the peptides from Tables 2 and 3 that were identified in the Trypsin/AspN double digests of the recombinant proteins. Note that the highest intensity measurement for each peptide from the different digests is shown as some peptides would not be expected to be in all samples. Tables 13 and 14 provide the necessary data to select peptides for targeted assays and provides their retention times. These retention times could be used with the longer analytical LC column and a longer gradient although to maximize throughput data from a shorter column with shorter gradients is shown in the next section (Tables 16 and 17).

TABLE 13
Peptide Intensities and Retention Times from DDA analysis of Tryptic Digests:

| Tau | | | Retention | |
|---|---|---|---|---|
| Isoform | Sequence | Comments | time (Min) | Intensity |
| CNS-Tau | AEEAGIGDTPSLEDEAAGHVTQAR (SEQ ID NO: 1) | | 87.409 | 2.82E+05 |
| CNS-Tau | QAAAQPHTEIPEGTTAEEAGIGDTPSLE | | 93.341 | 3.07E+10 |
| | DEAAGHVTQAR (SEQ ID NO: 2) | | | |
| CNS-Tau | STPTAEAEEAGIGDTPSLEDEAAGHVTQAR | Not present in the | — | — |
| | (SEQ ID NO: 3) | available recombinant | | |
| | | proteins | | |
| CNS-Tau | VQIVYKPVDLSK (SEQ ID NO: 4) | | 90.675 | 2.91E+10 |
| CNS-Tau | EAGIGDTPSLEDEAAGHVTQAR (SEQ ID NO: 5) | Not present in the | | |
| | | available recombinant | | |
| | | proteins | | |
| PNS-Tau | QPSGTGPEDTEGGR (SEQ ID NO: 6) | | 50.492 | 1.37E+07 |
| PNS-Tau | DVDESSPQDSPPSK (SEQ ID NO: 7) | | 50.427 | 5.32E+07 |
| PNS-Tau | ASPAQDGRPPQTAAR (SEQ ID NO: 8) | | 50.778 | 1.20E+10 |
| PNS-Tau | EATSIPGFPAEGAIPLPVDFLSK (SEQ ID NO: 9) | | 99.046 | 5.36E+09 |
| PNS-Tau | VSTEIPASEPDGPSVGR (SEQ ID NO: 10) | | 79.158 | 5.49E+09 |
| PNS-Tau | GQDAPLEFTFHVEITPNVQK (SEQ ID NO: 11) | | 97.313 | 2.27E+09 |
| PNS-Tau | GPSLGEDTK (SEQ ID NO: 12) | | 54.167 | 9.64E+06 |
| PNS-Tau | EADLPEPSEK (SEQ ID NO: 13) | | 66.343 | 4.61E+09 |
| Total-Tau | LQTAPVPMPDLK (SEQ ID NO: 14) | | 97.588 | 4.78E+10 |
| Total-Tau | TDHGAEIVYK (SEQ ID NO: 15) | | 61.321 | 1.59E+10 |
| PNS-Tau | VVQEGFLR (SEQ ID NO: 16) | | 84.946 | 6.19E+09 |
| PNS-Tau | HAPELLK (SEQ ID NO: 17) | | 66.319 | 1.94E+09 |
| PNS-Tau | HQLLGDLHQEGPPLK (SEQ ID NO: 18) | | 91.273 | 1.53E+10 |
| PNS-Tau | EQAHSEEHLGR (SEQ ID NO: 19) | | 51.139 | 1.50E+07 |
| PNS-Tau | AAFPGAPGEGPEAR (SEQ ID NO: 20) | | 78.424 | 7.89E+09 |
| Total-Tau | GAAPPGQK (SEQ ID NO: 21) | Present only in | — | — |
| | | miscleaved larger | | |
| | | peptides | | |
| Total-Tau | GQANATR (SEQ ID NO: 22) | Present only in | — | — |
| | | miscleaved larger | | |
| | | peptides | | |
| Total-Tau | HQPGGGK (SEQ ID NO: 23) | Present only in | — | — |
| | | miscleaved larger | | |
| | | peptides | | |
| Total-Tau | HVPGGGSVQIVYKPVDLSK (SEQ ID NO: 24) | | 90.34 | 4.13E+10 |
| Total-Tau | IGSLDNITHVPGGGNK (SEQ ID NO: 187) | | 82.798 | 6.93E+10 |

TABLE 14
Peptide Intensities and Retention Times from DDA analysis of Trypsin/AspN Digests:

| Tau | | | Retention | | |
|---|---|---|---|---|---|
| Isoform | Sequence | Comment | (Min) | Time | Intensity |
| PNS-Tau | VVQEGFLR (SEQ ID NO: 16) | | 86.992 | | 3.66E+09 |
| PNS-Tau | HAPELLK (SEQ ID NO: 17) | | 67.55 | | 1.52E+09 |
| PNS-Tau | HQLLGDLHQEGPPLK (SEQ ID NO: 18) | | 83.117 | | 2.30E+08 |
| PNS-Tau | EQAHSEEHLGR (SEQ ID NO: 19) | | 52.32 | | 6.60E+06 |
| PNS-Tau | AAFPGAPGEGPEAR (SEQ ID NO: 20) | | 80.314 | | 3.80E+09 |
| Total-Tau | GAAPPGQK (SEQ ID NO: 21) | Present as part of larger | — | | — |
| | | miscleaved peptides | | | |
| Total-Tau | GQANATR (SEQ ID NO: 22) | Present as part of larger | — | | — |
| | | miscleaved peptides | | | |
| Total-Tau | HQPGGGK (SEQ ID NO: 23) | Present as part of larger | — | | — |
| | | miscleaved peptides | | | |
| Total-Tau | HVPGGGSVQIVYKPV | | 84.876 | | 8.81E+07 |
| | (SEQ ID NO: 188) | | | | |
| CNS-Tau | DEAAGHVTQAR (SEQ ID NO: 25) | | 45.063 | | 2.46E+06 |
| CNS-Tau | VQIVYKPV (SEQ ID NO: 26) | | 85.749 | | 4.03E+08 |
| PNS-Tau | DEAAGHVTQEPESGK (SEQ ID NO: 27) | | 50.591 | | 6.06E+09 |
| PNS-Tau | DTEGGR (SEQ ID NO: 28) | Not detected (too small) | — | | — |
| PNS-Tau | DLHQEGPPLK (SEQ ID NO: 29) | | 67.35 | | 4.53E+09 |
| PNS-Tau | DGRPPQTAAR (SEQ ID NO: 30) | | 47.355 | | 0.00E+00 |
| PNS-Tau | DGPSVGR (SEQ ID NO: 31) | Present as part of larger | — | | — |
| | | miscleaved peptides | | | |
| PNS-Tau | DAPLEFTFHVEITPNVQK | | 98.316 | | 6.07E+08 |
| | (SEQ ID NO: 32) | | | | |
| PNS-Tau | DLPEPSEK (SEQ ID NO: 33) | | 62.949 | | 3.60E+09 |
| Total-Tau | DNITHVPGGGNK (SEQ ID NO: 34) | | 55.292 | | 7.91E+08 |
| Total-Tau | DHGAEIVYK (SEQ ID NO: 35) | | 59.873 | | 2.97E+08 |
| Total-Tau | DEVSASLAK (SEQ ID NO: 36) | | 63.622 | | 1.08E+10 |

Using the data shown, one of ordinary skill in the art would be able operate the same mass spectrometer and RP-LC setup in a targeted mode, e.g. Parallel Reaction Monitoring (PRM), to routinely detect the proteins in Tables 13 and 14.

### Data Independent Analysis (DIA) of Recombinant Protein Digests:

In a second assay, the recombinant protein digests were analysed by low pH RP-LC on the same Thermo Ultimate 3000 Rapid Separation Liquid Chromatography (RSLC) nanoflow LC system coupled to a Thermo Exploris 480 Orbitrap. Samples were first loaded onto a trapping column: Acclaim PepMap100 C18 LC Column (5 mm A~0.3 mm i.d., 5 μm, 100 Å, Thermo Fisher Scientific) at a flow rate of 10 μL/min maintained at 50° C. in a column oven. This was followed by analytical separation on a 5.5 cm pPAC Neo C18 nanoflow column with a bed width of 1850 μm and a pillar height of 25 μm using the gradient shown in Table 15. 5 μl of protein digest from each sample was loaded on column for each LC-MS/MS run. The Thermo Exploris 480 Orbitrap was operated in Data Independent Acquisition mode. This column enables shorter LC gradients and faster run times so greater throughput overall.

TABLE 15
LC Gradient for DDA analysis:
5.5 cm μPAC Neo Column

| Time (Min) | Flow rate (μL/min) | % B |
|---|---|---|
| 0 | 1 | 3 |
| 3 | 1 | 15 |
| 10 | 1 | 35 |
| 10.5 | 1 | 65 |
| 11 | 1 | 99 |
| 13 | 1 | 99 |
| 13.1 | 1 | 3 |
| 28 | 1 | 3 |

Table 16 shows the intensity and retention times of the peptides from Tables 1 and 2 that were identified in the Trypsin-only digests of the recombinant proteins. Table 17 shows the intensity and retention times of the peptides from Tables 2 and 3 that were identified in the Trypsin/AspN double digests of the recombinant proteins. Again, note that the highest intensity measurement for each peptide from the different digests is shown as some peptides would not be expected to be in all samples. Using the data shown, one of ordinary skill in the art would be able operate the same mass spectrometer and RP-LC setup in a targeted mode, e.g. Parallel Reaction Monitoring (PRM), to routinely detect the proteins in Tables 16 and 17.

TABLE 16
Peptide Intensities and Retention Times from DIA analysis of Trypsin-only Digests:

| Tau | | | Retention | |
|---|---|---|---|---|
| Isoform | Sequence | Comments | Times | Intensity |
| CNS-Tau | AEEAGIGDTPSLEDEAAGHVTQAR | Not detected | — | — |
| | (SEQ ID NO: 1) | | | |
| CNS-Tau | QAAAQPHTEIPEGTTAEEAGIGDTPSLED | | 13.392 | 4.57E+09 |
| | EAAGHVTQAR (SEQ ID NO: 2) | | | |
| CNS-Tau | STPTAEAEEAGIGDTPSLEDEAAGHVT | Not present in | — | — |
| | QAR (SEQ ID NO: 3) | available | | |
| | | recombinants | | |
| CNS-Tau | VQIVYKPVDLSK (SEQ ID NO: 4) | | 14.5 | 1.05E+10 |
| CNS-Tau | EAGIGDTPSLEDEAAGHVTQAR (SEQ ID NO: 5) | Not present in | — | — |
| | | available | | |
| | | recombinants | | |
| PNS-Tau | QPSGTGPEDTEGGR (SEQ ID NO: 6) | | — | — |
| PNS-Tau | DVDESSPQDSPPSK (SEQ ID NO: 7) | | 8.5693 | 1.36E+07 |
| PNS-Tau | ASPAQDGRPPQTAAR (SEQ ID NO: 8) | | 8.8563 | 6.09E+09 |
| PNS-Tau | EATSIPGFPAEGAIPLPVDFLSK (SEQ ID NO: 9) | | 14.398 | 1.31E+09 |
| PNS-Tau | VSTEIPASEPDGPSVGR (SEQ ID NO: 10) | | 11.619 | 2.85E+07 |
| PNS-Tau | GQDAPLEFTFHVEITPNVQK (SEQ ID NO: 11) | | 14.202 | 2.81E+08 |
| PNS-Tau | EADLPEPSEK (SEQ ID NO: 13) | | 9.6372 | 3.56E+08 |
| Total-Tau | LQTAPVPMPDLK (SEQ ID NO: 14) | | 12.538 | 1.39E+10 |
| Total-Tau | TDHGAEIVYK (SEQ ID NO: 15) | | 8.883 | 4.55E+09 |
| PNS-Tau | VVQEGFLR (SEQ ID NO: 16) | | 12.298 | 1.82E+09 |
| PNS-Tau | HAPELLK (SEQ ID NO: 17) | | 9.6348 | 5.78E+07 |
| PNS-Tau | HQLLGDLHQEGPPLK (SEQ ID NO: 18) | | 12.598 | 4.90E+09 |
| PNS-Tau | EQAHSEEHLGR (SEQ ID NO: 19) | | — | — |
| PNS-Tau | AAFPGAPGEGPEAR (SEQ ID NO: 20) | | 11.885 | 2.29E+09 |
| Total-Tau | GAAPPGQK (SEQ ID NO: 21) | Present only in | — | — |
| | | mis-cleaved | | |
| | | larger peptides | | |
| Total-Tau | GQANATR (SEQ ID NO: 22) | Present only in | — | — |
| | | mis-cleaved | | |
| | | larger peptides | | |
| Total-Tau | HQPGGGK (SEQ ID NO: 23) | Present only in | — | — |
| | | mis-cleaved | | |
| | | larger peptides | | |
| Total-Tau | HVPGGGSVQIVYKPVDLSK (SEQ ID NO: 24) | | 13.047 | 8.55E+09 |
| Total-Tau | IGSLDNITHVPGGGNK (SEQ ID NO: 187) | | 11.707 | 6.57E+09 |

TABLE 17
Peptide Intensities and Retention Times from DIA analysis of Trypsin/AspN Digests:

| Tau | | | Retention | |
|---|---|---|---|---|
| Isoform | Sequence | Comment | Time (Min) | Intensity |
| PNS-Tau | WVQEGFLR (SEQ ID NO: 16) | | 16.282 | 3.08E+09 |
| PNS-Tau | HAPELLK (SEQ ID NO: 17) | | 9.421 | 5.20E+07 |
| PNS-Tau | HQLLGDLHQEGPPLK (SEQ ID NO: 18) | | 12.703 | 1.53E+08 |
| PNS-Tau | EQAHSEEHLGR (SEQ ID NO: 19) | | 11.83 | 3.70E+06 |
| PNS-Tau | AAFPGAPGEGPEAR (SEQ ID NO: 20) | | 14.343 | 3.20E+05 |
| Total-Tau | GAAPPGQK (SEQ ID NO: 21) | Present as part of | — | — |
| | | larger mis- | | |
| | | cleaved peptides | | |
| Total-Tau | GQANATR (SEQ ID NO: 22) | Present as part of | — | — |
| | | larger mis- | | |
| | | cleaved peptides | | |
| Total-Tau | HQPGGGK (SEQ ID NO: 23) | Present as part of | — | — |
| | | larger mis- | | |
| | | cleaved peptides | | |
| Total-Tau | HVPGGGSVQIVYKPV (SEQ ID NO: 188) | | 10.916 | 3.04E+09 |
| CNS-Tau | DEAAGHVTQAR (SEQ ID NO: 25) | | 8.411 | 1.42E+06 |
| CNS-Tau | VQIVYKPV (SEQ ID NO: 26) | | 12.185 | 1.76E+08 |
| PNS-Tau | DEAAGHVTQEPESGK (SEQ ID NO: 27) | | 8.781 | 6.77E+09 |
| PNS-Tau | DTEGGR (SEQ ID NO: 28) | Not detected | — | — |
| | | (too small) | | |
| PNS-Tau | DLHQEGPPLK (SEQ ID NO: 29) | | 10.241 | 4.22E+09 |
| PNS-Tau | DGRPPQTAAR (SEQ ID NO: 30) | | 17.524 | 9.54E+04 |
| PNS-Tau | DGPSVGR (SEQ ID NO: 31) | Present as part of | — | — |
| | | larger mis- | | |
| | | cleaved peptides | | |
| PNS-Tau | DAPLEFTFHVEITPNVQK (SEQ ID NO: 32) | | 15.263 | 5.19E+08 |
| PNS-Tau | DLPEPSEK (SEQ ID NO: 33) | | 9.258 | 1.34E+09 |
| Total-Tau | DNITHVPGGGNK (SEQ ID NO: 34) | | 10.202 | 5.99E+08 |
| Total-Tau | DHGAEIVYK (SEQ ID NO: 35) | | 9.0067 | 1.14E+08 |
| Total-Tau | DEVSASLAK (SEQ ID NO: 36) | | 9.539 | 7.19E+09 |

### PRM Assay:

In a third assay, the recombinant protein digests were analysed by targeted pH RP-LC on the same instrument using the same 5.5 cm pPAC Neo column as in the previous DIA example using the same LC gradient as Table 15. In this case the Thermo Exploris 480 Orbitrap was operated in Parallel Reaction Monitoring (PRM) mode using the settings shown in Tables 18 and 19.

TABLE 18
PRM Mode scheduled peptide acquisition settings:

| | | | RT | | |
|---|---|---|---|---|---|
| | Precursor | z | Time | Window | |
| Peptide | mass | range | (min) | (min) | CE |
| IGSLDNITHVPGGGNK (SEQ ID NO: 187) | 1577.8162 | 2-3 | 13 | 2 | 30 |
| LQTAPVPMPDLK (SEQ ID NO: 14) | 1308.7112 | 2-3 | 14.2 | 2.5 | 30 |
| VQIVYKPVDLSK (SEQ ID NO: 4) | 1387.8075 | 2-3 | 13.8 | 1.5 | 30 |
| HVPGGGSVQIVYKPVDLSK | 1979.084 | 2-4 | 14 | 2 | 30 |
| (SEQ ID NO :24) | | | | | |
| ASPAQDGRPPQTAAR (SEQ ID NO: 8) | 1521.7648 | 2-3 | 7 | 2 | 30 |
| QAAAQPHTEIPEGTTAEEAGIGDTPSLE | 3954.8468 | 3-5 | 14 | 2 | 30 |
| DEAAGHVTQAR (SEQ ID NO: 2) | | | | | |
| AAFPGAPGEGPEAR (SEQ ID NO: 20) | 1325.6364 | 2-3 | 11.5 | 1.5 | 30 |
| WQEGFLR (SEQ ID NO: 16) | 946.5236 | 2-3 | 12.8 | 1.5 | 30 |

TABLE 19
PRM Mode Instrument Parameters:

| | |
|---|---|
| Parameter: | Setting |

| Expected peak width | | 12 s |
|---|---|---|
| Mass tolerance | | 15 ppm |
| Orbitrap resolution | MS1 | 60K |
| | MS2 | 15K |

| | |
|---|---|
| MS 1 range | 300-1600 |
| RF lense | 50% |
| AGC target | standard |
| Maximum injection time | auto |
| HCD collision energy | 30% |

Table 20 shows the intensity and retention times of the peptides from Table 18 that were identified in the Trypsin digests of the recombinant proteins. It can be seen that Total-Tau peptides are present in all samples. The CNS-Tau peptides are not present in the Big-Tau (Isoform-1) digest. The PNS-Tau Peptide VVQEGFLR (SEQ ID NO: 16) is present only in the Big-Tau digest as expected. The PNS-Tau peptides ASPAQDGRPPQTAAR (SEQ ID NO: 8) and AAFPGAPGEGPEAR (SEQ ID NO: 20) are both present at much higher intensities in the Big-Tau digest than in the CNS-Tau isoforms. This may indicate carry-over of these peptides between injections (requiring more stringent washing of the LC-columns and dispensing needles between injections).

TABLE 20
PRM results from measurements of Trypsin-only digests:

| | | | | | Intensity | | |
|---|---|---|---|---|---|---|---|
| | | Retention | | Pre- | Big-TAU | TAU410 | TAU-441 |
| Tau | | Time | Precursor | cursor | (Isoform- | (Isoform- | (isoform- |
| Isoform | Peptides | (mins) | M/Z | charge | 1) | 5) | 8) |
| Total-Tau | LQTAPVPMPDLK | 14.3 | 655.3629 | 2 | 1.63E+08 | 1.48E+08 | 7.22E+07 |
| | (SEQ ID NO: 14) | | | | | | |
| CNS-Tau | VQIVYKPVDLSK | 14.2 | 694.911 | 2 | 0.00E+00 | 6.45E+06 | 1.33E+05 |
| | (SEQ ID NO: 4) | | | | | | |
| Total-Tau | HVPGGGSVQIVYKPVDLSK | 14.2 | 660.7019 | 3 | 2.87E+07 | 3.74E+05 | 1.55E+08 |
| | (SEQ ID NO: 24) | | | | | | |
| PNS-Tau | ASPAQDGRPPQTAAR | 6.9 | 761.8897 | 2 | 2.17E+07 | 2.55E+04 | 1.16E+04 |
| | (SEQ ID NO: 8) | | | | | | |
| CNS-Tau | QAAAQPHTEIPEGTTA | 14.2 | 989.719 | 4 | 0.00E+00 | 1.15E+07 | 5.48E+06 |
| | EEAGIGDTPSLE | | | | | | |
| | DEAAGHVTQAR | | | | | | |
| | (SEQ ID NO: 2) | | | | | | |
| PNS-Tau | AAFPGAPGEGPEAR | 12 | 663.8255 | 2 | 2.72E+08 | 8.00E+03 | 0.00E+00 |
| | (SEQ ID NO: 20) | | | | | | |
| PNS-Tau | WVQEGFLR | 13 | 474.2691 | 2 | 2.55E+08 | 0.00E+00 | 0.00E+00 |
| | (SEQ ID NO: 16) | | | | | | |
| Total-Tau | IGSLDNITHVPGGGNK | 13.8 | 526.946 | 3 | 1.05E+08 | 1.04E+08 | 3.09E+08 |
| | (SEQ ID NO: 187) | | | | | | |

All the features of each aspect and embodiment described above apply mutatis mutandis to all other aspects and embodiments of the present invention.

1. A method for quantifying by mass spectrometry the amount of a subtype of the Microtubule Associate Protein Tau that is present in a biological sample from a subject by measuring the absolute quantity of at least one target peptide that is substantially unique to the subtype being quantified with reference to at least one corresponding reference peptide(s), in a method comprising the steps:
(i) either, adding at least one reference peptide or at least one precursor reference peptide(s) or at least one reference polypeptide to the biological sample followed by incubating the biological sample with at least one protease to form a proteolytic digest of Tau proteins in the sample so that the protease releases the at least one target peptide from the Tau subtypes present and also releases the reference peptide from the precursor reference peptide or reference polypeptide; followed by isolating the Tau peptides and reference peptides from the proteolytic digest of the sample to form a target proteolytic digest;
or, incubating the biological sample with at least one protease to form a proteolytic digest of Tau proteins in the sample so that the protease releases the at least one target peptide from the Tau subtypes present; followed by adding the at least one reference peptide(s) to the proteolytic digest and then isolating the Tau peptides from the proteolytic digest of the sample to form a target proteolytic digest;
(ii) assaying the target proteolytic digest for the presence of the at least one target proteolytic peptide that is substantially unique to the isoform of the Microtubule Associate Protein Tau and the at least one corresponding reference peptide(s), using mass spectrometry; optionally wherein the subtype is the Microtubule Associate Protein Tau expressed by the Central Nervous System or the Microtubule Associate Protein Tau expressed by the Peripheral nervous system.

2. The method according to claim 1 wherein the isoform is the Microtubule Associate Protein Tau expressed by the Central Nervous System and wherein the at least one target proteolytic peptide is one or more polypeptides selected from SEQ ID NO 1 to SEQ ID NO 5 or SEQ ID NO 25 or SEQ ID NO 26 (CNS tau peptides in table 1, 2, 3).

3. The method according to claim 1 wherein the subtype is the Microtubule Associate Protein Tau expressed by the Peripheral Nervous System and wherein the at least one target proteolytic peptide is one or more polypeptides selected from SEQ ID NO: 6 to SEQ ID NO 13 or SEQ ID NO 16 to SEQ ID NO 20 or SEQ ID NO 27 to SEQ ID NO 33. (PNS tau peptides in table 1, 2, 3).

4. The method according to any previous claim wherein the reference peptides comprises one or more peptides each comprising an amino acid sequence independently selected from SEQ ID NO: 43 to SEQ ID NO: 78.

5. The method according to any previous claim wherein the precursor reference peptides comprises one or more peptides each comprising an amino acid sequence independently selected from SEQ ID NO: 79 to SEQ ID NO: 186.

6. The method according to any previous claim wherein the reference polypeptides comprises one or more polypeptides selected from Table 7.

7. The method according to any previous claim wherein the at least one protease is trypsin, optionally in combination with Lys-C and/or AspN.

8. The method according to any previous claim, wherein at least one of the one or more target proteolytic peptides comprises the sequence DEAAGHVTQAR (SEQ ID NO: 25) and/or the sequence VQIVYKPV (SEQ ID NO: 26) and wherein the subtype is the Microtubule Associate Protein Tau expressed by the Central Nervous System.

9. The method according to any previous claim, wherein at least one of the one or more reference peptides, precursor reference peptides or reference polypeptides comprise heavy isotopes.

10. The method according to claim 9, wherein the heavy isotopes are selected from ²H, ¹³C, ¹⁵N or ¹⁸O.

11. A method according to claims 1 to 10 for diagnosing and/or classifying the severity of a neurodegenerative disease in a subject, the method comprising:
(i) Quantifying the absolute amount of the subtypes of the Microtubule Associate Protein Tau that are expressed by the Central Nervous System present in a biological sample from the subject by measuring the absolute quantity of at least one peptide that is substantially unique to the subtypes of the Microtubule Associate Protein Tau that are expressed by the Central Nervous System in a method comprising the steps:
(a) preparing the biological sample from the subject for assay by adding a predetermined amount of reference peptides or polypeptides to the sample;
(b) isolating Tau proteins and the reference peptides or precursor reference peptides or reference polypeptides from the sample; then incubating the isolated target proteins and reference peptides or polypeptides with a protease to form a target proteolytic digest; or
(c) incubating the sample with a protease to form a proteolytic digest of proteins and reference peptides or precursor reference peptides or reference polypeptides in the sample; and then isolating the target peptides and reference peptides from the proteolytic digest of the sample to form a target proteolytic digest;
(ii) assaying the target proteolytic digest of step (i) for the presence of at least one target proteolytic peptide selected from a panel of peptides derived from the subtypes of the Microtubule Associate Protein Tau that are expressed by the Central Nervous System, wherein the presence of said at least one target proteolytic peptide(s) is assayed for using mass spectrometry with reference to the corresponding reference proteolytic peptide or polypeptide; and
(iii) diagnosing and/or classifying the severity of the neurodegenerative disease of the subject according to the relative concentration of at least one target proteolytic peptide measured in the sample.

12. The method according to claim 11, wherein the proteolytic digest is generated by contacting the sample with the protease Trypsin or Trypsin combined with LysC.

13. The method according to claims 11 and 12, wherein the at least one target proteotypic peptide to be assayed from the target proteolytic digest comprises at least one target proteolytic peptide selected from the group of peptides consisting of:
(SEQ ID NO: 1)
AEEAGIGDTPSLEDEAAGHVTQAR
(SEQ ID NO: 2)
QAAAQPHTEIPEGTTAEEAGIGDTPSLEDEAAGHVTQAR
(SEQ ID NO: 3)
STPTAEAEEAGIGDTPSLEDEAAGHVTQAR
(SEQ ID NO: 4)
VQIVYKPVDLSK
(SEQ ID NO: 5)
EAGIGDTPSLEDEAAGHVTQAR

14. The method according to claim 11, wherein the proteolytic digest is generated by contacting the sample with the protease Trypsin combined with AspN.5. The method according to claims 1 and 4, wherein the at least one target proteotypic peptide to be assayed from the target proteolytic digest comprises at least one target proteolytic peptide selected from the group of peptides consisting of:
(SEQ ID NO: 25)
DEAAGHVTQAR
(SEQ ID NO: 26)
VQIVYKPV

15. A method according to claims 1 to 10 for diagnosing and/or classifying the severity of a neurodegenerative disease in a subject, the method comprising:
(i) Quantifying the absolute amount of the subtype of the Microtubule Associate Protein Tau that are expressed by the Central Nervous System present in a biological sample from the subject by measuring the absolute quantity of at least one peptide that is substantially unique to the subtypes of the Microtubule Associate Protein Tau that are expressed by the Peripheral Nervous System and also measuring the absolute quantity of the Total amount of the Microtubule Associate Protein Tau by measuring of at least one peptide that is common to substantially all of the subtypes of Microtubule Associate Protein Tau and then subtracting the quantity of the Tau expressed by the Peripheral Nervous System from the quantity of Total Tau in a method comprising the steps:
(a) preparing the biological sample from the subject for assay by adding a predetermined amount of reference peptides or polypeptides to the sample;
(b) isolating Tau proteins and the reference peptides or polypeptides from the sample; then incubating the isolated target proteins and reference peptides or precursor reference peptides or reference polypeptides with a protease to form a target proteolytic digest; or
(c) incubating the sample with a protease to form a proteolytic digest of proteins and reference peptides or precursor reference peptides or reference polypeptides in the sample; and then isolating the target peptides and reference peptides from the proteolytic digest of the sample to form a target proteolytic digest;
(ii) assaying the target proteolytic digest of step (i) for the presence of at least one target proteolytic peptide selected from a panel of peptides derived from the subtypes of the Microtubule Associate Protein Tau that are expressed by the Peripheral Nervous System and at least one target proteolytic peptide selected from a panel of peptides that are common to substantially all the subtypes of the Microtubule Associate Protein Tau, wherein the presence of said at least one target proteolytic peptide(s) is quantified using mass spectrometry with reference to the corresponding reference proteolytic peptide or polypeptide; and
(iii) diagnosing and/or classifying the severity of the neurodegenerative disease of the subject according to the relative concentration of at least one target proteolytic peptide measured in the sample.

16. The method according to claim 15, wherein the proteolytic digest is generated by contacting the sample with the protease Trypsin or Trypsin combined with LysC.

17. The method according to claims 15 and 16, wherein the at least one target proteotypic peptide derived from the subtypes of the Microtubule Associate Protein Tau that are expressed by the Peripheral Nervous System to be assayed from the target proteolytic digest comprises at least one target proteolytic peptide selected from the group of peptides consisting of:
(SEQ ID NO: 6)
QPSGTGPEDTEGGR
(SEQ ID NO: 7)
DVDESSPQDSPPSK
(SEQ ID NO: 8)
ASPAQDGRPPQTAAR
(SEQ ID NO: 9)
EATSIPGFPAEGAIPLPVDFLSK
(SEQ ID NO: 10)
VSTEIPASEPDGPSVGR
(SEQ ID NO: 11)
GQDAPLEFTFHVEITPNVQK
(SEQ ID NO: 12)
GPSLGEDTK
(SEQ ID NO: 13)
EADLPEPSEK
(SEQ ID NO: 16)
VVQEGFLR
(SEQ ID NO: 17)
HAPELLK
(SEQ ID NO: 18)
HQLLGDLHQEGPPLK
(SEQ ID NO: 19)
EQAHSEEHLGR
(SEQ ID NO: 20)
AAFPGAPGEGPEAR

18. The method according to claims 15 and 16, wherein the at least one target proteotypic peptide that is common to substantially all the subtypes of the Microtubule Associate Protein Tau to be assayed from the target proteolytic digest comprises at least one target proteolytic peptide selected from the group of peptides consisting of:
(SEQ ID NO: 14)
LQTAPVPMPDLK
(SEQ ID NO: 15)
TDHGAEIVYK
(SEQ ID NO: 21)
GAAPPGQK
(SEQ ID NO: 22)
GQANATR
(SEQ ID NO: 23)
HQPGGGK
(SEQ ID NO: 24)
HVPGGGSVQIVYKPVDLSK
(SEQ ID NO: 187)
IGSLDNITHVPGGGNK

19. The method according to claim 15, wherein the proteolytic digest is generated by contacting the sample with the proteases Trypsin combined with AspN or a combination of Trypsin, Lys-C and AspN.

20. The method according to claims 15 and 19, wherein the at least one target proteotypic peptide derived from the subtypes of the Microtubule Associate Protein Tau that are expressed by the Peripheral Nervous System to be assayed from the target proteolytic digest comprises at least one target proteolytic peptide selected from the group of peptides consisting of:
(SEQ ID NO: 16)
VVQEGFLR
(SEQ ID NO: 17)
HAPELLK
(SEQ ID NO: 18)
HQLLGDLHQEGPPLK
(SEQ ID NO: 19)
EQAHSEEHLGR
(SEQ ID NO: 20)
AAFPGAPGEGPEAR
(SEQ ID NO: 27)
DEAAGHVTQEPESGK
(SEQ ID NO: 28)
DTEGGR
(SEQ ID NO: 29)
DLHQEGPPLK
(SEQ ID NO: 30)
DGRPPQTAAR
(SEQ ID NO: 31)
DGPSVGR
(SEQ ID NO: 32)
DAPLEFTFHVEITPNVQK
(SEQ ID NO: 33)
DLPEPSEK

21. The method according to claims 15 and 19, wherein the at least one target proteotypic peptide that is common to substantially all the subtypes of the Microtubule Associate Protein Tau to be assayed from the target proteolytic digest comprises at least one target proteolytic peptide selected from the group of peptides consisting of:
(SEQ ID NO: 21)
GAAPPGQK
(SEQ ID NO: 22)
GQANATR
(SEQ ID NO: 23)
HQPGGGK
(SEQ ID NO: 34)
DNITHVPGGGNK
(SEQ ID NO: 35)
DHGAEIVYK
(SEQ ID NO: 36)
DEVSASLAK
(SEQ ID NO: 188)
HVPGGGSVQIVYKPV

22. The method according to any preceding claim, wherein the step of preparing a biological sample from the subject by isolating target proteins from the sample includes enrichment of the target proteins.

23. The method according to claim 22, wherein the enrichment of the target proteins is carried out by contacting the biological sample from the subject with a solid phase support wherein the support is coated with protein affinity ligands with a binding affinity for the target proteins such that the target proteins bind to the solid phase support allowing extraneous sample material to be washed away.

24. The method according to claim 23 wherein the solid phase support is a magnetic microparticle or nanoparticle.

25. The method according to claim 23 or 24 wherein the protein affinity ligand is selected from an Immunoglobulin G antibody, an aptamer, a peptide, an affibody, a small molecule ligand.

26. The method according any of the preceding claims wherein the reference polypeptides are recombinant versions of the target proteins.

27. The method according any of the preceding claims wherein the reference peptides, precursor reference peptides or polypeptides are synthetic peptides.

28. The method according to claim 26 or 27 wherein the reference peptides or polypeptides or have been modified to incorporate stable heavy isotopes.

29. The method according to claim 28 wherein the stable heavy isotopes are selected from ²H, ¹³C, ¹⁵N or ¹⁸O.

30. The method according any of the preceding claims wherein the biological sample from the subject is Whole Blood, Blood Plasma, Serum or Saliva.

31. A method for diagnosing and/or classifying the severity of a neurodegenerative disease in a subject, the method comprising:
(i) determining the absolute quantity of Central Nervous System Tau according to any one of the previous claims:
(ii) diagnosing and/or classifying the severity of the neurodegenerative disease of the subject according to the absolute quantity of Central Nervous System Tau.

32. A method for the treatment of a subject with a Neurodegenerative disease, the method comprising:
determining the absolute quantity of subtypes of Microtubule Associated Protein Tau expressed by the Central Nervous System according to claims 1 to 30 in a biological sample from the subject;
diagnosing and/or classifying the severity of the neurodegenerative disease of the subject according to the absolute quantity of Central Nervous System Tau measured in the sample; and
treating the subject in need thereof with a therapeutic agent or treatment according to the severity of the Neurodegenerative disease detected in the subject.

33. A method for assessing the efficacy of a treatment for a neurodegenerative disease in a subject, the method comprising:
determining the absolute quantity of subtypes of Microtubule Associated Protein Tau Expressed by the Central Nervous System according to claims 1 to 30 in a biological sample from the subject before treating the subject with a therapeutic agent;
treating the subject in need thereof with a therapeutic agent
determining at one or more further timepoints, after treatment of the subject with a therapeutic agents, the absolute quantity of subtypes of Microtubule Associated Protein Tau Expressed by the Central Nervous System according to claims 1 to 21 in a biological sample from the subject before treating the subject with a therapeutic agent;
assessing the effectiveness of the treatment based on whether the levels of Microtubule Associated Protein Tau Expressed by the Central Nervous System in the biological samples are reduced.

34. The method according any of the preceding claims, wherein the Neurodegenerative disease is Alzheimer's disease.

35. A kit for diagnosing and/or classifying the severity of a Neurodegenerative disease in a subject, comprising:
a plurality of sample preparation media for analysis of a biological sample from a subject by mass spectrometry for the presence of at least one target proteolytic peptide selected from the group of peptides from subtypes of Microtubule Associated Protein Tau Expressed by the Central Nervous System, subtypes of Microtubule Associated Protein Tau Expressed by the Peripheral Nervous System and peptides that are common to all subtypes of Microtubule Associated Protein Tau.

36. A kit for use in the treatment of a subject with a Neurodegenerative disease, wherein the kit comprises:
a plurality of sample preparation media for analysis of a biological sample from a subject by mass spectrometry for the presence of at least one target proteolytic peptide selected from the group of peptides from subtypes of Microtubule Associated Protein Tau Expressed by the Central Nervous System, subtypes of Microtubule Associated Protein Tau Expressed by the Peripheral Nervous System and peptides that are common to all subtypes of Microtubule Associated Protein Tau.; and
a therapeutic agent for treatment of a Neurodegenerative disease.

37. A kit for assessing the efficacy of a treatment for a neurodegenerative disease in a subject, wherein the kit comprises:
a plurality of sample preparation media for analysis of a biological sample from a subject by mass spectrometry for the presence of at least one target proteolytic peptide selected from the group of peptides from Subtypes of Microtubule Associated Protein Tau Expressed by the Central Nervous System, Subtypes of Microtubule Associated Protein Tau Expressed by the Peripheral Nervous System and peptides that are common to all Subtypes of Microtubule Associated Protein Tau.

38. The kit according to any of claims 35 to 37, further comprising one or more proteases selected from the group consisting of Trypsin or a combination of Trypsin and Endoproteinase Lys-C, Trypsin and Asp-N or a combination of all three of Trypsin and Endoproteinase Lys-C and AspN.

39. The kit according to any of claims 35 to 38, wherein the kit comprises reference peptides or polypeptides.

40. The kit according to claim 39, wherein the reference peptides or polypeptides are recombinant versions of the target proteins.

41. The kit according to claim 40, wherein the reference peptides or polypeptides are synthetic peptides.

42. The kit according to claim 39 to 41, wherein the reference peptides or polypeptides comprise stable heavy isotopes selected from ¹³C, ¹⁵N, ²H or ¹⁸O.

43. The kit according to claims 35 to 42, wherein the kit comprises solid phase supports derivatised with Protein Affinity Ligands, where there is at least one solid phase support with one Protein Affinity Ligand that binds to the Microtubule-Associated Protein Tau (MAPT).

44. The kit according to claim 43, wherein the kit comprises 2 or more solid phase supports each derivatized with a different Protein Affinity Ligand binding 2 or more of the target proteins are included in the kit.

45. The kit according to claims 35 to 44, wherein the kit contains machine-readable media containing mass spectrometric transitions or target mass spectra for the target peptides.
